(19) United States
(12) Patent Application Publication
(10) Pub. No.: US 2014/0234903 A1
(43) Pub. Date: Aug. 21, 2014

(54) BIOSYNTHETIC GENE CLUSTER FOR THE PRODUCTION OF PEPTIDE/PROTEIN ANALOGUES

(71) Applicants: Jorn Piel, Zurich (CH); Cristian Gurgui, Rottendorf (DE); Michael Francis Freeman, Zurich (CH); Agustinus Robert Uria, Zurich (CH); Maximilian Johannes Helf, Zurich (CH)

(72) Inventors: Jorn Piel, Zurich (CH); Cristian Gurgui, Rottendorf (DE); Michael Francis Freeman, Zurich (CH); Agustinus Robert Uria, Zurich (CH); Maximilian Johannes Helf, Zurich (CH)

(73) Assignee: ETH ZURICH, Zurich (CH)

(21) Appl. No.: 14/342,539

(22) PCT Filed: Sep. 5, 2012
(86) PCT No.: PCT/EP2012/067280

§ 371 Date: Mar. 4, 2014

(30) Foreign Application Priority Data
Sep. 5, 2011 (EP) 11180107.2
Aug. 15, 2012 (EP) 12180565.9

(51) Int. Cl. C12N 15/52; C12N 9/02; A61K 47/48; C07K 14/435; C07K 16/28; C12N 9/10; C12N 9/88

(52) U.S. Cl. CPC C12N 15/52; C12N 9/1007; C12N 9/0069; C12N 9/88; C07K 14/43504; C07K 16/28; A61K 47/48569
USPC 435/69.1; 536/23.2; 435/320.1; 536/24.33; 435/252.3; 435/193; 435/189; 435/232; 530/324; 530/387.9; 530/391.7

(57) ABSTRACT

A novel gene cluster encoding polypeptides involved in the generation of bio synthetically unique peptide-based compounds is described. In addition, new methods for biosynthetic engineering and production of modified peptides and proteins are disclosed. Furthermore, new tools for identification of homologue polypeptides and precursor peptides in other species are provided. In addition, peptide-based compounds and fusions of these compounds with functional moieties for treatment of disorders such as tumors are disclosed.

### FIELD OF THE INVENTION

The present invention generally relates to the provision of a gene cluster encoding novel polypeptides involved in the generation of biosynthetically unique peptide-based compounds, particularly polytheonamides, the precursor peptide thereof and to new methods enabling provision of the products encoded by these sequences. In addition, it is a particular object of the present invention to provide new methods for biosynthetic engineering and production of modified peptides and proteins. Furthermore, new tools for identification of homologue polypeptides and precursor peptides are provided. In addition the present invention relates to the provision of polytheonamides, other peptide-based compounds and fusions of these compounds with functional moieties for treatment of disorders such as tumors.

### BACKGROUND OF THE INVENTION

Invertebrates, particularly those from marine environments, are an important source of natural products with high therapeutic potential. Examples of such metabolites are polyketides providing a wide range of different drug classes, such as antibiotics (erythromycin A), immunosuppressants (rapamycin), antifungal (amphotericin B), antiparasitic (avermectin) and anticancer (doxorubicin) drugs Vaishnav and Demain, Biotechnol Adv. 29 (2011), 223-229; terpenes for use as anticancer and/or virostatic drugs (havarol, havarone) Cimino et al., Experientia 38 (1982), 896; Cozzolino et al., J. Nat. Prod. 1990, 53, 699-702; Müller et al., Cancer Res. 1985, 45, 4822-4826; Müller et al., Eur. J. Cancer Clin. Oncol. 1986, 22, 473-476; Müller et al., Biochem. Pharmacol. 1987, 36, 1489-1494; Sarin et al., J. Natl. Cancer Inst. 1987, 78, 663-666; alkaloids as anticancer drugs (ecteinascidin 743) or as antibiotics (8-hydroxymanzamine) Rao et al., J Nat Prod. 66 (2003), 823-828; Rinehart et al., J. Org. Chem. 55 (1990), 4512-4515; Peng et al., J Med Chem. 53 (2010), 61-76; Sakai et al., Proc. Natl. Acad. Sci. U.S.A. 89 (1992), 11456-11460; and polytheonamides which by their cytostatic capabilities show as well high anticancer potential (Hamada et al., J. Am. Chem. Soc. 127 (2005), 110-118; Hamada et al., J. Am. Chem. Soc. 132 (2010), 12941-12945; Iwamoto et al., J. Physiol. Sci. 60 (2010), S121; Iwamoto et al., FEBS Lett. 584 (2010), 3995-3999; Inoue et al., Nat. Chem. 2 (2010), 280-285).

The low availability of most of these metabolites, however, represents a serious impediment to drug development. As many invertebrates are difficult to cultivate and chemical synthesis is usually not economical, alternative and ecologically friendly sources of natural products are urgently needed. The actual producers of many drug candidates isolated from the invertebrates may well be symbiotic bacteria, but so far no producing symbiont has ever been successfully cultured.

Therefore, the technical problem underlying the present invention was to provide means and methods for the reliable and easy production of natural products and compounds derived thereof with high therapeutical potential, such as cytotoxic compounds which may be used, e.g., in tumor treatment.

This technical problem has been solved by the embodiments as characterized in the claims and described further below.

### SUMMARY OF THE INVENTION

Marine sponges belong to the richest known sources of bio active natural products (Faulkner, Nat. Prod. Rep. 17 (2000), 1-6; Blunt et al., Nat. Prod. Rep. 27 (2010), 165-237). Many of these compounds are highly cytotoxic complex polyketides or modified peptides. Among the numerous natural products which have been found in specimens of the marine sponge Theonella swinhoei particularly interesting are structurally intriguingly modified peptides of the polytheonamide series (FIG. 1), which belong to the largest known class of secondary metabolites (Hamada et al., J. Am. Chem. Soc. 127 (2005), 110-118). Of 48 amino acid residues, 26 are non-proteinogenic. Common modifications are methylations (at least 14 of these at non-activated positions), hydroxylations and epimerizations. In total, 18 residues exhibit a D-configuration and are located at positions that perfectly alternate with L-configured residues. As a consequence of these modifications, the peptide adopts a hydrophobic β-helix that inserts into membranes and forms a minimalistic ion channel, resulting in an extremely high cytotoxicity in the low picomolar range (Hamada et al., J. Am. Chem. Soc. 132 (2010), 12941-12945; Iwamoto et al., J. Physiol. Sci. 60 (2010), S121; Iwamoto et al., FEBS Lett. 584 (2010), 3995-3999; Inoue et al., Nat. Chem. 2 (2010), 280-285).

Regardless of the high potential of polytheonamides due to their cytotoxic properties, approaches for use of these compounds have failed so far due to problems with their isolation and/or low production rate from the marine sponge Theonella swinhoei. Furthermore, the biosynthesis of polytheonamides has not been clarified yet. Since extensive C-methylations and epimerizations are unknown from ribosomal peptides, it is generally believed that polytheonamides are the products of a non-ribosomal peptide synthetase of enormous size (Hamada et al., J. Am. Chem. Soc. 127 (2005), 110-118; Hamada et al., J. Am. Chem. Soc. 132 (2010), 12941-12945; Iwamoto et al., J. Physiol. Sci. 60 (2010), S121; Iwamoto et al., FEBS Lett. 584 (2010), 3995-3999; Inoue et al., Nat. Chem. 2 (2010), 280-285).

However, during the search for new natural products of pharmacological value, an analysis of the sponge metagenomic DNA (=consisting of the sponge genomic DNA and many genomes of the multispecies community living on and in the sponges) surprisingly revealed a gene for a predicted peptide sequence that precisely matches the structure of a hypothetical unmodified polytheonamide precursor.

For some novel natural products, e.g., various polyketides, Piel and others (Piel et al., Proc. Natl. Acad. Sci. U.S.A. 101 (2004), 16222-16227; Fisch et al., Nat. Chem. Biol. 5 (2009), 494-501; Nguyen et al., Nat. Biotechnol. 26 (2008), 225-233; Taylor et al., Microbiol. Mol. Biol. Rev. 71 (2007), 295-347) have recently shown that the true producers are as-yet unculturable symbiotic bacteria, which are part of massive, multispecies communities present in the animal tissues. Subsequently to the discovery of the polytheonamide precursor peptide, a small biosynthetic gene cluster (poy-cluster; SEQ ID NO: 1) was isolated from the total sponge DNA that was attributed to a bacterium and contains, in addition to the peptide precursor gene, genes encoding an S-adenosylmethionine-(SAM-)dependent methyltransferase, an oxygenase, three proteins of the radical SAM superfamily (Frey et al., Crit Rev Biochem Mol Biol. 43 (2008), 63-88; Sofia et al., Nucl. Acids Res. 29 (2001), 1097-1106), a LanC-type lantibiotic dehydratase/cyclase (Bierbaum and Sahl, Curr. Pharm. Biotechnol. 10 (2009), 2-18) and a transporter (see Tables 1 and 2 for the sequences of the cluster, the respective genes and the particular biological functions of the particular gene products of the gene cluster; in this respect see also the description of FIG. 2).

Modified ribosomal peptides, albeit with different modifications, are also known from other bacterial pathways (Oman and van der Donk, Nat. Chem. Biol. 6 (2010), 9-18; McIntosh, et al., Nat. Prod. Rep. 26 (2009), 537-559). In all cases, the precursor peptide contains an external N-terminal region, termed leader peptide that is recognized by the tailoring enzymes. After modification, the leader peptide is cleaved off during or after export out of the cell, thus releasing the final natural product. Leader peptide sequences have been used to classify modified ribosomal peptides into families (Oman and van der Donk, Nat. Chem. Biol. 6 (2010)) such as lantibiotics (Bierbaum and Sahl, Curr. Pharm. Biotechnol. 10 (2009), 2-18), microcins (Severinov et al., Mol. Microbiol. 65 (2007), 1380-1394), thiopeptides (Arndt et al., Angew. Chem. Int. Ed. 48 (2009), 6770-6773) and cyanobactins (Donia et al., Nat. Chem. Biol. 4 (2008), 341-343). Usually, characteristic modifications are found in individual families, such as lanthionine residues in lantibiotics, pyridine moieties in thiopeptides and macrocycles and prenylation in cyanobactins.

In the case of the polytheonamides, which bear no resemblance to peptides from known families, the precursor peptide contains a unique leader region that exhibits similarity to nitrile hydratases. Interestingly, Haft et al. (BMC Biol. 8:70 (2010)) recently discovered genes encoding similar leader peptides in the sequenced genomes of ten other bacteria. Based on these in silico studies they proposed the existence of a new family of natural products (NHLP, nitrile hydratase leader peptide family). Since no metabolite has been attributed to the identified gene clusters, polytheonamides are the as-yet only characterized NHLP members. With respect to the highly modified structure of these compounds and the marine origin of polytheonamides the inventors of the present invention propose the name proteusins for this new family (from Proteus, a Greek sea-god with the ability to shape-shift beyond recognition).

Thus in general, the present invention relates to methods for biosynthetic engineering and production of modified peptides and proteins. In particular, novel genes encoding polypeptides which catalyze at least one step of the biosynthesis of polytheonamides, and/or the precursor peptide thereof and the corresponding gene products are provided. Moreover, the present invention relates to vectors, host cells, antibodies, and recombinant methods for producing the novel polypeptides which catalyze at least one step of the biosynthesis of polytheonamides and/or the precursor peptide thereof or other peptides which are to be subjected to the enzymatic activities of the polypeptides of the isolated Poy-polypeptides encoding cluster.

Furthermore, the present invention makes general use of the finding that polytheonamides as extensively modified peptides with 48 residues of which 22 are nonproteinogenic, are synthesized via a remarkable pathway that involves numerous posttranslational modifications such as epimerizations, hydroxylations and methylations of a ribosomal precursor peptide. No apparent correlation exists between the type of modification and the structure of the modified unit. For example, hydroxylations are performed on three different residues of the precursor peptide, and at least five types of residues are methylated. Conversely, identical amino acids are modified at some positions, while they remain unchanged at others (e.g., Thr and Gln appear in three different variants each). This relaxed substrate specificity reconciled with precise biosynthetic control is one of the most fascinating aspects of the polytheonamide pathway and has, by the usage of the respective polypeptides of the present invention important consequences for the rational generation of peptides and proteins with unprecedented modifications. The apparent capability to convert multiple residues at different positions highlights the benefit of the polypeptides encoded by the genes of the poy-cluster in the development of sustainable biotechnological production systems for drug candidates that are currently only available at very limited amounts.

Provided by the present invention, the genes of the poy-cluster (SEQ ID No:1) may be produced by heterologous expression in culturable bacteria instead of the as-yet non-culturable bacterial endobionts of T. swinhoei in industrial amounts and purity required by the rules of GMP, e.g., also by the addition of fusion-tags allowing their purification by means such as affinity-purification methods. Furthermore, by the use of peptide precursors modified in respect to the original peptide sequence of PoyA (SEQ ID NO: 3) engineering of new modified peptides and proteins is possible. This is envisaged, according to the methods of the present invention by in-vitro or in-vivo treatment of modified and/or new peptide substrates with the enzymes encoded by the genes of the poy-cluster.

Also provided are methods for treating disorders such as tumors. The present invention further relates to screening methods for identifying homologous polypeptides which are capable of catalyzing at least one step in the biosynthesis of polytheonamides and for identifying new precursor peptides thereof.

### BRIEF DESCRIPTION OF THE DRAWINGS

FIG. 1A: Ribosomal biosynthesis of polytheonamides as postulated.
Hypothetical transcripts of the underlined regions at the ends of the postulated precursor peptide were used as degenerate oligonucleotides in PCR-based cloning of the cDNA encoding the precursor peptide from metagenomic DNA isolated from T. swinhoei and its endobionts (Degenerated oligonucleotides F1=Polytheo-For1: for peptide sequence GIGVVVA—SEQ ID NO: 68; F2=Polytheo-For2: for peptide sequence GAGVNQT—SEQ ID NO: 69; R1=Polytheo-Rev: (for peptide sequence VNMNQTT—SEQ ID NO: 70—see for oligonucleotide sequences in Table 2.
A glycine-repeat, preceding a potential cleavage site is marked in bold. Below, the biosynthesis of polytheonamides as postulated.

FIG. 1B: Chemical structure of polytheonamides. Polytheonamids A and B are epimers differing in the configuration of the sulfoxide of the 44^{th} residue. Further modifications are indicated as follows: e=epimerizations, Me=methylations, OH-hydroxylations, dh=Dehydroxylation; D marks the D-configured amino acid residues.

FIG. 2: The polytheonamide biosynthesis gene cluster (poy-cluster). Grey shadowed genes encode for tranposases which are involved in the biosynthesis of polytheonamides. Products of the poy genes show homologies to genes with known biological functions as indicated. poyK—Unknown; poyJ—Putative transporter/hydrolase; poyB and poyC—Putative radical SAM methyltransferases; poyA—Precursor peptide; poyD—Radical SAM-dependent enzyme; poyE—SAM-dependent methyltransferase (nucleophilic); poyF—LanM, N-terminal dehydratase domain; poyG—Chagasin-like peptidase inhibitor; poyH—C1 peptidase; poyI—Fe(II)/alpha-ketoglutarate-dependent oxygenase

FIG. 3: Mass spectrometric identification of poyF-catalyzed dehydration. To identify the modified residue. LC/MS/MS analysis of peptides resulting from trypsinisation of PoyA purified from coexpression with PoyDF was performed. The C-terminal tryptic peptide corresponding to residues 76-145 of PoyA was found to contain the dehydration site at residue Thr-97. (A) Deconvoluted ESI-MS spectrum of PoyA exhibiting a mass shift of −18 Da. (B) ESI-MS/MS spectrum of tryptic peptide 76-145 from PoyA (precursor ion [M+6H]⁶⁺ at m/z 1130.560; calculated m/z 1130.562 (monoisotopic)) showing a series of b-type fragment ions.

FIG. 4: Model for the formation of the N-acyl terminus from threonine. The final cleavage is probably performed by PoyH/J.

FIG. 5: Amplification of a portion of poyA encoding the polytheonamide precursor peptide from the sponge metagenomic DNA by semi-nested PCR. Peptide sequences corresponding to the expected amplicons are shown above each gel. Regions used for the design of respective primer pairs (Polytheo-For1, Polytheo- and Polytheo-Rev as indicated in the description of FIG. 1, above) are highlighted. The arrow indicates the size of the expected amplicon. (A) Amplicons generated in the first round. The strong bands are unspecific PCR products. (B) Second round of PCR using the excised 144 bp region of (A) as template. Lanes in both gels: Lane 1: PCR at 42° C.; Lane 2: 42.4° C.; Lane 3: 43.5° C.; Lane 4: 54.2° C.; Lane 5: 47.1° C.; Lane 6: 49° C.; M: 1 kb DNA size marker. The negative control did not contain template DNA.

FIG. 6: 15% SDS-PAGE of Nhis-PoyA purification from co-expression with poyD in BL21(DE3)star pLysS. Lane M: molecular weight ladder (kD); Lane 1: uninduced cellular fraction; Lane 2: induced cellular fraction; Lane 3: lysis pellet fraction; Lane 4: lysis supernatant fraction; Lane 5: 250 mM imidazole elution fraction. Nhis-PoyA (17.5 kD) is indicated with an arrow.

FIG. 7: HPLC chromatograms for detection (λ=340 nm) of L-FDVA-derivatized (A) aspartic acid and (B) valine from acid hydrolysates of Nhis-tagged proteins. 1st chromatogram, Nhis-poyA+poyD coexpression; 2nd chromatogram, Nhis-poyA121 (residues 1-121 containing the first 24 core amino acids)+poyD coexpression; 3rd chromatogram, Nhis-poyA101 (residues 1-101 containing only the first five core amino acids)+poyD coexpression; 4th chromatogram, Nhis-poyA101; 5^{th} chromatogram (black), derivatized standards. All peaks were normalized to (A) L-Asp and (B) L-Val. D-Amino acid percentages (adjacent to chromatograms) were derived from peak areas [D/(D+L)]*100. The D-Asp and D-Val detected in the 3rd and 4th chromatograms is attributed to racemization that occurs during acid hydrolysis (Kaiser and Benner, Limnol. Oceanogr. Meth. 3 (2005), 318-325). The lack of additional epimerization (aside from previously reported background racemization) in the 3rd and 4th chromatograms pinpoint PoyD as the enzyme responsible for multiple and different amino acid epimerizations and that only amino acids within the core region of PoyA are modified. Taking into account background racemization, roughly 8 of 10 Asn and 1 of 5 Val in Nhis-PoyA were epimerized, while approximately 2 of 2 Asn and 3 of 4 Val were epimerized in Nhis-PoyA121. The additional epimerizations seen in Nhis-PoyA121 are presumably due to additional posttranslational modifications needed prior to full epimerization of Nhis-PoyA. The incomplete epimerization of 8 Asn and a single Val observed with the full-length Nhis-PoyA construct is consistent in amino acid composition with the C-terminal half of polytheonamides epimerized. Furthermore, when the C-terminal half was removed in the Nhis-PoyA121 construct, the remaining N-terminal portion was almost fully epimerized. As a result, the amount, composition, and differences in epimerization seen in the Nhis-poyA and Nhis-poyA121 coexpressions are in agreement with near-complete epimerization of all expected asparagines and valines.

FIG. 8: ESI-MS of HPLC-purified, L-FDVA-derivatized Asp and Val from coexpression of Nhis-poyA and poyD. (A) L-Asp, (B) D-Asp, (C) L-Val, and (D) D-Val.

FIG. 9: ESI-mass spectrum (9A: deconvoluted; 9B: raw spectrum) of PoyA from the PoyADE triple expression strain (measured mass: 17456 Da; expected mass 17456 Da).

FIG. 10: ESI-mass spectrum (10A: deconvoluted; 10B raw spectrum) of PoyA from the PoyADF triple expression strain (measured mass: 17090 Da; expected mass for unmodified protein 17108 Da).

FIG. 11: ESI-ECD-MS/MS spectrum of the C-terminal tryptic peptide of PoyA from the PoyADE triple expression strain [M+6H]⁶⁺=m/z 1134, sequencing the peptide and showing the absence of modification.

FIG. 12: ESI-ECD-MS/MS spectrum of the C-terminal tryptic peptide of PoyA from the PoyADF triple expression strain [M+6H]⁶⁺=m/z 1131, sequencing the peptide and showing dehydration.

FIG. 13: (A) LC-ESI/MS spectrum of the C-terminal tryptic peptide 91-160 from Nhis-PoyA following coexpression with the putative N-methyltransferase gene poyE showing the presence of multiple methylations. (B) ECD-MS/MS spectrum of m/z 1138 ([M+6H]⁶⁺), and (C) (QTOF) CID-MS/MS spectrum of m/z 1364 ([M+5H]⁵⁺) locating the methylation sites to Asn136 and between residues 116 and 135 of the peptide, consistent with the methylation pattern of polytheonamides. (D) Results of MS/MS fragmentation with the expected polytheonamides N-methylated asparagines highlighted by arrows. Observed methylated residues are labeled with ‘Me’. Although masses corresponding to zero through 8 methylations were observed in full-length Nhis-PoyA and the tryptic peptide 76-145, the low abundance of methylation at Asn140 prevented observation during MS/MS fragmentation (c37 and c40). In order to verify methylation at Asn140, coexpression of Nhis-poyA121 with poyE, poyD, and poyF was carried out in a 3-day incubation at 16° C. (FIG. 14).

FIG. 14: (A) LC-ESI/ECD-MS/MS spectrum of m/z 1097 ([M+4H]⁴⁺) from the C-terminal tryptic peptide 76-121 of Nhis-PoyA121 (a truncated variant of PoyA missing 24 amino acids from the C-terminus) following coexpression with the N-methyltransferase. (B) Results of MS/MS fragmentation with the expected polytheonamides N-methylated asparagines highlighted by arrows. Observed methylated residues are labeled with ‘Me’. Near quantitative monomethylation was observed in this sample, with no evidence of multiple methylation. ECD fragmentation located the site of methylation exclusively to Asn112 (in full-length PoyA numbering). This position is methylated in polytheomamides, as is Asn21 (Asn118 in full-length PoyA). However, the latter was unmodified in this truncated construct. This is a significant observation, as it strongly suggests that the N-methyltranferase does not modify Asn residues in close proximity to the C-terminus. Indeed, in full-length PoyA and in polytheonamides themselves, the two Asn residues most adjacent to this terminus are not methylated. Thus, the regiospecificity of this enzyme, and therefore the N-methylation pattern in polytheonamides, appears to originate from a critical distance relative to the C-terminus of PoyA.

FIG. 15: (A) Influence of polytheonamide B on the membrane potential of Micrococcus luteus ATCC 4698. The potential was calculated from the distribution of the lipophilic cation tetraphenylphosphonium (TPP⁺) inside and outside the cells. The arrow indicates the time of addition of 10-fold MIC polytheonamide B (squares) and 1 μM nisin (black line). (B) Potassium release from Arthrobacter crystallopoietes DSM 20117 whole cells induced by polytheonamide B added at a concentration of 10-fold MIC (filled squares) and 1-fold MIC (triangle). In the control experiment buffer (open squares) and 1 μM nisin was added (black line).

FIG. 16: Sequence variants of Nhis-PoyA used in this study. The two bottom rows show the sequence corresponding to polytheonamides. The C-termini of Nhis-PoyA101 and Nhis-PoyA121 are labeled as ‘101’ and ‘121’, respectively.

FIG. 17: (A) Polytheonamides A and B differ in the configuration of the sulfoxide moiety in residue 44. The sulfoxide arises from spontaneous oxidation during polytheonamide isolation. Residues are numbered based on the typical notation for polytheonamides (2). The core peptide sequence is indicated by bold letters, with the color red denoting posttranslational epimerization. All other biosynthetic transformations during maturation of the core peptide are colored as: orange, C-methylation; purple, N-methylation; blue, hydroxylation; green, dehydration (FIG. 1C). (B) Map of the polytheonamide (poy) biosynthetic gene cluster.

### DETAILED DESCRIPTION OF THE INVENTION

### Definitions

It is to be noted that the term “a” or “an” entity refers to one or more of that entity; for example, “a polypeptide,” is understood to represent one or more polypeptides. As such, the terms “a” (or “an”), “one or more,” and “at least one” can be used interchangeably herein.

### I. Polypeptides

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, dipeptides, tripeptides, oligopeptides, “peptide,” “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation and derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are homologues, fragments, derivatives, analogs or variants of the foregoing polypeptides and any combinations thereof. The terms “homologue”, “fragment,” “variant,” “derivative” and “analog” when referring to polypeptides of the present invention include any polypeptides which retain at least some catalytic properties of the corresponding native polypeptide or protein. Fragments of polypeptides of the present invention include proteolytic fragments, as well as deletion fragments, in addition to specifically modified fragments discussed elsewhere herein. Variants of polypeptides, peptides and peptide precursors of peptide analogues generated by the methods of the present invention include fragments as described above, and also polypeptides with altered amino acid sequences due to amino acid substitutions, deletions, or insertions. Variants may occur naturally or be non-naturally occurring. Naturally occurring variants may exist as products of allelic genes, i.e. differing genomic nucleic acid sequences of the same gene due to, e.g., missense point mutations. Further, naturally occurring variants may exist in the same or in different species, as products of different genes which variants are encompassed by the term “homologues” due to their highly conserved sequence and conserved biological function. Said non-naturally occurring variants may be produced using art-known mutagenesis techniques. Variant polypeptides may comprise conservative or non-conservative amino acid substitutions, deletions or additions. Derivatives of polypeptides of the present invention, are polypeptides which have been altered so as to exhibit additional features not found on the native polypeptide. Examples include fusion proteins. Variant polypeptides may also be referred to herein as “polypeptide analogs”. As used herein a “derivative” of a polypeptide of the present invention or fragment thereof refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The terms “peptide based compound”, “peptide-like compound” and “peptide analogue” are used interchangeably herein and are intended to refer to the products obtained by the treatment of precursors of these with at least one polypeptide which catalyzes at least one step of the biosynthesis of polytheonamides disclosed herein. Thereby, this term refers both to polytheonamides, as generated by the whole process of their biosynthesis from the “precursor peptide” of polytheonamides (SEQ ID NO 47) and to products obtainable by the treatment of other peptides with one or more of the polypeptides catalyzing at least one step of the biosynthesis of polytheonamides. These other peptides are referred to as “precursor peptides” as well, without any reference to their sequence identity in concern of the precursor peptide of polytheonamides, but as a reference to their transitional status only, before, due to the treatment with said at least one polypeptide which catalyzes at least one step of the biosynthesis of polytheonamides, their amino acids are modified.

### II. Polynucleotides

The term “polynucleotide” is used interchangeably with the term “nucleic acid molecule”, the use of either of them is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a binding molecule, an antibody, or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operable associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operable associated” or “operable linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operable associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operable associated with the polynucleotide to direct cell-specific transcription. Suitable promoters and other transcription control regions are disclosed herein.

### III. Control of Gene Expression

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In this respect, in other preferred embodiments the polypeptides, and/or precursor peptides as well as fragments, variants, or derivatives thereof of the invention may be expressed in eukaryotic cells using polycistronic constructs such as those disclosed in US patent application publication No. 2003-0157641 A1 and incorporated herein in its entirety. In these expression systems, multiple gene products of interest such as different polypeptides, or a peptide precursor end several polypeptides may be produced from a single polycistronic construct. These systems advantageously use an internal ribosome entry site (IRES) to provide relatively high levels of antibodies. Compatible IRES sequences are disclosed in U.S. Pat. No. 6,193,980 which is also incorporated herein. Those skilled in the art will appreciate that such expression systems may be used to effectively produce the precursor peptide and the polypeptides encoded by the genes of the poy-cluster disclosed in the instant application.

However, in another preferred embodiment the polypeptides, and/or precursor peptides as well as fragments, variants, or derivatives thereof of the invention may be expressed in prokaryotic cells. Polycistronic expression is a particular issue of prokaryotes. However, when several genes are to be expressed from one particular promoter in prokaryotes, it is envisaged by the methods of the present invention to provide each of the polycistronic genes by its own Shine-Dalgarno sequence and start codon.

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full-length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operable associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. When expressed in prokaryotic hosts other signal sequences have to be used giving the choice of secretion into the periplasmic space or cytoplasmic expression of a polypeptide of interest. For example, the leader sequence from the PhoA-protein may increase the solubility of the fusion polypeptide and direct it to the periplasmic space in the bacterial host (Huang et al., Journal of Biol Chem 276 (2001), 3920-3928). Other signal peptides which may be used for secretion of heterologously expressed proteins are: Lpp, LamB, LTB, MalE, OmpA, OmpC, OmpF, OmpT, PeIB, PhoE, SpA and Tat signal peptides (Choi and Lee, Appl. Microbiol. Biotechnol. 64 (2004), 625-635, Mergulhao et al., Biotechol. Adv. 23 (2005), 177-202)

As used herein, the terms “linked”, “fused” or “fusion” are used interchangeably. These terms refer to the joining together of two or more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature). Although the reading frame is thus made continuous throughout the fused segments, the segments may be physically or spatially separated by, for example, in-frame linker sequence. For example, polynucleotides encoding a polypeptide of the present invention (polypeptide of interest) may be fused, in-frame, with a polynucleotide encoding another peptide or polypeptide sequence which may be used as a tag (fusion tag) for enhanced solubility and/or for purification of the produced polypeptide fusions. The additional polynucleotide may be positioned in front, behind or even internal in respect of the polynucleotide encoding a polypeptide of the present invention as long as the “fused” polypeptides are co-translated as part of a continuous polypeptide from a transcript of the resulting polynucleotide fusion. The positioning will depend on the kind of tag encoded by the additional polynucleotide, as it is known in the art that some of the tags may be positioned in specific orientation only (N-, C-terminal or internal) as another positions may inhibit a correct folding or function of the polypeptide of interest fused to said tag or the tag may be separated from the polypeptide of interest when fused in a specific position only.

### IV. Tags and Purification

Commonly used solubility tags are for example MBP (MBP=maltose-binding protein (Guan et al., Gene 67 (1988), 21-30), NusA (de Marco et al., Biochem Biophys Res Commun. 322 (2004), 766-771), GST (GST=glutathione S-transferase; Smith and Johnson, Gene 67 (1988), 31-40), thioredoxin (TRX), small ubiquitin-like modifier (SUMO; Butt et al., Protein Expr Purif. 43 (2005), 1-9), ubiquitin (Ub), Skip and T7 protein kinase (Chatterjee and Esposito, Protein Expr. Purif. 46 (2006), 122-129). GST and MBP may be used for both purification and increase of solubility of the generated polypeptide fusions by the MBP's affinity to cross-linked amylose (starch).

For the purification of the polypeptides or precursor-peptides of the present invention several affinity tags as known in the art may be used, such as the above mentioned GST and MBP tags, His-tag (a stretch of several, mostly six to eight Histidine-residues), S-tag (a 15 residue peptide, of the sequence KETAAAKFERQHMDS (SEQ ID NO: 60), derived from the pancreatic ribonuclease), Strep II-tag (streptavidin-recognizing octapeptide), T7-tag, FLAG-tag, HA-tag, c-Myc-tag, DHFR-tag, chitin binding domain, calmodulin binding domain, cellulose binding domain, mystic-tag, PD1 fusion, BCCP fusion, isopeptag, SBP-tag, etc.

Since tags which are increasing the solubility of a polypeptide or are used for its purification may affect its biological function or may need to be removed due to, e.g., GMP requirements in pharmacological applications, endopeptidase/protease recognition sequences may be introduced in between the polynucleotide/gene of interest and the fusion partner according to the methods of the present invention allowing the separation of the polypeptide of interest from the fused tags. Specific endopeptidases/proteases recognizing said sequence may be used then for the separation of the polypeptide of interest, i.e. a polypeptide or precursor peptide of the present invention as described hereinabove and the fused tags by a cleavage of the peptide bond between them. Examples for such endopeptidases/proteases include the TEV (tobacco etch virus) protease; thrombin (factor IIa, fIIa) and factor Xa (fXa) from the blood coagulation cascade (Jenny et al., Protein Expr Purif 31 (2003), 1-11); enterokinase (EK; an enzyme involved in the cleavage or activation of trypsin in the mammalian intestinal tract); proteases involved in the maturation and deconjugation of SUMO, SUMO proteases (Ulp1, Senp2, and SUMOstar); and a mutated form of the Bacillus subtilis protease, subtilisin BPN′ (Bio-Rad's Profinity eXact system; Ruan et al., Biochem 43 (2004), 14539-14546) and modified versions thereof with enhanced specificity and/or stability. However, due to the use of endopeptidases unspecific cleavage may occur at cryptic sites or during long treatment endangering the intactness of the tagged peptide, polypeptide or protein. In this respect exopeptidases may be used which remove the tag only. In this respect the TAGZyme™ (QIAGEN®; Hilden, Germany) enzymatic system may be used, comprising the engineered dipeptidyl peptidase I (DAPase™) recombinant exopeptidase. TAGZyme™ cleaves sequentially dipeptides from the N-terminus, provided the amino acid sequence does not contain an arginine or lysine at N-terminus or at an uneven position in the sequence or a proline anywhere in the tag. In case any of these amino acids is present, the enzyme will stall cleavage at this position (see also Arneu et al., Methods in Molecular Biology, 2008, Volume 421, II, 229-243). If the His-Tag at its C-terminus is followed by a Glutamine and an excess of the Qcyclase™ enzyme is present in the reaction in addition to DAPase™, Qcyclase™ catalyzes the formation of a pyroglutamate residue from the glutamine residue at the N-terminus. The pyroglutamate residue is then removed by treatment with pGAPase™ Enzyme. Dipeptides containing pyroglutamate in the N-terminal position cannot serve as DAPase™ substrates and further cleavage is therefore halted. In case the polypeptides or peptides of the present invention comprise a His-tag at their N-terminus and the usage of TAGZyme™ is envisaged, it is preferred to use a His-tag separated by an Glutamine from the following polypeptide or peptide amino acid sequence.

Alternatively a modified version of the His-tag (UZ-HT15: MKHQHQHQHQHQHQQ (SEQ ID NO: 59)) comprising alternating Histidine and Glutamine residues, and custom-modified versions thereof, generated by mutagenesis of the tag may be used in combination with the above-mentioned enzymes TAGZyme™ (DAPase™), Qcyclase™ and pGAPase™ for complete removal of the tag from the polypeptide/precursor peptide of the present invention as described in Arneu et al., Methods in Molecular Biology, 2008, Volume 421, II, 229-243.

### V. Polynucleotides Encoding Polypeptides or Precursor Peptides

A polynucleotide encoding a polypeptide, a precursor peptide or a homologue, variant, fragment or derivative thereof can be composed of any polyribonucleotide or polydeoxyribonucleotide, which may be unmodified RNA or DNA or modified RNA or DNA. For example, a polynucleotide encoding a polypeptide, precursor peptide or a variant, a fragment or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding a polypeptide and/or a precursor peptide of the present invention or a variant, fragment or derivative thereof can be composed of triple-stranded regions comprising RNA or DNA or both RNA and DNA. A polynucleotide encoding a polypeptide and/or a precursor peptide of the present invention as defined hereinabove or a variant, fragment or derivative thereof may also contain one or more modified bases or DNA, or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms.

An isolated polynucleotide encoding a variant, fragment or derivative of a polypeptide and/or a precursor peptide of the present invention as defined hereinabove derived from a polypeptide and/or precursor peptide of the present invention can be created by introducing one or more nucleotide substitutions, additions or deletions into the nucleotide sequence of the polypeptide and/or precursor peptide such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

As is well known, RNA may be isolated from prokaryotic or eukaryotic cells either if the cells were transformed or not transformed by standard techniques, such as a guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art. In one embodiment, cDNAs that encode a polypeptide and/or precursor peptide of the present invention may be generated, either simultaneously or separately, using reverse transcriptase and DNA polymerase in accordance with well known methods. PCR may be initiated by consensus constant region primers, by degenerated or by more specific primers based on the sequence of metagenomic DNA and amino acid sequences of the polypeptides and peptides isolated from the massive, multispecies communities which are part of the animal tissues of organisms such as the sponge Theonella swinhoei, e.g., the sponge itself, and its endobionts such as its as-yet unculturable symbiotic bacteria, (Piel et al., Proc. Natl. Acad. Sci. U.S.A. 101 (2004), 16222-16227; Fisch et al., Nat. Chem. Biol. 5 (2009), 494-501; Nguyen et al., Nat. Biotechnol. 26 (2008), 225-233; Taylor et al., Microbiol. Mol. Biol. Rev. 71 (2007), 295-347). As discussed above, PCR also may be used to isolate DNA clones encoding the polypeptides and/or precursor peptides of the invention, or homologues thereof. In this case the libraries may be screened by consensus primers or larger homologous probes, such as the polynucleotide sequences of the present invention or fragments or derivates thereof as defined hereinabove. Concerning the generation of DNA-libraries from usually quite instable metagenomic marine sponge DNA methods have been developed allowing the generation of libraries comprising 500.000-1.000.000 clones, which is sufficient for the isolation of a gene cluster from the sponges (Fisch et al., Nat. Chem. Biol. 5 (2009), 494-501; Piel et al., Proc. Natl. Acad. Sci. U.S.A. 101 (2004), 16222-16227). Bacteria comprising the individual clones are cultivated in separated colonies in a viscose, three-dimensional medium. Pools of colonies may be screened subsequently by PCR for the presence of particular sequences allowing fast identification and isolation of genes and gene clusters of interest (Gurgui and Piel, Methods in Molecular Biology, 668 (2010) 247-264; Hrvatin and Piel, J. Microbiol. Methods 68 (2007), 434-436)

Plasmid DNA may be isolated from the cells using techniques known in the art, restriction mapped and sequenced in accordance with standard, well known techniques set forth in detail, e.g., in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998) relating to recombinant DNA techniques. Of course, the DNA may be synthetic according to the present invention at any point during the isolation process or subsequent analysis.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2 (1981), 482-489, to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In a preferred embodiment of the present invention, the polynucleotide comprises, consists essentially of, or consists of a nucleic acid having a polynucleotide sequence of the genes of the poy-cluster as depicted in Table 1 and represented by SEQ ID NOs: 1, 2, 4, 6, 8, 10, 12, 14, 16, 18, 20 or 22.

The present invention also includes fragments of the polynucleotides of the invention, as described elsewhere. Additionally polynucleotides which encode fusion polynucleotides, fragments, and other derivatives, as described herein, are also contemplated by the invention.

The polynucleotides may be produced or manufactured by any method known in the art. For example, if the nucleotide sequence encoding a polypeptide and/or precursor peptide is known, a polynucleotide encoding the polypeptide and/or precursor peptide may be assembled from chemically synthesized oligonucleotides, e.g., as described in Kuetmeier et al., BioTechniques 17 (1994) 242-246, which, briefly, involves the synthesis of overlapping oligonucleotides containing portions of the sequence encoding the polypeptide and/or precursor peptide, annealing and ligating of those oligonucleotides, and then amplification of the ligated oligonucleotides by PCR.

Alternatively, a polynucleotide encoding a polypeptide and/or precursor peptide or a fragment, variant, or derivative thereof may be generated from a nucleic acid from a suitable source. If a transformed organism or cell clone containing a nucleic acid encoding a particular polypeptide and/or precursor peptide is not available, but the sequence of the polypeptide and/or precursor peptide is known, a nucleic acid encoding the polypeptide and/or precursor peptide may be chemically synthesized or obtained from a suitable source (e.g., an organism specific or metagenome specific cDNA library, or a cDNA library generated from, or nucleic acid, preferably RNA, isolated from, any group of genetically heterogeneous organisms such as multispecies communities present in the animal tissues such as T. swinhoei, genetically homogenous organisms such as isolated bacterial strains, organisms, cells or tissue expressing the polypeptide and/or precursor peptide, such as transformed bacteria selected to express an polypeptide and/or precursor peptide of the present invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide and/or precursor peptide. Amplified nucleic acids generated by PCR may be cloned then into replicable cloning vectors using any method well known in the art. For the identification and cloning of the poy-locus, the 60000 clone library from PNAS 2004 101 (46) 16222-16227 may be utilized by the use of methods described in Piel, J., Proc. Natl. Acad. Sci. USA 99 (2002), 14002-14007, in particular in methods and materials section at pages: 14002-14005, the disclosure content of which is incorporated hereby by reference; for oligonucleotide sequences see Table 2 below.

Once the nucleotide sequence and corresponding amino acid sequence of the polypeptide and/or precursor peptide, or a fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate polypeptides and/or precursor peptides having a different amino acid sequence, for example to create amino acid substitutions, deletions, and/or insertions.

The sequences of all nucleotide molecules, polypeptides and peptides designated by the SEQ ID NOs identifier hereinbelow, may be found in Tables 1 and 2.

The present invention generally relates to a nucleic acid molecule or a composition of nucleic acid molecules comprising:
(a) a nucleotide sequence of SEQ ID NO: 1;
(b) a nucleotide sequence capable of hybridizing to the nucleotide sequence of SEQ ID NO: 1 under stringent hybridization conditions and encoding at least one polypeptide which catalyzes at least one step of the biosynthesis of polytheonamides and/or encoding a precursor peptide thereof;
(c) a nucleotide sequence encoding at least one polypeptide or peptide selected from any one of poyA (SEQ ID NO: 3), poyB (SEQ ID NO: 5), poyC (SEQ ID NO: 7), poyD (SEQ ID NO: 9), poyE (SEQ ID NO: 11), poyF (SEQ ID NO: 13), poyG (SEQ ID NO: 15), poyH (SEQ ID NO: 17), poyI (SEQ ID NO: 19), poyK (SEQ ID NO: 21) and/or poyJ (SEQ ID NO: 23);
(d) a nucleotide sequence encoding at least one polypeptide which catalyzes at least one step of the biosynthesis of polytheonamides and/or encoding a precursor peptide thereof which amino acid sequence is modified compared to the amino acid sequence of any one of poyA (SEQ ID NO: 3), poyB (SEQ ID NO: 5), poyC (SEQ ID NO: 7), poyD (SEQ ID NO: 9), poyE (SEQ ID NO: 11), poyF (SEQ ID NO: 13), poyG (SEQ ID NO: 15), poyH (SEQ ID NO: 17), poyI (SEQ ID NO: 19), poyK (SEQ ID NO: 21) and/or poyJ (SEQ ID NO: 23) by way of one or more amino acid substitution(s), deletion(s) and/or insertion(s);
(e) a variant or portion of a nucleotide sequence of any one of (a) to (d) encoding at least one polypeptide which catalyzes at least one step of the biosynthesis of polytheonamides and/or encoding a precursor peptide thereof;
(f) a nucleotide sequence which is degenerated with respected to the nucleotide sequence of any one of (a) to (e); or
(g) a nucleotide sequence which is complementary to the nucleotide sequence in any one of (a) to (f).

In one embodiment of the present invention a nucleic acid molecule or a composition of nucleic acid molecules is provided, wherein the nucleotide sequence(s) comprise(s) at least the coding region for any one of poyA (SEQ ID NO: 2 or 90), poyB (SEQ ID NO: 4), poyC (SEQ ID NO: 6), poyD (SEQ ID NO: 8), poyE (SEQ ID NO: 10 or 92), poyF (SEQ ID NO: 12), poyG (SEQ ID NO: 14), poyH (SEQ ID NO: 16), poyI (SEQ ID NO: 18), poyK (SEQ ID NO: 20) and/or poyJ (SEQ ID NO: 22), including variants or portions thereof, wherein the variants or portions encode a polypeptide which retains the biological activity of the respective polypeptide.

In one preferred embodiment the present invention provides a nucleic acid molecule as defined above, wherein the nucleotide sequence differs in at least one nucleotide from the nucleotide sequence represented by SEQ ID NOs: 1, 2, 4, 6, 8, 10, 12, 14, 16, 18, 20, 22, 90 or 92.

Furthermore, in one embodiment of the present invention the nucleic acid molecules of the present invention as defined above encode polypeptides, wherein the encoded polypeptide and/or peptide differs in at least one amino acid from the amino acid sequence of SEQ ID NOs: 3, 5, 7, 9, 11, 13, 15, 17, 19, 21 or 23.

It is one achievement of the present invention to provide nucleic acid molecules encoding polypeptides involved in at least one step of the biosynthesis of polytheonamides and/or the precursor peptide thereof. For the production of the corresponding polypeptides, said nucleic acid molecules of sequences as defined by SEQ ID NOs: 2, 4, 6, 8, 10, 12, 14, 16, 18, 20, 22, 90 or 92, enlisted in Table 1 and described above may be used for the construction of nucleic acid molecules comprising additional sequences such as recognition sequences of DNA modifying enzymes, e.g., of endodeoxyribonucleases (restriction endonucleases; restriction enzymes) or sequences which by being operably linked to those influence the expression of the nucleic acid sequences of the present invention, wherein the term expression is used herein in respect of both, the transcription of the nucleic acid sequences into mRNA molecules and/or the following translation into polypeptides and proteins. By this definition sequences which influence the transcription, the posttranscriptional processing, the lifetime and/or the translation of the transcriptional products of said nucleic acid molecules are included as well.

Thus, in one embodiment the present invention provides one or more nucleic acid molecules of a sequence as defined hereinabove, wherein the polypeptide and/or peptide encoding nucleotide sequences are operatively linked to at least one expression control sequence. Examples of such expression control sequences are promoter, operator, enhancer, silencer sequences, transcription terminators, polyadenylation sites and other nucleic acid sequences known in the art which may be used for the expression of the polypeptides and/or peptides of the present invention.

Said expression control sequences may enhance or downregulate the expression levels of the polypeptide and/or peptide encoding nucleotide sequences operatively linked to. One or several expression control sequences may be used in combination with each other and/or in combination with one or more of the polypeptide and/or peptide encoding nucleotide sequences as defined in the present invention depending on the cell type (e.g., prokaryotes or eukaryotes) or organism used for the expression of the polypeptide and/or peptide encoding nucleotide sequences. The expression regulatory sequences may be chosen as well in respect of the time (i.e. developmental stage), cell type and/or general circumstances, e.g., the presence and/or absence of one or more specific substances, wherein the polypeptides and/or peptides encoded by the nucleotide sequences as defined hereinabove are expressed when said regulatory sequences operably linked to the polypeptide and/or peptide encoding nucleotide sequences permit their expression because one or more of the mentioned conditions are met or not expressed, when the circumstances permitting expression are not met.

The expression control sequences used may originate from the same organism as the polypeptide and/or peptide encoding nucleotide sequences of the present invention as defined hereinabove or they may be foreign, i.e. originate from another organism in the meaning of different taxonomy or phylogeny. In one embodiment the present invention provides a nucleic acid molecule comprising the polypeptide and/or peptide encoding nucleotide sequences, wherein at least one expression control sequence is foreign to the polypeptide and/or peptide encoding nucleotide sequences.

The polynucleotide as employed in accordance with this invention and encoding the above described polypeptides involved in the biosynthesis of polytheonamides or the precursor peptide thereof may be, e.g., DNA, cDNA, RNA or synthetically produced DNA or RNA or a recombinantly produced chimeric nucleic acid molecule comprising any of those polynucleotides either alone or in combination. Preferably, the polynucleotides are operatively linked to expression control sequences allowing expression in prokaryotic or eukaryotic cells. Expression of said polynucleotide comprises transcription of the polynucleotide into a translatable mRNA. Details describing the expression of the polynucleotides of the present invention will be described further below in this description.

The nucleotide sequence(s) depicted in SEQ ID NOs: 1, 2, 4, 6, 8, 10, 12, 14, 16, 18, 20, 22, 90 or 92 and enlisted in Table 1 encode(s) at least one polypeptide which catalyzes at least one step of the biosynthesis of polytheonamides and/or a precursor peptide thereof. Polytheonamides are a novel class of proteins which were previously described in Hamada et al, Tetrahedron Lett. 35 (1994) 719-720, Hamada et al., J. Am. Chem. Soc. 127 (2005), 110-118, Hamada et al., J. Am. Chem. Soc. 132 (2010), 12941-12945, Iwamoto et al., FEBS Lett. 584 (2010), 3995-3999. Neither the gene encoding the precursor peptide, nor mechanisms or enzymes involved in the production of polytheonamides were known before. By the provision of the nucleotide sequences of nucleic acid molecules of the present invention it is now possible to isolate identical or similar polynucleotides which encode for polypeptides or proteins capable of catalyzing at least one step of the biosynthesis of polytheonamides and/or a precursor peptide thereof from other species or organisms. Said nucleotide sequences may be employed, in accordance with this invention, in the production/preparation of polypeptides involved in at least one step of the biosynthetical processes and methods for the generation of polytheonamides and/or the precursor thereof and/or for preparing pharmaceutical compositions comprising peptide-based compound(s) produced by these methods wherein at least one step of the biosynthetical processes has been performed as well as related pharmaceutical uses and/or methods described herein. Well-established approaches for the identification and isolation of such related nucleotide sequences are, for example, the isolation from genomic or cDNA libraries using the complete or part of the disclosed sequence as a probe or the amplification of corresponding polynucleotides by polymerase chain reaction using specific primers.

Thus, in one embodiment the invention also relates to nucleic acid molecule(s) capable of specifically hybridizing to a nucleic acid molecule as described above under stringent hybridization conditions. The invention further relates to nucleic acid molecule(s) capable of specifically hybridizing to a nucleic acid molecule as described above and differing in one or more positions in comparison to these as long as they encode a polypeptide involved in at least one step in the process of biosynthesis of polytheonamides or the precursor peptide thereof as defined above. Such molecules comprise those which are changed, for example, by deletion(s), insertion(s), alteration(s) or any other modification known in the art in comparison to the above described polynucleotides either alone or in combination. Methods for introducing such modifications in the polynucleotides of the invention are well-known to the person skilled in the art; see, e.g., Sambrook et al. (Molecular Cloning; A Laboratory Manual, Second Edition, Cold Spring Harbor Laboratory Press, Cold Spring Harbor N.Y. (1989)) and Ausubel, Current Protocols in Molecular Biology, Green Publishing Associates and Wiley Interscience, N.Y. (1994). The invention also relates to polynucleotides with a nucleotide sequence different from the nucleotide sequence of any of the above-described polynucleotides due to the degeneracy of the genetic code.

Thus, the invention also relates to polynucleotides which hybridize to the above-described polynucleotides and differ at one or more positions in comparison to these as long as they encode a polypeptide as defined above by its involvement in the biosynthesis posttranslational process of polytheonamide biosynthesis and/or by its biological activity as identified above. Such molecules comprise those which are changed, for example, by deletion(s), insertion(s), alteration(s) or any other modification known in the art in comparison to the above described polynucleotides either alone or in combination. Methods for introducing such modifications in the polynucleotides of the invention are well-known to the person skilled in the art; see, e.g., Sambrook et al. (Molecular cloning; A Laboratory Manual, Second Edition, Cold Spring Harbor Laboratory Press, Cold Spring Harbor N.Y. (1989)). The invention also relates to polynucleotides the nucleotide sequence of which differs from the nucleotide sequence of any of the above-described polynucleotides due to the degeneracy of the genetic code.

With respect to the DNA sequences characterized under (iv) above, the term “hybridizing” in this context is understood as referring to conventional hybridization conditions, preferably such as hybridization in 50% formamide/6×SSC/0.1% SDS/100 μg/ml ssDNA (ss=single strand), in which temperatures for hybridization are above 37° C. and temperatures for washing in 0.1×SSC/0.1% SDS are above 55° C. Most preferably, the term “hybridizing” refers to stringent hybridization conditions, for example such as described in Sambrook, supra.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. In general, “stringent hybridization” is performed at about 25° C. below the thermal melting point (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., pages 9.50-9.51, hereby incorporated by reference.

The Tm for a particular DNA-DNA hybrid can be estimated by the formula:

Tm=81.5° C.−16.6(log₁₀[Na⁺])+0.41(% fraction G+C)−0.63(% formamide)−(600/L)

where L is the length of the hybrid in base pairs. This equation is valid for concentrations of Na⁺ of 0.01M to 0.4M (and less accurately for higher Na⁺ concentrations) and for DNAs whose G+C content is in the range of 30-75%.

The Tm for a particular RNA-RNA hybrid can be estimated by the formula:

Tm=79.8° C.+18.5(log₁₀[Na⁺])+0.58(% fraction G+C)+11.8(% fraction G+C)²−0.35(% formamide)−(820/L).

The Tm for a particular RNA-DNA hybrid can be estimated by the formula:

Tm=79.8° C.+18.5(log₁₀[Na⁺])0.58(% fraction G+C)+11.8(fraction G+C)²−0.50(% formamide)−(820/L).

The above equations apply only to hybrids longer than 100 nucleotides.

In general, the Tm decreases by 1-1.5° C. for each 1% of mismatch between two nucleic acid sequences. Thus, one who is having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tm of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well known in the art.

An example of stringent hybridization conditions for hybridization of complementary nucleic acid sequences having more than 100 complementary residues on a filter in a Southern or Northern blot or for screening a library is 50% formamide/6×SSC at 42° C. for at least ten hours. Another example of stringent hybridization conditions is 6×SSC at 68° C. for at least ten hours. An example of low stringency hybridization conditions for hybridization of complementary nucleic acid sequences having more than 100 complementary residues on a filter in a Southern or northern blot or for screening a library is 6×SSC at 42° C. for at least ten hours. Hybridization conditions to identify nucleic acid sequences that are similar but not identical can be identified by experimentally changing the hybridization temperature from 68° C. to 42° C. while keeping the salt concentration constant (6×SSC), or keeping the hybridization temperature and salt concentration constant (e.g. 42° C. and 6×SSC) and varying the formamide concentration from 50% to 0%. Hybridization buffers may also include blocking agents to lower background. These agents are well-known in the art. See Sambrook et al., pages 8.46 and 9.46-9.58, herein incorporated by reference.

Washing conditions can be altered to change stringency conditions as well. An example of stringent washing conditions is a 0.2×SSC wash at 65° C. for 15 minutes (see Sambrook et al., for SSC buffer). Often the high stringency wash is preceded by a low stringency wash to remove excess probe. An exemplary medium stringency wash for duplex DNA of more than 100 base pairs is 1×SSC at 45° C. for 15 minutes. An exemplary low stringency wash for such a duplex is 4×SSC at 40° C. for 15 minutes. In general, signal-to-noise ratio of 2-fold or higher than that observed for an unrelated probe in the particular hybridization assay indicates detection of a specific hybridization.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using NCBI BLASTx and BLASTn software. Alternatively, Fasta, a program in GCG Version 6.1 may be used. Fasta provides alignments and percent sequence identity of the regions of the best overlap between the query and search sequences (Pearson, 1990, herein incorporated by reference). For instance, percent sequence identity between nucleic acid sequences can be determined using Fasta with its default parameters (a word size of 6 and the NOPAM factor for the scoring matrix) as provided in GCG Version 6.1, herein incorporated by reference.

Particularly preferred are polynucleotides which share 70%, preferably at least 85%, more preferably 90-95%, and most preferably 96-99% sequence identity with one of the above-mentioned polynucleotides and have the same biological activity. Such polynucleotides also comprise those which are altered, for example by nucleotide deletion(s), insertion(s), substitution(s), addition(s), and/or recombination(s) and/or any other modification(s) known in the art either alone or in combination in comparison to the above-described polynucleotides. Methods for introducing such modifications in the nucleotide sequence of the polynucleotide of the invention are well known to the person skilled in the art. Thus, the present invention encompasses any polynucleotide that can be derived from the above-described polynucleotides by way of genetic engineering and that encode upon expression a polypeptide or protein or a biologically active fragment thereof retaining the biological activity of catalyzing at least one step of the biosynthesis of polytheonamides or a nucleic acid molecule encoding a precursor peptide thereof.

The nucleic acid molecule or polynucleotide of the present invention can be composed of any polyribonucleotide or polydeoxyribonucleotide, which may be unmodified RNA or DNA or modified RNA or DNA. For example, polynucleotides according to the present invention can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides of the present invention can be composed of triple-stranded regions comprising RNA or DNA or both RNA and DNA. The polynucleotides of the present invention may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

Particularly preferred are polynucleotides which share at least 70%, preferably at least 85%, more preferably 90-95%, and most preferably 96-99% sequence identity with one of the above-mentioned polynucleotides having the nucleotide sequence represented by SEQ ID NOs: 1, 2, 4, 6, 8, 10, 12, 14, 16, 18, 20, 22, 90 or 92 and encoding polypeptides which have the same biological activity. Such polynucleotides also comprise those which are altered, for example by nucleotide deletion(s), insertion(s), substitution(s), addition(s), and/or recombination(s) and/or any other modification(s) known in the art either alone or in combination in comparison to the above-described polynucleotides. Methods for introducing such modifications in the nucleotide sequence of the polynucleotide of the invention are well known to the person skilled in the art. Thus, the pharmaceutical composition(s), use(s) and method(s) of the present invention may comprise any polynucleotide that can be derived from the above described polynucleotides by way of genetic engineering and that encode upon expression a polypeptide, protein or a biologically active fragment thereof capable of catalyzing at least one step in the polytheonamide biosynthesis.

As known in the art, “sequence identity” between two polypeptides or two polynucleotides is determined by comparing the amino acid or nucleic acid sequence of one polypeptide or polynucleotide to the sequence of a second polypeptide or polynucleotide. As a practical matter, whether any particular polypeptide is at least 40%, 50%, 60%, 70%, 80%; 90%, 95%, 96%, 97%, 98% or 99% identical to one of the amino acid sequences shown in SEQ ID NOs: 3, 5, 7, 9, 11, 13, 15, 17, 19, 21 or 23 can be determined conventionally using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2 (1981), 482-489, to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

Naturally occurring variants of polynucleotides are called “allelic variants” or “alleles” and refer to one of several alternate forms by means of base sequence alterations of a gene occupying a given locus on a chromosome of an organism which may also result in an alternate form of the corresponding polypeptide encoded by the given gene (Genes II, Lewin, B., ed., John Wiley & Sons, New York (1985) and updated versions). Alternatively, non-naturally occurring variants may be produced by mutagenesis techniques or by direct synthesis.

It is also immediately evident to the person skilled in the art that regulatory sequences may be added to the polynucleotide(s) as defined hereinabove and employed in the pharmaceutical composition, uses and/or methods of the invention. For example, promoters, transcriptional enhancers and/or sequences which allow for induced expression of the polynucleotide of the invention may be employed. A suitable inducible system is, for example, tetracycline-regulated gene expression as described, e.g., by Gossen and Bujard, Proc. Natl. Acad. Sci. USA 89 (1992), 5547-5551, Gossen et al., Trends Biotech. 12 (1994), 58-62 and Bello et al., Development 125 (1998), 2193-2202. A further example of a suitable system is the GAL4/UAS system for the regulation of gene expression as described, e.g., by Brand and Perrimon, Development 118 (1993), 401-415, extension of this system by GAL80, a repressor of GAL4, (Ma and Ptashne, Cell 50 (1987), 137-142; Salmeron et al., Genetics 125 (1990), 21-27) or thermo-sensitive (GAL80ts) forms of it (McGuire et al., Science 302 (2003), 1765-1768) reviewed in Duffy, Genesis 34 (2002), 1-15.

### VI. Oligonucleotides

In one embodiment, the invention provides a pair of nucleic acid molecules which correspond to the 5′ and reverse complement of the 3′ end of a nucleotide sequence of the nucleic acid molecule as described hereinabove. This pair of nucleic acid molecules of at least 15 nucleotides in length hybridizes specifically with a polynucleotide as described above or with a complementary strand thereof. Preferred are nucleic acid probes of 17 to 35 nucleotides in length. Of course, it may also be appropriate to use nucleic acids of up to 100 and more nucleotides in length. Said nucleic acid probes are particularly useful for various biotechnological and/or screening applications. On the one hand, they may be used as PCR primers for amplification of polynucleotides encoding polypeptides and peptides of the present application and/or their homologues and may, thereby, serve as useful biotechnological tools. Another application is the use as a hybridization probe to identify polynucleotides hybridizing to the polynucleotides encoding the polypeptides of the present invention capable of catalyzing at least one step in the biosynthesis of polytheonamides as defined hereinabove and precursor peptides thereof by homology screening of genomic DNA libraries. Furthermore, the person skilled in the art is well aware that it is also possible to label such a nucleic acid probe with an appropriate marker for specific applications, such as for the detection of the presence of a polynucleotide as described herein above in a sample derived from an organism. The above mentioned nucleic acid molecules may either be DNA or RNA or a hybrid thereof.

In this respect, it is also to be understood that the polynucleotide to be used in the invention can be employed for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press.

### VII. Vectors and Regulatory Elements

Preferably, the nucleic acid molecule of the present invention as defined hereinabove is comprised in a vector. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode a polypeptide capable of catalyzing at least one step in the biosynthesis of polytheonamides and a precursor peptide or another peptide intended to be subdued such an catalyzing step. In addition, a vector, polynucleotide, or nucleic acid of the invention may encode heterologous coding regions, either fused or unfused to a nucleic acid encoding a binding molecule, an antibody, or fragment, variant, or derivative thereof. Heterologous coding regions include without limitation specialized elements or motifs, such as a secretory signal peptide or a heterologous functional domain. Such vectors may comprise further genes such as marker genes which allow for the selection of said vector in a suitable host cell and under suitable conditions. Preferably, the polynucleotides are operatively linked to expression control sequences allowing expression in prokaryotic or eukaryotic cells. Expression of said polynucleotide(s) comprises transcription of the polynucleotide(s) into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in prokaryotic host cells comprise, e.g., the P_{L} (Phage lamda), Phage T5, lac, T7, trp or tac promoter in bacterial hosts, e.g., E. coli. In other bacteria strains, other promoters may be used, such as the xylose-operon (Rygus et al., Arch Microbiol. 155 (1991), 535-542) in B. megaterium; P43-promoter (Daguer et al., Lett. Appl. Microbiol. 41 (2005), 221-226), vegI-promoter (Lam et al., J. Biotechnol. 63, (1998), 167-177), xylose-inducible promoter (Kim et al. Gene 181, (1996), 71-76) and the tet-inducible promoter (Geissendoerfer and Hillen, Appl. Microbiol. Biotechnol. 33 (1990), 657-663) in B. subtilis; lac-promoter system in Caulobacter crescentus (Umelo-Njaka et al., Plasmid 46 (2001), 37-46). Examples for regulatory elements permitting expression in eukaryotic host cells are the AOX1 or GAL1 promoter in yeast, the UAS-promoter with sequences encoding the GAL4-activator and/or GAL80-repressor or an AcNPV promoter such as the polyhedron promoter in insect cells or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used, leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. Concerning the expression of precursor peptides of the present invention, the N-terminal addition of a tag is preferred, because of the possibility of tag-removal by exonuclease treatment and because C-terminal tags might be modified by the polytheonamide enzymes of the present invention. In this context, suitable expression vectors are known in the art such as Okayama-Berg cDNA expression vector pcDV1 (Pharmacia), pCDM8, pRc/CMV, pcDNA1, pcDNA3 (Invitrogene), or pSPORT1 (GIBCO BRL), pET-vectors (Novagen, Inc.), pCDF-vectors (Novagen Inc.), pUC-vectors (e.g., pUC18, pUC19; University of California), pBR322-vectors, pBluescript and pBluescript II-Vectors (Stratagene) and modified versions thereof as described in Sambrook, J., Fritsch, E. F., and Maniatis, T. (1989). Molecular Cloning: A Laboratory Manual, Second Edition (Plainview, N.Y.: Cold Spring Harbor Laboratory Press) or in Ausubel, F. M., Brent, R., Kingston, R. E., Moore, D. D., Seidman, J. G., Smith, J. A., and Struhl, K. (1994). Current Protocols in Molecular Biology (New York: Greene Publishing Associates and Wiley-Interscience). Vectors suitable for the addition of tags are also known in the art such as vectors pET28a-c or pHis-8 for the N-terminal addition of a His-tag and (pET41a) for the N-terminal addition of the GST-tag to a peptide or polypeptide of interest.

Preferably, the expression control sequences will be prokaryotic promoter systems in vectors capable of transforming or transfecting prokaryotic host cells, but control sequences for eukaryotic hosts may also be used. Due to the necessity to transfect and to maintain more than one vector at once into one cell, plasmids with compatible origins of replication and independent antibiotic selection are required. In this respect many vectors may be used, a comprehensive list of contemporary available vectors, their selection markers and compatibility information concerning their origins of replication may be found on page 58 in Table 1 of Tolia and Joshua-Tor, Nat. Methods 3 (2006), 55-64. In a preferred embodiment of the present invention Duet vectors, such as pETDuet™ and pCDFDuet™ vectors (trademarks of Merck KGaA, Darmstadt, Germany) carrying compatible replicons and antibiotic resistance markers (pETDuet™-vector comprising the bla-marker for ampicillin or carbenicillin resistance; pCDFDuet™-vector comprising aadA-marker for streptomycin or spectinomycin resistance) are used together in appropriate host strains to coexpress up to eight proteins as described in detail in Examples 1, 3 and 4 below.

### VIII. Host Cells

Once the appropriate genetic material is obtained and, if desired, modified to encode an modified version of the polypeptide or precursor peptide, the coding sequences can be inserted into expression systems contained on vectors which can be transfected into standard recombinant host cells. In this respect, is self-evident that in one embodiment the present invention also provides a host cell comprising a vector comprising as defined hereinabove. Said vector may comprise one or more of the genes/polynucleotides as defined hereinabove and also the host may comprise one or more of the vectors. As well, according to the present invention a host may comprise one or more vectors comprising polynucleotides encoding one or more of the polypeptides of the present invention including also a precursor peptide or protein of a polytheonamide. In one embodiment of the present invention the host cell of the present invention comprises a gene encoding a selected precursor peptide or protein, which is not encoded by a nucleic acid molecule as defined hereinabove. In this respect the host cell may comprise a gene encoding any selected precursor peptide or protein, however, in a preferred embodiment of the present invention the selected precursor peptide or protein is selected from the group defined herewith by the term “proteusins” and consisting of precursors of polytheonamides and other members of the nitrile hydratase leader peptide family (NHLP; Haft et al., BMC Biol. 8:70 (2010))

A variety of such host cells may be used; for efficient processing, however, in one preferred embodiment of the present invention the host cell used by the methods of the present invention is a microorganism. In a particularly preferred embodiment of the present invention a host cell is provided, which is a bacterial host. However, eukaryotic and mammalian expression systems may be used as well in accordance with the methods of the present invention. Typical mammalian cell lines useful for this purpose include, but are not limited to, CHO cells, HEK 293 cells, or NSO cells. Host cells, such as bacteria, fungi, plants or cell lines are available commercially or may be obtained from different cell culture collections, such as ATCC.

In this respect, different promoter systems and bacterial hosts for the expression of the peptides and/or polypeptides of the present invention may be chosen. As hosts, e.g., members of the Enterobacteriaceae, such as strains of Escherichia coli (e.g., E. coli strains BL21 or K12) or Salmonella typhimurium; Bacillaceae, such as Bacillus subtilis, Bacillus amyloliquefaciens, Aneurinibacillus migulanus (formerly known as Bacillus brevis) and Bacillus megaterium; Caulobacteraceae such as Caulobacter crescentus; Pasteurellaceae such as Haemophilus influenza; Pseudomonadaceae such as Pseudomonas putida; Streptococcaceae such as Streptococcus pneumoniae also known as Pneumococcus; may be used. In one embodiment of the present invention E. coli strain BL21 and its derivatives are used. In a preferred embodiment the BL21-strains derivatives such as the arabinose-inducible E. coli strain Bl21(DE3)AI™ (Life Technologies Corporation/Invitrogen; Genotype: F ompT hsdSB(r_{B}⁻m_{B}⁻) gal dcm araB::T7RNAP-tetA; carries the gene for the T7 RNA polymerase under the control of the arabinose inducible araB-promoter) and the IPTG-inducible Bl21Star™ (DE3)pLysS strain (Invitrogen Catalog No: C6020-03; Genotype: F⁻ ompT hsdSB (r_{B}⁻m_{B}⁻) gal dcm rne131 (DE3) pLysS (CamR); the strain contains the DE3 lysogen that carries the gene for T7 RNA polymerase under control of the IPTG inducible lacUV5 promoter) are used.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris. For expression in Saccharomyces, the plasmid YRp7, for example, (Stinchcomb et al., Nature 282 (1979), 39-43; Kingsman et al., Gene 7 (1979), 141-152; Tschemper et al., Gene 10 (1980), 157-166) is commonly used. This plasmid already contains the TRP1 gene which provides a selection marker for a mutant strain of yeast lacking the ability to grow in tryptophan, for example ATCC No. 44076 or PEP4-1 (Jones, Genetics 85 (1977), 23-33). The presence of the trp1 lesion as a characteristic of the yeast host cell genome then provides an effective environment for detecting transformation by growth in the absence of tryptophan.

Furthermore, insect cells may also be used for the expression of the polypeptides and precursor peptides of the present invention. Insect cells are available commercially (e.g., Expression Systems, LLC, USA) or from culture collections such as ATCC, otherwise they may be developed as described in Lynn, Cytotechnology 20 (1996), 3-11.

In this respect, plasmid or virus based vector systems may be used to introduce into and express the polynucleotides of the present invention in insect cells. Many types of viruses infect insects, however, viruses belonging to the family of Baculoviridae are mostly used in the art due to their capability of infecting over 500 species of insects (Granados and McKenna, 1995. “Insect Cell Culture Methods and Their Use in Virus Research”. In: Schuler and Wood, Granados R R, Hammer D A, editors. Baculovirus Expression Systems and Biopesticides p. 13-39. New York: Wiley-Liss). Typically, an expression vector system based on the baculovirus Autographa californica nuclear polyhedrosis (AcNPV) is used in insect cells as a vector for foreign genes expression (Smith et al., Journal of Virology 46 (1983), 584-593). The original baculovirus replicates in the nucleus of over 30 lepidopteran insect cell lines and the expression vector system based on it may be used for the expression of genes originating from all types of organisms such as viruses, fungi, bacteria, plants and animals. Over 500 continuous cell lines have been established from over 100 insect species (Lynn, Methods in Cell Science 21 (1999), 173-181), however, most widely used are Spodoptera frugiperda cells, such as Sf9 and Sf21 cell lines; Trichoplisia ni BTI-Tn-5B1-4 (High Five) or Tn 368 cell lines; and Drosophila S2 cells. The polypeptide or precursor peptide encoding sequences of the present invention may be cloned individually into non-essential regions (for example the polyhedrin gene) of the virus and placed under control of an AcNPV promoter (for example the polyhedrin promoter) and expressed in the above-mentioned insect cells. Polynucleotides encoding different polypeptides and/or peptide precursors may be fused comprising IRES sequences in-between the different polynucleotides and placed under control of a promoter, such as the AcNPV promoter permitting thereby the expression of several polypeptides and/or peptide precursors in a single transfected cell.

Furthermore, plasmid vectors may be used for transient or stable transfection and expression of the polypeptides and the precursor peptides of the present invention. For example, methods may be used which base on the transfection of a composition of plasmids comprising several polynucleotides inducible by the same inductors and encoding after induction a selection marker and the genes of interest. By addition or expression of an adequate inductor, transcription of the polynucleotides starts and cells expressing them may be selected by the presence of the selection marker, wherein prolonged culturing in a selective medium permits the establishment of stable transfected cell lines and the production of the polypeptides of interest, such as the polypeptdides and/or peptide precursors of the present invention. Such methods are known in the art and are described, e.g., in Makridou et al., Genesis, 36 (2003), 83-7 for the use of S2-cells and the expression of up to four different proteins under control of the UAS-GAL4 system, which may be expressing constitutively or inducible, depending on the construction of the system, e.g., by the use of inducible promoters, such as the tet, or the metallothionein promoter for the expression of the GAL4-transcriptional activator, GAL4-hormone receptor fusions (Duffy, Genesis 34 (2002), 1-15). General methods for cell culture of insect cells are known in the art and described for example in Lynn, (2002), J Insect Sci. (2002); 2:9. Epub.

Promoters which may be used according to the present invention are promoters which base on the main features of the 1-arabinose inducible araBAD promoter (PBAD), the lac promoter, the 1-rhamnose inducible rhaP BAD promoter, the T7 RNA polymerase promoter, the trc and tac promoter, the lambda phage promoter p L, and the tetracycline-inducible tetA promoter/operator, for example. In a preferred embodiment of the present invention T7lac promoter are used for protein expression The T7 expression system host strains (DE3 lysogens) are covered by U.S. Pat. No. 5,693,489

### IX. Production and Purification of the Polypeptides, Peptide Precursors and Peptide-Like Compounds of the Present Invention

In one embodiment of the present invention the polypeptides and/or precursor peptides of the present invention are provided as described in the Examples below by a method for preparing at least one polypeptide which catalyzes at least one step of the biosynthesis of polytheonamides and/or for preparing a precursor peptide thereof, said method comprising
(a) culturing the host cell comprising a nucleic acid or a composition of nucleic acids of the present invention under conditions allowing the expression of the nucleic acid molecule; and optionally
(b) recovering the polypeptide(s) and/or the precursor peptide.

The recovery of the polypeptide(s) and/or precursor peptides is performed by isolating them from the culture. The expression systems may be designed to include signal peptides so the resulting polypeptides are secreted into the medium or the periplasmic space; however, intracellular production is also possible. Once a polypeptide or a precursor peptide molecule of the invention has been recombinantly expressed, they can be purified according to standard procedures of the art, including for example, by chromatography (e.g., by ion exchange, affinity purification, and size-exclusion column chromatography), centrifugation, differential solubility, e.g. ammonium sulfate precipitation, or by any other standard technique for the purification of proteins; see, e.g., Scopes, “Protein Purification”, Springer Verlag, N.Y. (1982). Particularly preferred are purifications by affinity of the His-Tag for metal ions, such as nickel, cobalt or zinc ions, immobilized on a chromatographic support as appropriate matrix, such as nitriloacetate. Preferably Ni-NTA agarose (NTA=nitrilotriacetic acid, chelating moiety linking the nickel ions to agarose) may be used for purification of polypeptides or precursor peptides of the present invention comprising His-tags (see Example 5) or glutathione agarose is used for purification of polypeptides or precursor peptides comprising GST-tags, and, if necessary, further purification by chromatographic steps, such as ion exchange, size exclusion or hydrophobic interaction chromatography.

Furthermore, in one embodiment the present invention relates to a composition comprising at least one polypeptide which catalyzes at least one step of the biosynthesis of polytheonamides encoded by the nucleic acid molecule as provided by the present invention and defined hereinabove or produced by the method for preparing at least one polypeptide catalyzing at least one step of the biosynthesis of polytheonamides and/or for preparing a precursor peptide thereof.

In this respect, in one embodiment the present invention relates to a method for preparing a selected peptide-based compound or precursor thereof, said method comprising
(a) culturing the host cell of the present invention as defined hereinabove under conditions under which the cell will produce the polypeptide(s) and a precursor peptide or subjecting a precursor peptide as defined in any one of the preceding claims to a composition comprising at least one polypeptide which catalyzes at least one step of the biosynthesis of polytheonamides; and optionally
(b) recovering the peptide-based compound.

The polynucleotides (nucleic acid molecules) encoding polypeptides of the present invention, which catalyze at least one step of the biosynthesis of polytheonamides and the polynucleotide encoding the precursor peptide thereof have been found in the metagenome of Theonella swinhoei. By means of performing the abovementioned method for preparing a selected peptide-based compound or precursor thereof, the polynucleotides may be introduced, comprised in vectors as described hereinbefore, again into the organisms they originate from, i.e. endobionts/symbionts of T. swinhoei as host cells where their expression may be performed in addition or in replacement of the expression of the endogenous polynucleotides, thus providing a method for preparing one or more of said polypeptides, selected peptide-based compounds and precursors thereof wherein the peptide-based compounds may be polytheonamides and the precursors of the compounds the respective precursors of polytheonamides. However, polynucleotides encoding other peptide-base compounds or precursors thereof which are not endogenous to the metagenome of T. swinhoei may be introduced by analogue means and produced in the endobionts of T. swinhoei as well, and vice versa, the polynucleotides of the present invention comprised in vectors as described hereinabove may be introduced into host cells, different from these the polynucleotides originate from, e.g., into E. coli, be expressed and used therein in the aforementioned method for preparing one or more of said polypeptides, selected peptide-based compounds and/or precursors thereof. Due to the requirement to meet the general demands made on the biotechnological production of polypeptides, peptide-based compounds or peptide analogues, such as requirements concerning feasibility, safety, reliability, yield and cost-effectiveness of their production, in the majority of cases organisms or cells (in cell culture production techniques) have to be chosen in this respect which are different from the organisms the polynucleotides are originating from, host cells and bacterial strains thus as mentioned supra. Therefore, in one embodiment of the present invention it is also an object of the present invention to provide a method for preparing a selected peptide-based compound or precursor thereof, wherein the cell does not produce the peptide-based compound in the absence of the nucleic acid molecule. In this respect, in one preferred embodiment of the present invention a method is provided, wherein the peptide-based compound is a polytheonamide.

As described above, hitherto the provision of polytheonamides by extraction and isolation from sponge specimen was cumbersome, prone to contamination and resulting only in rather minute amounts which made it difficult if not impossible to envisage their use as pharmaceuticals.

In contrast, with the means and methods provided by the present invention it is now possible to produce polytheonamides and like compounds in unlimited amounts and due to the possible selection of appropriate host cell expression systems at a pharmaceutical grade without contamination by, for example, other possible toxic components which are known to reside in invertebrate organisms.

Accordingly, in one further embodiment the present invention relates to peptide based compounds obtainable by any one of the above described methods of the present invention for producing the same and as illustrated in the examples. In this context, the present invention relates to peptide based compounds such as polytheonamides with high purity in terms of weight-% compared to possible contaminations of about 90%, 91%, 92%, 93%, 94%, 95%, 96%, 97%, 98% preferably 99%, 99.1%, 99.2%, 99.3%, 99.4%, 99.5%, 99.6%, 99.7%, 99.8% and most preferably 99.9% or even substantially free from any other components.

By the introduction of the polynucleotides of the present invention encoding one or more of the polypeptides and/or precursors thereof of the present invention into the host cells as defined above, the cells are modified in the meaning of expressing said polypeptides, peptides and precursors of peptide-based compounds in a way which is not natural either in respect of the levels of production or of the kind of said polypeptides, peptides and precursors of peptide-based compounds. The expression levels of the polypeptides and peptides of the present invention and respective biosynthetical products of their biological activity, such as peptide-based compounds are not natural when their homologues or endogenous versions are expressed and synthesised in the host cells as well without introduction of the polynucleotides of the present invention, i.e. in hosts the polynucleotides originate from or in hosts encoding their homologues. The expression levels, encoded polypeptides, peptides and respective biosynthetical products of their biological activity such as peptide-based compounds as described above are not natural in hosts which do not comprise endogenous polynucleotides which are identical or homologue to the polynucleotides of the present invention. Furthermore, peptide-based compounds may be produced according to the present invention, which are not natural to both kinds of hosts. Such non-natural peptide-base compounds may be produced for example, if precursor peptides different, in the meaning of lacking, or having low sequence identity with the precursor peptide PoyA of polytheonamides of the sequence as defined in SEQ ID No: 3, or the cleaved form thereof of the sequence TGIGVVVAVVAGAVANTGAGVNQVAGGNINVVGNINVNANVSVNMNQTT (SEQ ID NO: 48) are expressed and subjected to the activity of one or more of the polypeptides of the present invention capable of catalyzing at least one step of the biosynthesis of polytheonamides. It is understood that in the meaning of the term “precursor peptides different from the precursor peptide of polytheonamides of the sequence as defined in SEQ ID NO: 3” as well fragments, derivates, homologues and mutants of the peptide are included. Furthermore, non-natural peptide-base compounds may be produced according to the methods of the present invention if precursor peptides of polytheonamides of the sequence as defined in SEQ ID NO: 3 are expressed and subjected to the activity of one or more, but not to the activity of all of the polypeptides of the present invention capable of catalyzing at least one step of the biosynthesis of polytheonamides. Non-natural peptide-base compounds may be produced as well when precursor peptides of a sequence completely unrelated to the sequence as defined in SEQ ID NO 3 are expressed and subjected to the activity of one or more, or of all of the polypeptides of the present invention capable of catalyzing at least one step of the biosynthesis of polytheonamides.

Therefore, in one embodiment of the present invention the peptide-based compound obtainable by the aforementioned method for preparing a selected peptide-based compound or precursor thereof is not a natural peptide-based compound. In particular, the present invention may not encompass natural peptide based compounds such as polytheonamide which have been provided by conventional isolation (from the sponge T-swinhoei) means prior to the present invention for example the polytheonamide composition described in Tetrahedron Lett 35 (1994), 719-720.

In a further embodiment of the present invention the peptide-based compound, obtainable by the aforementioned method for preparing a selected peptide-based compound or precursor thereof, is a polytheonamide.

As explained above, the polypeptides or peptide precursors of the present invention may be expressed as fusion proteins with tags adequate for their recognition and/or purification by the use of molecules or polypeptides specifically recognizing and binding said tags, e.g., by specific antibodies. However, by the provision of the polynucleotide and corresponding amino acid sequences of polypeptides and the peptide precursor of polytheonamides the present application for the first time provides also the possibility for the generation of novel molecules such as antibodies, antigen-binding fragments and similar antigen-binding molecules which are capable of specifically recognizing the polypeptides and the peptide precursor of polytheonamides. The antigen-binding fragment of the antibody can be a single chain Fv fragment, an F(ab′) fragment, an F(ab) fragment, and an F(ab′)₂ fragment, or any other antigen-binding fragment. Furthermore, due to the possibility to use other peptides as precursor of the peptide-based compound then the peptide precursor of polytheonamides of the amino acid sequence as defined in SEQ ID NO: 3, this other peptides may be used as well for the generation of above mentioned molecules capable of specifically recognizing said other peptides.

Therefore in a further embodiment of the present invention an antibody specifically recognizing a polypeptide or peptide precursor encoded by the nucleic acid molecule of the present invention as defined hereinabove or a peptide-based compound produced by the method of the present invention as defined hereinabove is provided.

Peptide based compounds generated by the methods of the present invention such as polytheonamides are cytotoxic as shown in Hamada et al., Tetrahedron Lett., 35 (1994), 719-720, Iwamoto et al, FEBS Lett., 584 (2010), 3995-3999 and assessed in Example 2 by similar methods as described in Iwamoto et al. FEBS Lett., 584 (2010), 3995-3999 (in particular by methods as described in Teta et al., Europ J of Chem Biol 11 (2010), 2506-2512; see Example 2 for details).

It is within the scope of the present invention to use the cytotoxic properties of peptide based compounds of the present invention, such as the members of the proteusins family, polytheonamides, by directing/targeting them to a selected cell population, thus eliminating or at least deplete this population and leaving other cell populations, different from the selected one, to the widest possible extent unaffected.

Cell populations and single cells which the peptide based compounds of the present invention may be targeted to, according to the methods of the present invention, are cell populations and single cells in an organism abnormal in presenting specific molecules, e.g., antigens, differing in kind and/or number from molecules presented on most other cell populations and single cells of the same organism, or on comparable cell populations and single cells in organisms of the same genus, which cell populations and single cells due of their statistical overrepresentation would be defined as normal in this respect. These abnormal cells and populations thereof are furthermore defined by the effect, which the cells have on the organism bearing said cells, which effect is detrimental for said organism in comparison to an organism not containing such cells or populations thereof. A non limiting example of such abnormal cells, to which the peptide based compounds of the present invention may be targeted to are single cells and cell populations growing in an uncontrolled manner, such as tumor cells. A further non limiting example of such abnormal cells are cells infected by viruses or by intracellular bacteria such as Chlamydia (C. trachomatis), Chlamydophila (C. pneumoniae and C. psittaci), Mycobacteria (M. tuberculosis or M. leprae), or Brucella (B. abortus, B. melitensis, B. canis, B. suis) or other parasites, e.g., the malaria causing parasite Plasmodium falciparum. It is a preferred object of the present invention to target such abnormal cells without affecting other, non-diseased cells, in affected individuals or in cell culture and to destruct the abnormal cells, e.g., tumor cells or diseased cells by contacting them with the peptide based compounds of the present invention. Referring to cells infected by viruses, intracellular bacteria or parasites, it is a preferred object of the present invention at least to release the intracellular bacteria or parasites by destructing the infected cells and making the infectious agents thereby more susceptible to the immune system or treatment by therapeutical means, such as antibiotics.

To achieve this, according to the present invention agents are generated comprising the peptide based compounds such as polytheonamides generated by the methods of the present invention which are linked to functional moieties targeting them to the diseased cells only (targeting moieties). After binding to the diseased cell, the cytotoxic peptide based compounds of the invention induce the destruction of the cell, e.g., polytheonamides by insertion into the membrane and generation of channels.

Moieties used for targeting of cytotoxic substances to diseased cells take advantage of the difference, either in kind and/or in number of specific molecules on the cell surface of the abnormal cells in comparison to the normal cells. A non limiting example of such differences in kind and/or in number of specific molecules on the cell surface of abnormal cells is the antigen expression on normal and on tumor cells. Therefore, according to the methods of the present invention, targeting moieties selectively targeting cells, because of the kind and/or number of specific molecules on their surface, are used in one embodiment of the present invention to direct the peptide based compounds to these cells and destroy these by this measure.

The term “targeting moiety” which will be defined in more detail hereinbelow includes but is not limited to receptors, antibodies, aptamers derivatives and fragments thereof capable of binding specifically to a target molecule or a target substance under physiological conditions. In binding to the target molecule the targeting moiety as used according to the methods of the invention delimits the cytotoxic effect of the peptide based compound of the present invention to the targeted cell. The target molecule or substance is a protein, peptide and derivatives thereof. The protein or peptide may be intracellular, extracellular or membrane-associated. Also included as target molecules are proteins, peptides and derivatives thereof produced by natural, recombinant or synthetic means. The target molecule that is bound by the aptamer of the present invention is not limited by size. The molecular weight of the target molecule may in general range from about 500 to about 300,000 daltons. Such proteins include but are not limited to toxins, enzymes, cell surface receptors, adhesion proteins, antibodies, cancer-associated gene products, hormones, cytokines and the like. The protein, peptide or derivative thereof is associated directly or indirectly with a disease in a mammal, including humans. The binding of the targeting moiety as used in methods of the present invention to the protein, peptide or derivative thereof prevents or inhibits the disease in the mammal by destroying the cell comprising said target molecule.

According to the methods of the present invention, the targeting moiety is linked covalently or non-covalently to the peptide based compound of the present invention. Linking of these targeting moieties may be achieved by a chemical conjugation (including covalent and non-covalent conjugations) see, e.g., international applications WO92/08495; WO91/14438; WO89/12624; U.S. Pat. No. 5,314,995; and European patent application EP 0 396 387.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” which has to be fused to a polypeptide or peptide-based compound according to the present invention may be a polypeptide derived from the same species or an aptamer, an antibody, or an antigen-binding fragment, variant, or analog thereof derived from an aptamer or an immunoglobulin polypeptide, or an immunoglobulin or non-immunoglobulin polypeptide of a different species. The term “heterologous expression” means expression of genes and their products in cells or organisms different from these said genes and their products originate from.

In this respect, in one embodiment the present invention also provides an agent comprising a peptide-based compound produced by a method as described hereinbefore which is covalently or non-covalently linked to a functional moiety.

Those skilled in the art will appreciate that conjugates may also be assembled using a variety of techniques depending on the selected compound to be conjugated. For example, conjugates with biotin are prepared, e.g., by reacting a polypeptide or peptide based compound of the present invention with an activated ester of biotin such as the biotin N-hydroxysuccinimide ester. Similarly, conjugates with a fluorescent marker may be prepared in the presence of a coupling agent, or by reaction with an isothiocyanate, preferably fluorescein-isothiocyanate. Conjugates of peptide based aptamers, antibodies, or antigen-binding fragments, variants or derivatives thereof are prepared in an analogous manner. Techniques for conjugating an antibody, or antigen-binding fragment, variant, or derivative thereof to various moieties, are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radio labeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62 (1982), 119-158.

Methods of conjugating DNA/RNA aptamers to peptides, polypeptides or proteins are known as well and might be performed according to the methods described in U.S. Pat. Nos. 6,623,926 B1, 7,910,297 B2, 7,270,950 B2, in the US application NO 2008/0058217 or as described in Cheng et al., Nucleic Acids Res., 11 (1983), 659-669; Niemeyer et al., Bioconjugate Chem., 12 (2001), 364-371; Takeda et al., Org. Biomol. Chem., 6 (2008), 2187-2194; Lovrinovic and Niemeyer, Biochem Biophys Res Commun. 335 (2005), 943-948; and in Robert and Szostak, Proc. Natl. Acad. Sci. USA 94 (1997), 12297-12302.

In certain embodiments, a moiety that enhances the stability or efficacy of a binding molecule may be conjugated. For example, in one embodiment, PEG can be conjugated to the binding molecules of the invention to increase their half-life in vivo. Leong et al., Cytokine 16 (2001), 106; Adv. in Drug Deliv. Rev. 54 (2002), 531; or Weir et al., Biochem. Soc. Transactions 30 (2002), 512.

Conjugates that are immunotoxins including conventional antibodies have been widely described in the art. The toxins may be coupled to the antibodies by conventional coupling techniques. The peptide based compounds of the present invention can be used in a corresponding way to obtain such immunotoxins. Illustrative of such immunotoxins are those described by Byers, Seminars Cell. Biol. 2 (1991), 59-70 and by Fanger, Immunol. Today 12 (1991), 51-54. In this respect whole monoclonal antibodies or fragments and derivates thereof may be used including single-chain Fv (ScFv), disulfide-stabilized Fv, bivalent disulfide-stabilized Fv and single-chain disulfide-stabilized Fv (SdsFv) (Wels et al., Cancer Immunol Immunother. 53 (2004), 217-226), wherein conjugates of the antibodies, or antigen-binding fragments, variants or derivatives thereof are prepared in an analogous manner.

Monoclonal antibodies may be prepared by any method known in the art such as the hybridoma technique (Koehler and Milstein, Nature, 1975, 256, 495-497), the trioma technique, the human B-cell hybridoma technique (Kozbor and Roder, Immunology Today, 4 (1983), 72-79) and the EBV-hybridoma technique (Cole et al., “Monoclonal Antibodies and Cancer Therapy”, pp. 77-96, Alan R. Liss, Inc., 1985).

Antibodies for use in the invention may also be generated using single lymphocyte antibody methods by cloning and expressing immunoglobulin variable region cDNAs generated from single lymphocytes selected for the production of specific antibodies by, for example, the methods described by Babcook et al., Proc. Natl. Acad. Sci. USA, 93 (1996), 7843-7848, WO 1992/02551, WO 2004/051268 and WO 2004/106377.

Humanized antibodies are antibody molecules from non-human species having one or more complementarity determining regions (CDRs) from the non-human species and a framework region from a human immunoglobulin molecule (see, for example, U.S. Pat. No. 5,585,089).

Chimeric antibodies are those antibodies encoded by immunoglobulin genes that have been genetically engineered so that the light and heavy chain genes are composed of immunoglobulin gene segments belonging to different species. By preserving the CDR-sequences and in general the binding specificity of a given antibody, other parts of the antibody, specifically most of the constant regions not responsible for the specificity are exchanged for corresponding regions of the organism into which said antibody has to be introduced, e.g., as a drug. For example, most therapeutical antibodies are generated first in the mice. Introduction of such antibodies into humans would induce an immune response. By reduction of the foreign sequence of the antibody chimeric antibodies are likely to be less antigenic.

The antibodies for use in the present invention can also be generated using various phage display methods known in the art and include those disclosed by Brinkmann et al., J. Immunol. Methods, 182 (1995), 41-50; Ames et al., J. Immunol. Methods, 184 (1995), 177-186; Kettleborough et al., Eur. J. Immunol., 24 (1994), 952-958; Persic et al., Gene, 187 (1997), 9-18; and Burton et al., Advances in Immunology, 57 (1994), 191-280; WO 90/02809; WO 91/10737; WO 92/01047; WO 92/18619; WO 93/11236; WO 95/15982; and WO 95/20401; and U.S. Pat. Nos. 5,698,426; 5,223,409; 5,403,484; 5,580,717; 5,427,908; 5,821,047; 5,571,698; 5,427,908; 5,516,637; 5,780,225; 5,658,727; 5,733,743; and 5,969,108. Techniques for the production of single chain antibodies, such as those described in U.S. Pat. No. 4,946,778, can also be adapted to produce single chain antibodies. Also, transgenic mice, or other organisms, including other mammals, may be used to express humanized antibodies.

The antibody fragments are also Fab′ fragments which possess a native or a modified hinge region. A number of modified hinge regions have already been described, for example, in U.S. Pat. No. 5,677,425, WO 99/15549 and WO 98/25971. Antibody fragments also include those described in WO 2005/003169, WO 2005/003170 and WO 2005/003171. Preferably, the antibody fragments envisaged for use in the present invention contain a single free thiol, preferably in the hinge region.

Antibodies which may be used according to the present invention include immunoglobulin molecules and immunologically active portions of immunoglobulin molecules, i.e. molecules that contain an antigen binding site that specifically binds an antigen. The immunoglobulin molecules of the invention can be of any class (e.g. IgG, IgE, IgM, IgD or IgA) or subclass of immunoglobulin molecule.

It is also within the scope of the present invention to use aptamers as targeting moieties in conjugates with the peptide based compounds of the present invention. The term “aptamer” as used herein is a single-stranded or double-stranded oligodeoxyribonucleotide, oligoribonucleotide, or a peptide or modified derivatives thereof that specifically bind a target molecule. The target molecule is defined as a protein, peptide and derivatives thereof. The aptamer is capable of binding the target molecule under physiological conditions. An aptamer effect is distinguished from an antisense effect as known in the art in respect of single- or double-stranded oligodesoxyribonucleotides or oligoribonucleotides, in that the aptameric effects are induced by binding to the protein, peptide and derivative thereof and are not induced by interaction or binding under physiological conditions with a nucleic acid. The aptamer contains at least one binding region capable of binding specifically to a target molecule or target substance.

Nucleic acid based aptamers are D-nucleic acids which are either single stranded or double stranded which specifically interact with a target molecule. The manufacture or selection of aptamers is, e.g., described in European patent EP 0 533 838 or in US application US 2010/0304991 A1. Basically the following steps are realized. First, a mixture of nucleic acids, i.e. potential aptamers, is provided whereby each nucleic acid typically comprises a segment of several, preferably at least eight subsequent randomized nucleotides. This mixture is subsequently contacted with the target molecule whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture having an increased ratio of nucleic acids specifically binding to the target from which the final binding nucleic acid is then optionally selected. These specifically binding nucleic acid(s) are referred to aptamers. It is obvious that at any stage of the method for the generation or identification of the aptamers samples of the mixture of individual nucleic acids may be taken to determine the sequence thereof using standard techniques. It is within the present invention that the aptamers may be stabilized such as, e.g., by introducing defined chemical groups which are known to the one skilled in the art of generating aptamers. Such modification may for example reside in the introduction of an amino group at the 2′-position of the sugar moiety of the nucleotides. Aptamers are currently used as therapeutical agents and it is also within the present invention that the thus selected or generated aptamers may be used as target moieties. The thus obtained small molecule may then be subject to further derivatization and modification to optimize its physical, chemical, biological and/or medical characteristics such as toxicity, specificity, biodegradability and bioavailability.

The generation or manufacture of spiegelmers which may be used or generated according to the present invention is based on a similar principle. The manufacture of spiegelmers is described in the international patent application WO 98/08856. Spiegelmers are L-nucleic acids, composed of L-nucleotides thus rather than aptamers which are composed of D-nucleotides as aptamers are. Spiegelmers are characterized by the fact that they have a very high stability in biological system and, comparable to aptamers, specifically interact with the target molecule against which they are directed to. In the purpose of generating spiegelmers, a heterogonous population of D-nucleic acids is created and this population is contacted with the optical antipode of the target molecule, in the present case for example with the D-enantiomer of the naturally occurring L-enantiomer of the CD3 kappa peptides. Subsequently, those D-nucleic acids are separated which do not interact with the optical antipode of the target molecule. However, those D-nucleic acids interacting with the optical antipode of the target molecule are separated, optionally determined and/or sequenced and subsequently the corresponding L-nucleic acids are synthesized based on the nucleic acid sequence information obtained from the D-nucleic acids. These L-nucleic acids which are identical in terms of sequence with the aforementioned D-nucleic acids interacting with the optical antipode of the target molecule will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

Therefore, in one preferred embodiment the present invention provides conjugates of the peptide based compounds of the present invention with targeting functional moieties, wherein the functional moiety comprises an antibody or an antigen-binding fragment thereof.

The functional/targeting moieties may target different molecules comprised by the target cells, however, in on particularly preferred embodiment the functional moiety conjugated to a peptide based compound of the present invention targets an antigen, wherein the antigen is a tumor antigen.

The present invention also provides a pack, kit or composition comprising one or more containers filled with one or more of the ingredients described herein. In one embodiment the present invention provides a composition comprising the nucleic acid molecule as defined hereinabove, the nucleic acid molecule which is capable of specifically hybridizing to said first nucleic acid molecule, the pair of nucleic acid molecules which correspond to the 5′ and reverse complement of the 3′ end of said first nucleic acid molecule mentioned above, the vector comprising the first or the second nucleic acid molecule, which second nucleic acid molecule is capable of specifically hybridizing to said first nucleic acid molecule, the host cell as defined hereinabove, a peptide-based compound produced by the methods of the present invention as defined hereinabove, the antibody specifically recognizing a polypeptide or peptide precursor encoded by said first nucleic acid molecule or a peptide-based compound produced by the methods of the present invention as defined hereinabove or the agent comprising a peptide-based compound produced by the method of the present invention which is covalently or non-covalently linked to a functional moiety as defined hereinabove.

Furthermore, in one embodiment the present invention provides a composition comprising at least one polypeptide which catalyzes at least one step of the biosynthesis of polytheonamides encoded by the nucleic acid molecule which was mentioned above as the first nucleic acid molecule or such a polypeptide produced by the method of the present invention; or a composition comprising the aforementioned ingredients, thus the first, second and the pair of nucleic acid molecules; the vector; the host cell; a peptide-based compound; the antibody or the agent of the present invention which is a kit or diagnostic composition.

Associated with such container(s), kit(s) or composition(s) can be a notice in the form prescribed by a governmental agency regulating the manufacture, use or sale of pharmaceuticals or biological products, which notice reflects approval by the agency of manufacture, use or sale for human administration. In addition or alternatively the kit or composition comprises reagents and/or instructions for use in appropriate screening assays or in appropriate therapeutic application. The composition or kit of the present invention is of course particularly suitable for the prevention and treatment of tumors. Furthermore, the composition or kit of the present invention is of course also suitable for the prevention and treatment of a disorder which is accompanied by the occurrence of cell populations with cell surface antigen compositions diverging from the antigen compositions of normal cells, e.g., as defined hereinabove, by cells infected by viruses, intracellular bacteria or intracellular parasites.

Therefore, in one embodiment the present invention provides a pharmaceutical composition comprising a peptide-based compound produced by the method of the present invention or the agent of the present invention as defined hereinabove; and optionally a pharmaceutically acceptable carrier.

As aforementioned, the present invention also provides pharmaceutical compositions. Such compositions comprise a therapeutically effective amount of a peptide based compound of the present invention such as a polytheonamide or an agent of the present invention comprising a peptide based compound which is covalent or non-covalently linked to a functional moiety, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol, sorbitol, trehelose and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient.

In a preferred embodiment the present invention provides a polytheonamide as a peptide-based compound obtainable by the methods of the present invention or the method for preparing a selected peptide-based compound or precursor thereof as defined hereinabove for use in the manufacture of a medicament for the treatment of a tumor.

In this context, the person skilled in the art will acknowledge that the present invention for the first time provides the necessary means and methods for producing the mentioned peptide based compound, in particular polytheonamides in an amount and purity sufficient and necessary for the preparation of a pharmaceutical composition. Therefore, while the prior art such as Hamada et al., Tetrahedron Lett, 35 (1994), 609-612; Hamada et al., Tetrahedron Lett, 35 (1994), 719-720; Hamada et al., J. Am. Chem., Soc 127 (2005), 110-118; supra, were able to extract polytheonamides from sponge in an amount rather sufficient for some biochemical analysis and cell based assays, the present invention now allows the formulation of a diagnostic composition at commercial scale and for the first time pharmaceutical compositions because the extraction of those peptide based compounds and polytheonamides in particular from sponges would not be sufficient to conduct the necessary pre-clinical and clinical trails for obtaining a marked approval and thus provision of a pharmaceutical composition.

### X. Techniques for Identification and Sequencing of Polypeptides

Detection, identification and characterisation of nucleotide sequences and of amino acids from one or more polypeptides, peptides and peptide-based compounds of the present invention may be attained by use of instruments such as mass spectrometers (see Example 8). Some examples which have been used in such tasks are the technique of desorption/ionisation of the analyte with the aid of an organic acid (matrix) through laser radiation (MALDI-TOF-MS) and the technique of ionisation by vaporisation of droplets of analyte solvated by a liquid mixture (spray) (ESI-MS) (Garden et al., J. Mass. Spectrom. 31 (1996), 1126-1130; De With et al., Peptides 18 (1997), 765-770; Garden et al., Proc. Natl. Acad. Sci. USA 95 (1998), 3972-3977; Redeker et al., Anal. Chem. 70 (1998), 1805-1811).

Preferentially, separation techniques, such as HPLC (High Performance Liquid Chromatography) or electrophoresis, are directly or indirectly coupled to the mass spectrometer.

The MALDI-TOF-MS technique is being much used in the analysis of macromolecules, especially peptides, proteins and nucleic acids. The possibility of investigating different classes of compounds is the result of the use of different and optimized combinations of matrixes and laser wavelengths. Various patent documents describe these applications in detail, of which: U.S. Pat. No. 6,235,478 and U.S. Pat. No. 6,277,573 which refer to the detection of DNA molecules with diagnosis purposes; U.S. Pat. No. 6,218,118 relates to a preparation of a mixture of compounds that allow the analysis of nucleotide sequences by mass spectrometry; U.S. Pat. No. 6,057,543 describes the improvement in spectrometer for the analysis of bio-molecules; U.S. Pat. No. 6,287,872 refers to support slides for the analysis of molecules with an elevated molecular weight; U.S. Pat. No. 6,265,716 deals with volatile matrixes for MALDI-TOF-MS spectrometry.

The document U.S. Pat. No. 6,278,794 describes the isolation and the computerized characterisation of proteins. In accordance with this method, the proteins are separated from a complex mixture by electrophoresis and, after isolating the bands, the sequencing is done using the MALDI-TOF-MS or ESI-MS technique.

The principles of chromatography, such as liquid chromatography, for example high-performance liquid chromatography and its more sensitive variants, nano-LC and capillary HPLC, are described in depth in several excellent textbooks including Scott, “Techniques and Practices of Chromatography”, Marcel Dekker 1995; Meyer, “Practical High-performance Liquid Chromatography”, 2d Ed., Wiley, New York, 1994; McMaster, “HPLC: A Practical User's Guide”, VCH Publishers, Inc., 1994; and Krustulovic and Brown, “Reversed-Phase HPLC: Theory, Practice and Biomedical Applications”, Wiley-Interscience, New York, 1982. Nano-LC is also described in a review article by Guetens et al. (Guetens et al., J. Chromatogr. B, 739 (2000) 139-150,). A discussion of coupled liquid chromatography and mass spectrometry is found in Niessen and van der Greef, “Liquid Chromatography-Mass Spectrometry”, Marcel Dekker, Inc., 1992.

Briefly, HPLC is a form of liquid chromatography, meaning the mobile phase is a liquid. The stationary phase used in HPLC is typically a solid, more typically a derivatized solid having groups that impart a hydrophilic or hydrophobic character to the solid. For example, silica gel is often used as the base solid and it is derivatized to alter its normally hydrophobic characteristics. Normal phase HPLC refers to using a non-polar mobile phase and a polar stationary phase. Reverse phase HPLC refers to a polar mobile phase and a non-polar stationary phase. Reverse phase HPLC is convenient because polar solvents such as water, methanol, and ethanol may be used and these solvents are easily and safely handled and disposed. The applicability of the above methods has been shown in the art in respect of polytheonamides (Hamada et al., J. Am. Chem. Soc. 127 (2005), 110-118). Their usability is further confirmed by selected further examples of marine-derived polypeptides which were purified using the methods described above and include: the neopetrosiamides (Williams et al., Org Lett. 7 (2005) 1473-4176), asteropine A (Takada et al. Chem. Biol. 13 (2006) 569-574), and the aculeines (Matsunaga et. al. ChemBioChem 12 (2011) 1-11

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The examples which follow further illustrate the invention, but should not be construed to limit the scope of the invention in any way. The following experiments in Examples 1 to 13 are illustrated and described with respect to the poy-gene cluster, the individual poy-genes as cloned, their products and new biotechnological methods using these products in the engineering of unique modified peptide based compounds; see also the Figures and the Tables 1 and 2 in this respect.

### Material and Methods

Detailed descriptions of conventional methods, such as those employed herein can be found in the cited literature; see also “The Merck Manual of Diagnosis and Therapy” Seventeenth Ed. edited by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art. For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation gene expression and protein purification referred to in this disclosure are available from commercial vendors such as BioRad, Stratagene, Invitrogen, QIAGEN, GE Healthcare, Merck, Novagen, Takava, New England Biolabs, GenScript. Synthesis of oligo/polynucleotides/genes and peptides may be performed by many suppliers/manufacturers such as GenScript, Invitrogen, Sigma-Aldrich, and ClonTech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

General recombinant protein expression methods in bacteria, expression optimization methods and following purification techniques are described in Terpe, Appl Microbiol Biotechnol 72 (2006), 211-222; Baneyx, Curr Opin Biotechnol 10 (1999), 411-421; Choi and Lee, Appl Microbiol Biotechnol 64 (2004), 625-635; Dummler et al., Microb Cell Fact (2005), 4:34; Dyson et al., BMC Biotechnol. (2004), 4:32; Georgiou and Segatori, Curr Opin Biotechnol 16 (2005), 538-545; Hengen, Trends in Biochemical Sciences 20 (1995), 285-286; Hochuli, et al., Nature Biotechnology 6 (1988), 1321-1325; Jana and Deb, Appl Microbiol Biotechnol 67 (2005), 289-298; Kane, Curr Opin Biotechnol 6 (1995), 494-500; Makrides, Microbiol Rev 60 (1996), 512-538; Marston, Biochem J 240 (1986), 1-12; Meinhardt, Appl Microbiol Biotechnol 30 (1989), 343-350; Pan and Malcolm, Biotechniques 29 (2000), 1234-1238; Shokri et al., Appl Microbiol Biotechnol 60 (2003), 654-664; Singh and Panda, J Biosci Bioeng 99 (2005), 303-310; Smith and Johnson, Gene 67 (1988), 31-40; Sørensen and Mortensen, J Biotechnol. 115 (2005), 113-128; Vallejo and Rinas, Microb Cell Fact (2004), 3:11; Walker et al., Biotechnol (NY) 12 (1994), 601-605; Zhang et al., Nat. Prod. Rep. 28 (2011), 125-151; Rodriguez et al., Methods in Enzymology 459 (2009), 339-365; Wenzel and Müller, 16 (2005), 594-606; Mus-Veteau, Methods and Protocols: Heterologous Expression of Membrane Proteins 601 (2010), 1^{st} Edition 1-272; Shi et al (2011) Journal of the American Chemical Society, 133(8), 2338-41.

### Example 1

### Heterologous Expression of the Polytheonamide (Poy) Gene Cluster

Heterologous expression was performed using the pETDuet™ (Merck KGaA, Darmstadt/Germany) vector suite in combination with various E. coli BL21 derivative strains (Merck KGaA, Darmstadt/Germany). Four transcripts were expressed through co-expression of pETDuet™-1 and pCDFDuet™-1-derived plasmids. Two opening reading frames in either pETDuet-1 or pCDFDuet-1 encoded an N-terminal, 6His-tag codon-optimized poyA and wild-type poyK. The other two opening reading frames of the complementary pETDuet-1 or pCDFDuet-1 plasmids contained poyJ and a transcript encoding poyB, poyC, poyA, poyD, poyE, poyF, poyG, poyH, and poyI. Plasmids were transformed by chemical transformation (heat shock) in accordance with the supplier's manual.

Individual or co-transformed plasmids were expressed for polytheonamide production in Bl21(DE3)AI (Invitrogen, Carlsbad/CA, USA; Cat. No.: C6070-03) and Bl21(DE3)Star™ pLysS (Invitrogen, Cat No.: C6020-03) E. coli strains. The bacteria were cultured in TB medium, each containing 100 μg/ml ampicillin/carbenicillin, 25 μg/ml streptomycin/spectionmycin, and. 25 μg/mL chloramphenicol (for Bl21(DE3)Star™ pLysS) were added as well. The cultures were grown at 37° C. in rotary shaking culture at 200 rpm until they reach mid-log phase (OD600 ˜1.5-2.0; 3 to 4 hours). Cultures were cooled to below 16° C. and then induced with 1 mM IPTG (Invitrogen, Catalog No. 15529-019; final concentration; isopropyl-b-D-thiogalactoside) and incubated at 16° C. for 24-120 hours prior to chemical extraction. Crude extracts were prepared using 500 mL bacterial cultures that were sonicated prior to two sequential 1:1 (v/v) diethyl ether extractions. Rotary evaporation of the diethyl ether yielded ˜5 mL of aqueous crude extract that was again extracted with two sequential 1:1 (v/v) diethyl ether extractions and evaporated to dryness. Extracts were resuspended in 50-100 μl of DMSO (dimethyl sulfoxide). Alternatively, the 500 mL cultures were spun down at 5000×g for 20 min and the cell pellet was separated from the supernatant. Independently, 2×300 mL cholorform was added to either fraction and was stirred at room temperature for 1 hour with the cell mass or was 2× extracted with the culture supernatant. The cell mass was filtered through paper and removed and then both samples were rotavapped to dryness. The resulting samples were resuspended in 50-100 μl of a 1:1 ratio of chloroform:methanol.

### Example 2

### Cytotoxicity and Cell Viability Assays

To confirm the generation of polytheonamides, their derivatives, variants or homologs by the methods of the present invention, their presence in cell extracts and in the outcome fractions of affinity purification, cytotoxicity assays using 5-10 μl of crude extract are performed with B104 rat Neuroblastoma (Interlab Cell Line Collection (ICLC), accession no. ICLC ATL99008), SH-SY5Y Human Neuroblastoma (European Collection of Cell Cultures (ECACC), catalogue no. 94030304), and HeLa cells (ECACC, catalogue no. 93021013) in cell cultures as described in Teta et al., Europ J of Chem Biol 11 (2010), 2506-2512 on page 2511, Experimental section, Cell viability assay, disclosure content of which is incorporated hereby by reference.

In addition to that, the cytotoxicity of the chemically extracted peptides or polypeptides are tested on HeLa cells as described in Ueoka et al., Toxicon, 53 (2009), 680-684 at page 681, in Section 2.5 of the Materials and Methods part of, in 96 well plates by the MTT-assay (3-(4,5-dimethyl-2-thiazolyl)-2,5-diphenyl-2H tetrazolium bromide), as described therein in detail in respect to P388 murine leukemia cells in Section 2. on the same page, disclosure content of which is incorporated hereby by reference.

In addition, cell viability assays such as the classic Trypan Blue dye exclusion staining method are employed. Cell culture medium is then removed from the culture and replaced with a 1:10 (v/v) mixture of 0.4% sterile-filtered Trypan Blue stain in Hanks Buffered Salt Solution (HBSS). After 2 minutes incubation at room temperature (RT), the staining solution is removed by aspiration and replaced with culture medium. By inspection in an inverted microscope the dead (blue) cells are counted.

Furthermore, a variety of mass spectrometric and chromatographic techniques is used to determine the presence of polytheonamides in the crude extracts as described in Example 5, below.

### Example 3

### Heterologous Expression of Individual Polytheonamide (Poy) Genes

Each gene encoded in the polytheonamide gene cluster was cloned into pET28b vector (Merck KGaA, Darmstadt/Germany) to produce the corresponding N-terminal 6His-tagged protein. Each polytheonamide gene was also cloned into pET29b (Merck KGaA, Darmstadt/Germany) to produce the corresponding C-terminal 6His-tagged protein or wild-type protein. Additionally, each wild-type sequence has been cloned into pETDuet™-1 and/or pCDFDuet™-1 plasmids. Individual genes were expressed in a variety of E. coli Bl21 derivative strains.

Heterologous expression of 1 or 2 plasmids was performed in Bl21(DE3)AI and Bl21(DE3)Star™ pLysS E. coli strains as described in Example 1. The cultures were induced with 1 mM IPTG and incubated at 16° C. for 24-48 hours prior to polytheonamide (poy-) Gene products extraction. Polypeptide extraction is performed as described in Example 5 below or by chemical extraction until resuspension of extracts in 50-100 μl of DMSO as described in Example 1, supra.

### Example 4

### Heterologous Expression of Multiple Polytheonamide (Poy) Genes

Co-expression constructs expressing one of more polytheonamide genes with an N-terminal 6His-tag poyA-construct were expressed using the pETDuet™-1 and pCDFDuet™-1 compatible plasmids. pETDuet™-1 constructs were cloned to include N-terminal 6His-tag poyA and each other polytheonamide gene. pCDFDuet™-1 constructs were constructed harboring 1 or 2 polytheonamide genes in all combinations.

Heterologous expression of 1 or 2 plasmids were performed in Bl21(DE3)AI and Bl21(DE3)Star™ pLysS E. coli strains as described in example 1. The cultures were induced with 1 mM IPTG and incubated at 16° C. for 14-48 hours prior to peptide extraction. Peptide extraction is performed as described in Example 5 below or by chemical extraction until resuspension of extracts in 50-100 μl of DMSO as described in Example 1, supra.

A variety of mass spectrometric and chromatographic techniques is used to determine the presence of modified PoyA peptides produced in the E. coli cultures—see also Example 5, below.

### Example 5

### Purification of Polypeptides and Peptides Fusions Comprising an N- or C-Terminal His-Tag

Co-expression constructs comprising individual, multiple or all of the polytheonamide poyB to poyK genes are co-expressed with an N-terminal 6His-tag gene construct encoding a peptide or polypeptide of interest using the pETDuet™-1 and pCDFDuet™-1 compatible plasmids. pCDFDuet™-1, pACYCDuet™-1 (Merck KGaA, Darmstadt/Germany) and/or pCOLADuet™-1 (Merck KGaA, Darmstadt/Germany) constructs are constructed harboring 1 or 2 polytheonamide genes each in all combinations. pETDuet™-1 constructs are cloned to include each other polytheonamide gene and an N-terminal 6His-tagged gene of interest different from poyA gene. Said gene of interest is a variant, fragment, derivative or homolog of the poyA gene or a gene of a sequence not related to poyA.

Individual or co-transformed plasmids are expressed for precursor peptide and polypeptide production in Bl21(DE3)AI (Invitrogen, Carlsbad/CA, USA; Cat. No.: C6070-03) and Bl21(DE3)Star™ pLysS (Invitrogen, Cat No.: C6020-03) E. coli strains. The bacteria are cultured in Terrific Broth (TB) medium (2×YT or LB are possible as well) each containing 50 μg/ml ampicillin/carbenicillin and 50 μg/ml streptomycin/spectionmycin. During prolonged incubations 34 μg/mL chloramphenicol are added as well. The cultures are grown at 37° C. in rotary shaking culture at 200 rpm until they reach mid-log phase (OD600 ˜0.4; 2 to 3 hours). Cultures are induced then with 1 mM IPTG (final concentration; isopropyl-b-D-thiogalactoside) and incubated at 16° C. for 24-48 hours prior to chemical extraction. Peptide extraction is performed as described in below or by chemical extraction until resuspension of extracts in 50-100 μl of DMSO as described in Example 1, supra. If no soluble proteins are obtained, modification of growth temperature, IPTG concentration (0.05-1 mM), addition of tRNA or chaperone genes is performed.

Peptide extraction is performed as described below or by chemical extraction until resuspension of extracts in 50-100 μl of DMSO as described in Example 1, supra.

Following cell lysis, purification is performed in accordance to the manual of the supplier (QIAGEN; Hilden/Germany) of Ni-NTA Protein agarose (QIAGEN Cat. No.: 30210, 30230 or 30250) which is used for the purification of peptides and polypeptides comprising either an N- or C-terminal His-tag. Purification under native conditions is preferred. Therefore, after treatment with lysozyme cells are lysed by sonication or homogenization. To prevent protein degradation, cells and protein solutions are kept at 0-4° C. at all times. Some steps require optimization in dependency of the precursor peptide expressed, as the addition of protease inhibitors is in this respect in some cases also necessary.

Non-native conditions are used as well, in case of formation of insoluble aggregates containing the expressed peptide or polypeptide of interest. Such inclusion bodies are solubilized by addition of denaturants such as 6 M GuHCl (Guanidine Hydrochloride) or 8 M urea again. Due to the non-native conditions the His-tag marked peptides and polypeptides bind efficiently to Ni-NTA Protein agarose and are renatured and refolded on the Ni-NTA column itself prior to elution (Holzinger et al. 1996), or in solution afterwards (Wingfield et al. 1995a).

### Lysis:

The bacterial culture is centrifuged (4.000×g, 5 min at 4° C.). The cell pellet is resuspended in lysis buffer (minimum volume 4 ml) with 10 mM imidazole at 2-5 ml per gram wet weight. During the lysis and in wash buffers imidazole is provided at low concentrations (10 mM; up to 20 mM for peptides/proteins exhibiting high binding affinities, 1-5 mM for peptides peptides/proteins which do not bind efficiently) to minimize nonspecific binding and reduce the amount of contaminants. 1 mg/ml Lysozyme and 3 Units per ml of original cell culture volume processed of Benzonase nuclease (Novagen/Merck KGaA, Darmstadt, Germany; Cat. No. 70664-3;) are added and the solution is incubated on ice for 30 min.

The lysate is centrifuged at 10,000×g for 20-30 min at 4° C. to pellet the cellular debris. Supernatant is stored on ice. Any insoluble material must be solubilized at this step using denaturing conditions before purification under denaturing conditions. 5 μl 2×SDS-PAGE sample buffer are added to 5 μl cleared lysate supernatant and stored at −20° C. for later SDS-PAGE analysis.

### Affinity Purification:

1 ml of Ni-NTA slurry (0.5 ml bed volume) is added to a 15 ml tube and briefly centrifuged. Supernatant is removed and 2 ml of lysis buffer are added. After gently mixing by inverting, the centrifugation step is repeated and the supernatant removed. 4 ml of this now cleared lysate is added the equilibrated matrix and mixed gently by shaking (200 rpm on a rotary shaker) at 4° C. for 60 min. The lysate-Ni-NTA mixture is loaded into a column and the column flow-through is collected and saved for SDS-PAGE analysis.

After two wash steps with 5 bed volume (2.5 ml) of wash buffer, the protein is eluted 4 times with 0.5 ml elution buffer. All two wash fractions and four eluate fractions are collected. An SDS-PAGE analysis is performed, wherein the samples saved from all steps of the purification are compared on the gel to monitor the purification success.

Separation from the Tag:

In case, the His-tag, GST-tag or another tag fused to the polypeptide of interest has to be separated from said polypeptide, additional steps are performed depending on the cleavage site and endopeptidase (Thrombin; GE Healthcare, Little Chalfont, United Kingdom; Cat. No.: 27-0846-01) or exopeptidase which has to be used (TAGZyme™-Kit; QIAGEN, Hilden/Germany, Cat. No.: 34300) before the step of elution of the polypeptide from the Ni-NTA or from Glutathione Sepharose, in case, the GST-tag was used (GE Healthcare, Little Chalfont, United Kingdom; Cat. No. 17-0756-01) in accordance to the manufacturer's recommendations.

In particular, when an N-terminal His-tag was used, the treatment is performed as described in (TAGZyme™ Handbook, QIAGEN, March 2003). In this, separation is performed as described in Protocol 2 at page 28 followed by Protocol 3 at page 29 for proteins with an intrinsic DAPase stop point due to the presence of the amino acids K, R, XXP, XP or Q at the N-terminus of the protein immediately after the His-tag (X represents any amino acid).

For proteins with an DAPase stop point due to the presence of the amino acid Q due to the introduction of this amino acid in front of the N-terminus of the protein immediately after last possible cleavage site by the enzyme DAPase, the experimental procedure is performed as described in Protocol 5 at page 33, followed by procedures of Protocol 6 at page 34 and Protocol 8 at page 37.

Temperature conditions of the DAPase reaction in the above-mentioned protocols are chosen from Table 4 at page 21 of the manual. Scale-up conditions for Protocol 6 are chosen from Table 17 at page 51 of the manual.

### Alternative Separation Method:

Alternatively the separation is performed as described in the respective protocols in Arneu et al., Methods in Molecular Biology, 2008, Volume 421, II, 229-243, in particular by the use of the methods described in the Methods section, in particular in section 3.2 at pages 237-238 in respect of removal of the His-tag by DAPase and Qcyclase treatment followed by the removal of DAPase and Qcyclase using IMAC, followed by removal of pyroglutamyl using DAPase as described in section 3.3 at pages 238-240, or as described in section 3.4 at page 240, when columns are used for the purification.

Initial lysis and purification are either performed as described in the above sections Lysis and Affinity Purification, or an IMAC purification of the His-tag is performed in front of the tag-removal by the method described in section 3.1 at page 237 of Arneu et al., Methods in Molecular Biology, 2008, Volume 421, II, 229-243, the disclosure content of which is hereby incorporated by reference.

Materials used in methods according to Arneu et al. and their preparation are performed as described in the Materials section 2.3 to 2.6 at page 235-236 therein.

### Characterization of the Produced Peptides, Polypeptides and Peptide Based Compounds:

The purified peptides, peptide based compounds and polypeptides are, if necessary, purified by further chromatographic steps, such as ion exchange, size exclusion or hydrophobic interaction chromatography and chemically characterized afterwards by MS (mass spectrometry) and NMR (nuclear magnetic resonance) methods. In respect of MS, methods such as MALDI-TOF (Matrix Assisted Laser Desorption/Ionization-Time Of Flight-Mass Spectrometry) are used to identify the prepropeptide using a mass range 10,000-20,000 Da), HPLC-ESI-HRMS (high performance liquid chromatography-electrospray ionization-high resolution mass spectrometry), ESI-FT-ICR MS (electrospray ionization-Fourier transform-ion cyclotron resonance mass spectrometry) and ESI-QqTOF-MS (electrospray-ionization-source quadrupole-quadrupole-time-of-flight-mass-spectrometry) using a mass range of 200-4,000 Da are used to detect multiply charged ions of the hydroxylated, dehydrated and methylated product peptides on the basis of the measured mass shifts, as compared to the unmodified peptides or proteins. The position of modified residues is determined by MS-MS (tandem mass-spectrometry) for sequencing peptides after tryptic or multienzymatic digestions. Tryptic digestions are carried out according to Rosenfeld et al., Anal. Biochem. 203 (1992) 173-179, the disclosure content of which is hereby incorporated by reference.

These tools allow detection of hydroxylated, dehydrated and methylated units on the basis of the measured mass shifts, as compared to the unmodified proteins. To determine the position of modified residues, peptides are sequenced by MS-MS after tryptic digestion. Since not all daughter ions might be detectable, a complementary approach consists of a complete hydrolysis of the peptides and HPLC(-MS) analysis of the released amino acids in comparison with the hydrolyzate of the unmodified fragments. In addition, feeding studies with [¹³C-Me]-methionine are conducted to gain further support for methylations on the basis of isotope mass shifts. (Methyl-)lanthionine bridges that might be formed by LanC-catalyzed cyclization are detected by MS-MS of the native peptides (interruption of the fragmentation pattern), by treatment with NiCl₂/NaBH₄ (reductive desulfuration and linearization) followed by MS-MS (Paz et al., Anal. Biochem. 36 (1970), 527-535, the disclosure content of which is hereby incorporated by reference) and by complete hydrolysis and detection of free (methyl-) lanthionine. (Li et al., Proc. Natl. Acad. Sci. U.S.A. 107 (2010), 10430-10435, the disclosure content of which is hereby incorporated by reference). Epimerizations are more difficult to detect, since the modification is not accompanied by mass shifts. Peptides arising from tryptic digests are therefore purified by HPLC and again subjected to total hydrolysis. The resulting amino acid mixtures are analysed either by HPLC-MS using a chiral column or after derivatization with chiral reagents, such as o-phthalaldehyde/isobutyryl-L-cysteine (OPA-IBLC; Bruckner et al. J. Chromatogr. 666 (1994), 259-273, the disclosure content of which is hereby incorporated by reference) or Marfey's reagent (Goodlett et al. J. Chromatogr. 707 (1995), 233-244, the disclosure content of which is hereby incorporated by reference) to identify the number of amino acids that have been epimerized. The identity of those amino acids that have been epimerized and/or modified is detected by degradation by mild acid hydrolysis as described for polytheonamides (Hamada et al., J. Am. Chem. Soc. 127 (2005), 110-118, in section “Stereochemistry of Amino Acids”, pages 112-114) or bogorols (Barsby et. al. J. Org. Chem. 71 (2006) 6031-6037; Goodlett et al., Journal of Chromatography A 707 (1995), 233-244))

### Example 6

### In-Vitro Treatment of Precursor Peptides

PoyA or other precursor peptides are generated by the methods of the present invention as described in Example 4. Individual polypeptides of the poy-cluster, catalyzing at least one of the steps of the biosynthesis of polytheonamides are generated as described in Example 3. Precursor peptide and the polypeptides are purified then by the methods of Example 5 or the purification, in particular of the precursor peptide is performed after the incubation described in the following. The precursor peptide of interest is incubated in-vitro with one or more of the polypeptides. In case of usage of the oxygen-sensitive enzymes of the radical SAM-family (poy B, C and/or D), the reaction is performed by use of a Glovebox, Coy chamber, or Argon- or other inert atmosphere, dithionite- and Fe²⁺-addition as described in Grove et al., Science 332 (2011), 604-607 and in detail in the Supporting Online Material, the disclosure content of which is hereby incorporated by reference.

### Example 7

### Expression of Selected Precursor Proteins from as-yet Uncharacterized Proteusin Pathways

Following bacterial strains are publicly available: Microscilla marina ATCC 23134, Desulfarculus baarsi DSM 2075, Chlorobium luteolum DSM 273, Nostoc punctiforme PCC 73102, Nostoc sp. PCC 7120 and Oscillatoria sp. PCC 6506. From these bacteria, precursor genes were amplified by PCR and cloned into expression vectors via suitable restriction sites. In addition, two precursor genes from Azospirillum sp. B510 and Pelotomaculum thermopropionicum SI are obtained by gene synthesis, since the strains are not publicly available. The ten precursors (see Table 1, SEQ ID NOs: 28, 30, 23, 34, 36, 38, 40, 42, 44, 43; and Table 3 showing the corresponding mature peptide fragments) were selected to cover a high structural diversity regarding peptide length, polarity, amino acid types and positions as well as bacterial taxonomy. For example, the M. marina peptide contains a large number of cationic residues in contrast to the lipophilic polytheonamide precursor. The P. thermopropionicum peptide contains a threonine residue after the conserved GG cleavage site, which in polytheonamides seems to be converted to the unique t-butylated unit.

TABLE 3
Selected precursor peptides from as-yet uncharact-
erized proteusin pathways. Only the sequence
following after the conserved nitrile hydratase
leader sequence and the GG-motif at its end
(LG or AG sometimes) is shown, the corresponding
sequence of the whole precursor peptides
may be found in Table 2, below.
The polytheonamide precursor is also
shown for comparison.

| Organism | Precursor region |
|---|---|
| | (SEQ ID No: 49) |
| M. marina | AGRRRRRRRRGPHIGRRRGGKGPRCRKRRFR |
| ATCC 23134 | |
| | (SEQ ID No: 50) |
| D. baarsi | ERLAAVI |
| DSM 2075 | |
| | (SEQ ID No: 51) |
| C. luteolum | EYVLCSGGWCQQE |
| DSM 273 | |
| | (SEQ ID No: 52) |
| N. puncti- | YMTTLASANASAKINPILPIRHSLVKTLR, |
| forme | |
| PCC 73102 | |
| | (SEQ ID No: 53) |
| | VNYSAVTVAIVKNTVKQNTNIITRAAVSVTALVTGA |
| | SIGASSVHL |
| | (SEQ ID No: 54) |
| Nostoc sp. | ALWTLTLLLIPIAHGALEEHNSRK |
| PCC 7120 | |
| | (SEQ ID No: 55) |
| Oscilla- | GCWIAGSRGCGFVTRT, |
| toria sp. | |
| PCC6506 | |
| | (SEQ ID No: 56) |
| | RRRGGSSRVITNTPGVPGCN |
| | (SEQ ID No: 57) |
| Azo- | VDIVTTITVTAIISAGVGGAAFSAVATVLAAGGIRG |
| spirillum | VCAKW |
| sp. B510 | |
| | (SEQ ID No: 58) |
| P. thermo- | TGCSDVYSFPICVPTYHDNTVPAPKAG |
| propionicum | |
| SI | |
| | (SEQ ID No: 48) |
| polytheon- | TGIGVVVAVVAGAVANTGAGVNQVAGGNINVVGNIN |
| amides | VNANVSVNMNQTT |

The ten precursors or variants, fragments (such as the cleaved forms as indicated in Table 3 above), derivatives or homologs thereof are expressed using vectors that allow purification via affinity chromatography by methods as described in Example 5. Initially, N-terminal tags, such as hexahistidine (vectors pET28a or pHis-8) and GST (pET41a), are used, since C-terminal tags might be modified by the polytheonamide enzymes. To optimize soluble proteins production, modification of growth temperature, IPTG concentration, addition of tRNA or chaperone genes are performed.

### Example 8

### Identification of Natural Products for Selected Proteusin Pathways

Since polytheonamides are the only known proteusins, it is envisaged to identify further members of this family from culturable bacteria. M. marina ATCC 23134 and the three cyanobacteria N. punctiforme PCC 73102, Nostoc sp. PCC 7120 and Oscillatoria sp. PCC 6506 are grown in larger cultures and proteins are extracted. The other publicly available strains (D. baarsi DSM 2075, C. luteolum DSM 273) are anaerobic symbionts and therefore difficult to cultivate. Extracts from the cultures above are analysed by ESI-LCMS to identify candidate molecular ions on the basis of the primary sequence of the propeptide (particularly the core peptide) and of the modification enzymes encoded in the gene clusters. The LCMS is conducted using a C18 reversed phase column and a mobile phase of 10% acetonitrile in water to 100% acetonitrile containing 0.1% formic acid. The post column detection is carried out with both UV (ultraviolet; wavelength=210-280 nm) and MS detection (mass range; m/z 500-4000 Da) using an ESI mass spectrometer. Compounds which contain accurate masses above 4000 Da are identified by their multiply charged ions. Peptides of interest identified by LC-MS using the conditions above are purified on preparative scale by either normal or reversed phase chromatography and HPLC. The molecular formula of the purified compounds is established by high-resolution mass spectrometry. The complete structures of the compounds are established by 1D and 2D NMR, IR, amino acid analysis of the hydrolysate and, if necessary, derivatization and/or degradation. These methods are used as described for other bacterial peptides in Miller et al., J. Org. Chem. 72 (2007) 323-330; Seyedsayamdost et. al., J. Am. Chem. Soc. 133 (2011) 11434-11437; Asolkar et al., J. Nat. Prod. 72 (2009) 396-402, which methods are incorporated herein by reference.

TABLE 1
Poy-gene cluster and sequences encoding the
individual genes therein

>Polytheonamide gene cluster (poy gene cluster)
(SEQ ID NO: 1)
CTATAAATCCGCACGGCTCAGGCCAAGTTGGGCGAGCATGGCATAGAGCG
TCCCCGTTTTCAATTCATCTCTGGGATTGCGCACAATGGTGAGCCGTTCG
CCATAGTACAAGGTCCCGTGGCTTCCCTTACCACGCTCGGGGACAAATTC
GACCCTGACGCCGCGTTGACGTCCAAGCCTTCTTAGTCGTCGAATGAATT
CGTTGCCAGTCATAGAAGGGAGTATCGGACATATATGACCGATGATCAAG
AGAAATGTGCAGCCTATTCCGATGGCCGTCATCAAATAGAGCGATTCGAA
TGCGAAGGGGCGTTAATGGGGTCGTATGAGTTTTTTGGCCACACAGGTGG
CAACTTTGCCGTGGAATCGATGGCAACTTTCGCGTGGAATATGCAGCAGT
GACTCCGAGTCTATCGTGGACGATAGAGGCGCTGTTTGTTAGGATAGGCC
ACTCGGCTCAAAAGTGGTGACAAAACCGGACGACACCCAGGTGAGCCACC
CATCTTTCAAGCCTCTACCCAAGAGGAGTTGTTGCAATGCTTCAGACCCA
ATGTTTGAAAGTACCAATCATCCCAGGCAAAGAGGCTGAGGTCAAAAATT
GGCTCGCCGCGCTTTCGACTCGACACCAGGAGGTGCTGGAAGCGATCACA
TCGGAAGACATCGCTGATGAGGCCATGTTTTACGCGAAAGAACCATCCGG
CGAGTTCCTCTACTTATACTCTCGTGCACCGGACTTGGCAGTAGCTGGCG
CCGCGTTTCAGAAATCCCAATTGCCCATCGATCAAGAGTTCAAGCGAATC
TCCGCGGAATGTCTGGACTACCGTTCAGCCATCCGACTTGAACTCCTTCT
TTCTGCGGATAGCCGCAACAAGTATGTGTATCCCTAAATCGGTCCACCTG
AGATTGGTACAGTGAGGTTCTCTATGCGACGTTGGATTTTCATGCTGATC
CACGTTTAAGGAGAACTCACCGTGCCTGCAGCTTCACTTGCACCGGCGCT
CAGGACGCGTGTTCGCTCAGCCAGGCTGCGACGCGCTGGAGGAACTCCGG
ATCGACGTGGCGGGTTTGATCGAAGTACTGGGCCATTGTCGATTCCCCAG
CTGTCGCCTTGAAGAGGTGATCGAGATTGGGGAAGAGCGCATACGTACAA
TCACCGCCCGCACTTTGAGCTGCGGTAACGAGCAGCTCGGCATCGCGCTC
GGCGGAGACTTGGAAGTCTTTGCTACCCTGACACACGAGTAGTGGGCACT
GGAGCCGGGTAATGAGTTCCACTGTCCGGTATAACAATAACTGTTTCAGC
CAAGTCGGGCTGCGGAACATCGCGAGGAGGTAGTGGGGGATATTGTCCGG
ATTCCACGGATGATCTGCCTTCACCAACTCTACCGCCTCCTGAACCTCCT
GAATCTGCTGCTCGATTTGCTCGTCCGTCAAGCCGATGTCTTTCCCCATG
TAGACAATCTGGTCGGTGATGACGTCCTCGATGCTCCGGCCCATGGTGGC
CATCAAGACCACTCCGCGCAGGGCAATACCCTCTTGCTCGGCGAGCACCA
GAGCCACAGTGCCGCCCTGGCTATGGCCAAGGAGGAAAACGGCACCTCCA
GCCGCTTCCGGGCGAGCGCGCAGAAATTCTAGGCATGCCCGGGCATCAGC
AATTTCCGAATCGAGGCCGCGAACCAGAGTGTCCTCGCCCATTTTCGTCG
TGCCCGCCCCGCGGGAATCGAAACGCAACCCCAGAAGGCCTTGCTCCGCC
AAAAAGTCCATAATTTCGTGCGTTCCCATATCTATTTCACCCGCGATCCC
GTGACGATCATGAGTGCCCGAACCACTCAGGAAGAGCACGGCTGGGAAGG
GGCCCGGACCTTCCGGAATGGAGAGCGTCGCACCGATTGGCGTCACCGGG
CCGGGGATAGTGACGTCCTCAAGACGGAAGCGGGTATTGTCGCGATAGTG
GTAGCGGAGCGGGACGGTGACCGGCTGATCCTCGTCAAGCCATTGCGGCA
CTGGCGGACCAGGGCGCTCCAGCCATCCGCGGACCCCTTGCGTCGGCACC
ATGCCTTCGATCATCAACCCGTGTTCATCGAGGTGAATCTCTTCAAGATG
CGAGGAGCGGTGCCACGTGCCGGACTCGGCGGCAAGGTCGAGTGCGGGAC
TCATCTGGTAGGGAACGGCCGCCAGCTGATTGGCGAGAAAGAGTCCAACT
GTCACCTCTTGGTCGAGGAGCTTGCGGCATGCCAAGGCGGCAAAGATCAG
TGCCTTGTGCCCCGGCACATTGTCGGCGGCGATGAACTGCGCCTCCCCGC
GCGGCACGACTTGCTCCGATCCATCGGGAAGCGTGGCCGTCACCTCGGCC
GATTCAAAGCGGACCTCCCAGCGAGTACCACCTGCCTCGCTGAGGTAGTG
CACGGGATCGAGTGCGCCGTTGCAGAGCAAGACGCTGCGTTGCCTCACAA
GTTCTCCCCCTAGACCCATCGCCACCGGATCAAGCTCAAGGAAGAATTCA
ATCTCGTGGAGATCACCCTCGCGTGGCCCCCGGTACACGGTCCACTGCGT
CGCATAGCGCTCATCGGCAATCGACAATGCATAGACGAGCGGCTCGCCGT
TGGGAAACAAAACGGCAACCCAGCCAGGAGCCACCTCTTGATCCGCAGAA
GCGTCCCCGGCGGGAGCGGCACTGGAGTCGGTACCGGCTTCAGTGGCGTC
CGCTGGCGTCGTACCCATTCTATTGGATTGGCTCATGGCTTACCTCCTCG
CCCGGAGATGGTGATTGATTAGCTGCCATTATCCTATCAACGCAATTATA
GGGTCGATGCACCAGCCTGTCCATCAGGGGCAATTCAAGCGGTTCCGTCG
CAAATTTTCATCTGAAGCCATTTGGGGTATGATCTCCCTCTATAATGAGC
GATGCATGAGGAGGGAGACAGACGATGGCGATCAGCTTCAAAGGTGCCCA
TTTTCCACCCAAAGTCATTTTGATGGGAGTCCGATGGTATCTGGCGTACC
CTTTGAGCACGCGCCACGTTGAGGAACTCATGGCAGAACGCGGGGTCCAC
GTCGATCATTCCACCGTCAACCGCTGGGTGGTGAAGTACAGTCCGCAGCT
CGAAGCCGAATTCCACCGGCGCAAACGTGCGGTGTGGACGAGTTGGCGGA
TGGATGAGACGTACATTAAAGTGAAAGGCGAGTGGAAATACCTCTACCGG
GCTGTTGACAAATTCGGCAAGACCATTGATTTTCTGCTCACGGAGCAGCG
TGATGAGAAAGCGGCCAGGAAGTTTCTCAACAAGGCGATTGGCCGCCACG
GCAGTGTGCCGGAGAAGATCACGATCGATGGTAGTGCGGCCAACGAAGCC
GCCATCAAGAGCTACAACAAGGATCACGGCATGTCAATCGAAATTCGCCA
GGTCAAGTATCTTAACAACATCGTGGAACAGGATCATCGAGGTGTGAAGC
GGATTACGCGGCCGATGCTGGGGTTCCAATCCTTTGACTCCGCTCAGTCC
ACTCTCACTGGCATCGAGCTGATGCACATGCTGCGAAAAGGGCAGCTTGA
AGATAGGGGTGAGCAGGGACTCAGTGTGGCCGATCAGTTCTACGCTCTGG
CCTCGTAGTCACCCTACCAAACGAGGTTCACTCACCCCAAAGCAGCGAGA
CACCCATATTTGCGACGGAACCTCACTACTTTCCAGCTCGGGTGAAAGAT
CCCAATTATTCTTAAAAAGTCGCTTGACAAAGTGCATGTGTTAGTTCAGA
ATTCCCCAATGTTCAAACTTTGACAGCATAAACTCGTAGTACCTTTCCCA
TCAGCGAACACAGCCGACGATAACTGATTCTGCTTGTTAGGAGATAGGAT
CATGAGTGATGTGCTTCTAGTCTCGGTACCGTACGCAGCCTTGCAGCACC
CATCCCCTGCATTGGGCTGTCTGCAGGCCGTGCTCCGGCGCCGTGGGATC
GAAGTCCACACCATGCATGCGAATCTCCGGTTCGCTGAACGGATCGGGAT
CGGGAACTATACATGGTTCGGTACCTACAGCCGGCCACAGTTGCTCGGCG
AGTTGACATTTGCCAAGGCTGCTTTTCCCGACTTCGAGCCCGATCTTCAC
GCCTATGCCCAGGTCATTAACGTACCGGAAGCGGAACATGCTATCCGCGA
CGTGCGGCAAGCCGCGGTTTCGTTCATCGACGAGATGGCTCAGCAGATCA
TCGAGCAAGATCCGAAGATTGTGGGATGTTCCTCAACGTTCCAGCAGAAT
TGCGCCTCGTTGGCGCTCTTGCGCCGGCTCCGCGAGCTGTCACCGGGCAT
CGTCACCGTCATGGGTGGAGCAAATTGCGAAAGCGAGATGGGGCGGGCTC
TCCATACGAACTTCGACTGGGTAGATTACGTCGTCTCGGGTGATGGGGAC
GAGGTGTTCCCCGACCTCTGCGAGCGGATTCTTCACAACGACATTGCGGG
CATCGCGTCTAACGAGGCGCTGCGCCGTTTCGTATTCACCCCCGCGTCCC
GACCGTTCGCTAATTTTCAGGTGGTCGAGCGCGCCACGACTCAGGACATG
GACGGATTACCCCTGCCGGACTACGACGATTACTTCCGCGAATTGGCAGA
GACAAAAATCGAATACTTTGCGACACCTGGGCTTCTCGTCGAGACCGCGC
GCGGTTGCTGGTGGGGGGAAAAACACCCGTGCACTTTTTGCGGCCTCAAC
GGTGGGTGCATGAGCTTTCGGGCGATGAGCCCGGAGAAGGCGGAGTGGCA
CATCTGCGAATTGTCCGCCCGCTACGGCATCGACGGGATCGAAGTGATTG
ATAATATCCTCGCGCCTTCTTATTTTAACACCGTTTTGCCGGCCCTCGCC
CGAAAGGAAAAGCGCTTGCGCCTAGCCTGCGAGGTAAAGGCCAACCTGAA
GCGGGAGCAAGTGAAAGCGCTGGCGGACGCGGGGGCAATCTGGGTGCAGC
CGGGCATCGAGGCCCTGCACGATGAAACGCTTAAGCTCATCGACAAGGGC
GCGACGGTCTGCCAGAACCTCCAGTTGCTGAAATGGGCTCGAGAATACGG
CGTCCATATTACCTGGAACTATCTACTTGGCATTCCGGGCGAACGGAGGG
CGTTATACAAGGAGGTCGCGGACCTCTTGCCCCTCATCATGCATTTACAG
GCACCTAATGGACCGGGCTCGCGGCTCAGCTTTGATCGGTTCAGCGTCTA
TCACACGCATGCAGACCATTATCAGCTCACGTTGCGTCCGGCATGGGGGT
ATTCGAACGTCTACCCATTGCCGATCAGCCAGCTCATAGATCTGGCCTAT
CACTTCGACGATGTCGGTCCTAACGCGGTGCTGCGCTCCGATTTTCCGGA
AATGGAACTCTTACGAGAGCGTTTGAAGGAATGGTGCGATCTGCAGCCGG
AGCCTTCCGGCTATGATGACCCGGCGGATTTGCCTCCCCTCCCCAGGCTA
GATGTCTTTGAACGAGACGACGGCAGACTCCTCATCGAAGACACGCGGCC
GTGCGCTCCGGCGACGCAGCGCGAGCTCTGTGGGCTCAGCGCCTATCTCT
ACACCGCGTGCGACCGAGGCCGTACTGCAGCACGGCTTCGGTCCGCTTGT
CACGACGAGGGCTTCTCTGAGGCCACTGCGGCCGACGTCGATCGCGAGTT
GACACGCCTCATTGACGACAAGCTCATGGCTTTTGTGAGCAACCGGTTTT
TGAGCCTCGCTGTGCGGGCGCCATACCCCGCCTGCCGACCCCTGAGTGAA
CACCCCGACGGGCAGGTGTATCTACGGCCCGTCCAGAAGCACCGCGAGCC
GCGCGAGCAGACGATTCAAGACGTATTCGGGCTTAAGCTCTGACTGGGCG
GGATGGGCTTCGGAAACTCTTGGAAAGGTATTGTATGCCAACAAACGATG
TGGTCCTGCTCAACATGCCGTATTCAGCCATCGAGCACCCGTCGATCTCC
CTCGGATACTTCTCGGCCAGCCTGAAGCAGCGCGGCATAAGTGTCGACAC
GATCTCCGCTAATGCGTTCTTCGCGCGTGATATCGGGCTCAAGGAGTACT
TTCTCTTCAGCAACTATTACAACAACGATTTGCTCGGCGAATGGACGTTC
TCGGGGGAGGCATTCCCGGATTTTCACCCCGATCACGACACCTATTTCCG
CGATCTTCAACTTCCGATTCCGGAGGCGAAGATCCGTCGCATTCGCGAGC
TCGCGGCAGGTTTCATCGACCAGATGACAGAGCGGGTCTTGTCTCAGAAC
CCACGCATCGTCGGCTGCAGCTCAACCTTTCAGCAAAACTGCGCCTCGCT
CGCCCTGCTGCGCCGGGTGCGGGAGCAAGCGCCGGAGGTCATTACAGTCA
TGGGCGGTGCGAACTGTGAGGGGCCAATGGGTAAGGCGCTCAAGCAGTGT
TTCGACTGGGTGGATCACGTGTTCTCAGGCGAGGCGGACGACCTTTTTCC
GGAGTTCTGTGCATTGATCCTGGACGCAACTGACCGGGAGCAAGCCATGC
GCCGCATCGCTGACTGGGCGCCTGGGTCGATCTTTCAGGTCTCAACAGGG
CTCATGCTGCAGGCGGAAGCGAGCGAGCGCTCTGTGGCTAAGGATCTCAG
TTCTCTCCCAACCCCCGATTACGACGACTACTTCCGCGACCTTCAGGAAG
CCGGGGTCGCCCGGCAGGTTCTGCCGGGGCTCATGCTTGAGACCTCGCGG
GGCTGCTGGTGGGGAGAGAAAGACGTCTGTACCTTCTGTGGGTTGAATGG
CGAATACATTAACTTCCGAGCCAAAGACCCCGACGTGGTGCACCGGGAGC
TCCGCGATCTCACGGCCCGCTACGGCATCAATGCGTTCGAAGTCGTAGAC
AATATCCTGTCTATGAAATACTTTAAAACGCTCTTGCCGCAGATTATCGA
ATCCGGAGAAAAGTATGGGTTCCTTTATGAAATCAAGGCGAACCTGAGAC
GCGATCACGTGGAGATGCTGGCCGCCGCTGGCGTGCTTTGGGTACAACCG
GGTATCGAGAGCCTGGATGATGGGGTACTCCGGCTGATTCACAAAGGTGC
CACGGCCTGCCAGAACTTGCAACTCCTCAAGTGGTGCCGCGAATACGGGC
TCTTTGTCATCTGGAACTACCTGTGCGATATCCCCGGTGAACATGACGAG
TGGCATGCCGACCTGGTCGACCTCCTGCCGCAAATCGTTCATTTTCAGCC
GCCTTCGTCGCCGGGATCACCCTTGCGTTTTGACCGCTTCAGCGTCTATC
ACAATTATCCGGATGAGCATGGCCTGGAGCTGACCCCCGCGTGGACCTAC
TCCTATATCTATCCGGTCAGCGACGCACACATCCAGCGGATCGCCTATTT
CTTCGACAATCACCGCCCAGATGCGGTCAACGATGGAAAGAACCGCCCGC
TCTGGGAGCAGGCGCGTGAGGAGCTCGTGGAGTGGCGCAATCTCCATTAC
CAGTATCAGGAAGATGACGTGTGGGCGGAGGTGGCGTCCGGCTCGCCGAT
GCTAAGCATGTCATATGGCGACGGGGATCTATTGATCCTTGAAGACACGC
GTCCCTGCGCCGTTCAATCGCGAATCGAGCTCCGAGGTGCCGCGGCCCGA
AATTTACGAGCTCTGCGACGAAGGTCGCAAGGCATCCACCGTGCTGTCGC
ACTGTCGGTCGTCCGGTTGTCCGGATCTCGACGCGGCTGAGGTCGACCGG
ATTCTCCAAACATTCCTAGATCACAAGATTATGGCCTTTGTCAGCGACCG
GTATTTGAGTCTTGCGGTGCGTGCGCCGTGGGCCTCGTACCCATCGATGG
AGTTGTTTCCAGGCGGGCGCGTTCTGCTCAAGCGCGCTGCGGCTCCGAAG
AAACCCCAAGAACTCACAGTCGCCGACGTGTTCGGGGTAAAGTCTAGATT
TCCATTCTAACCCAGAAAGGAGTCCACCATGGCAGACAGCGACAATACGC
CCACATCGAGGAAGGATTTTGAAACTGCGATCATTGCGAAGGCCTGGAAA
GATCCGGAATACTTGCGGCGCCTGCGGAGCAATCCGCGTGAAGTGCTGCA
GGAAGAACTTGAGGCCCTTCACCCTGGGGCGCAACTCCCCGACGATCTCG
GAATCTCGATCCACGAGGAAGATGAAAATCACGTCCATCTGGTCATGCCG
CGCCATCCGCAAAACGTGTCAGACCAGACCCTGACAGACGATGACCTCGA
TCAGGCCGCAGGTGGCACCGGAATCGGCGTTGTGGTGGCCGTGGTCGCCG
GTGCCGTCGCCAACACCGGCGCGGGTGTCAATCAGGTCGCAGGTGGCAAT
ATCAATGTGGTGGGGAACATCAACGTCAATGCCAACGTTAGCGTGAATAT
GAACCAGACCACATAATGCCTGCCCCGAAAGTACATCCCCCGCGGCGTTC
GGCTGGGGACGTATTGCTCGTGAAGAGGCTCTCAGGAGGCCAAGATGAAC
CTGCAGTCGATCGACTCTCAGGCAGTTCGTTCGAAGGTGGATGCGGAATA
CCCGTCGGCTGCGCATGTCAAGCGATTTCTGGAGGTTTGGTGCTCAGGGC
TCTATAAGAAAGAAGACTTGCTCACCCGACCGCAGGAAATCCTCGATGCT
CACCACGTAGCGATAGATCCGTCCCTCATCTCCGTTCTATTTGAATCGAA
ATTCTTGCGGGGGAAATCCGGCAAGTTTGACCTGCTACCGCCGCAGTTCG
ACGCGTTTCGCGACTTTATGATGACGAAGATCCAGTGGCGGCAACAAATC
CGCACTGGCTCTGCTCCTGCCGACCCCATATTTCGTGAATTTCGGGAGCG
TCAGATCCAGAGATGTGAGATCGAACTCGGCAGCGATCAGAACACCGCCA
TCGTTCACACGCCCGTCGTGTTCGAGCTGACGCGGGGCTGCTCGGTGAAG
TGTTGGTTTTGTGCGCTCGACGCACCGCCTCTCACGGGGATCTTTGACTA
TTCCCCGGAAAACGCACGATTTTTCCGGGACGTGCTTCGCGTCGTCAAAG
AACGTGATTGGTCCAGCGAGCAAATGGGCCAGCAGCTACTGGGGAACCGA
CCCGCTCGACAACCCCGATCAAGAGAAATTTAGCCTTGATTTCCGTGAAA
TCCTCGGCATGTATCCGCAGACGACGACGGCGATCCCACTGCGCGCGCCC
GAGCGCACGCGGAAGTTGCTTGAGGTGTCTCATGCCAGTGGCTGCCTCGT
CAACCGCTTCTCGGTTCGTACACCAGCGCAGCTCAGAAAGATTCACGATA
CGTTTTCGGCAGAGGAGCTGCTCTACACCGAGTTGGTCTTGCAGAATCTG
GAATCGGACAGTGTGAAGGCGCGTGCGGGCCGCTTGATCCATTTCGCCGA
TGATCTCCCCAAACTCGCTGCGAAAGATGAGGAAAAGCTGCTCAATATGC
TCCAAGAGCGAGAACCCGAGCTAGCCGCAAAAGCAACGTCGATTCTCATT
AATCTCCCAGGCTCCACCGAACCGATCATCAGGGCCACGAGTAACGCCGA
TGAAGAGGATACCTCCGAGGAATACAATGTGTCCATCAATGTCCCGGGCA
CTACGTCCTGCCTGACCGGCTTCAAGATCAACATGGTCGATCGCACTGTG
GAGCTGTTGAGCCCCTGTCCAGCAAACGAGCGATGGCCGCTCGGACACAT
TGTGTTTGAGGAAGGGACGTTCGACACAGCCGAAGACCTGAGGACGCTCA
TGCTCGGCATGATCTCTCGCAATATGGCTGAACGGGTCGTTCCCGAGTCG
TTGGTGCGCTTTGTCCCGCGGCTCATCTATCGCGAAGACCCGGGGGGTTC
CGCCTTGGTTCCGTGTTCGGCAACGGTGTGATCTGTCATGACCCGACTCG
ATCGGCGTACCTCCATCGTCTTGGCAACCTGCTCCGCGAGGGGAAAAAGC
GAGCCGGCGAGATCGCGATGCTGTGCTTTTATGAGTTCGGCGTACCTGAA
AATTACACGATGGGAAGCATCAACAACATGTTCCATCAGGGCATCATCGC
GGAGGAGCCGATCGCCACAGAGGCGCCGATCGCCGTGGCGGCTTACCAGT
AATGACGCCGAGGTCGCTTCCGACCGAGCCGCTCGCGGCCCCTGCCGCCG
CGGCTCTCGATGCCGCACTTCGACTGCTCAAGGAATACCTGGACGATATC
GGATATCACGGCATCTACAAATACCTGGCCACGGCGAACTATTATGCCGC
TTCGCCCTCCGTGTTCAATGCGTCTCGTCCCCAAAGCCTTGAGGCGTTCG
ATCGGCAAATCCACGAGGGACCGGATGACTGGATGCTCGCCAGATGCCTC
ACGACGTGCGCGCCGTGCCGTCTGGAAGCTCTGCCGCCCCCGGCGCGAAG
GGTGGCCGAGGTCCTGGCCGATGTCGGCCTCCTCGTGTGGAACGGGAACA
CGCTTGAGCAAGGAGGTTATCAGCTCATCTCTGTGTTCGATCGTTATATT
TTACTCGATGCGCGGATTCACTTCGGCGGCAGTCAGCTCCACGATGTCTA
TATTGGTCCCGACAGCCACCTCTTGCTGTATTACATGCCAGTGGAAGCGA
TCAGGCCGGCAGACCACATCCTCGACCTATGCACCGGCACCGGGGTGATC
GGACTTGGCCTGTCTCGATTCTCCGAGCATGTCGTCTCAACTGACATCGC
CCCGCCTGCCCTGCGTCTAGCGCATATGAATCGGGCACTCAACAACGCTG
AGGGGCGCGTGTCAATTCGCGCCGAGAATCTGCAAGAGACACTCGCTAGC
GATGAGTGTTTCGATCTGATCGCGTGCAATCCGCCATATGTCGCCGCACC
TCCCGAGCTTCCCACGCCGCTCTATGCGCAGGGTCCGGATCGGGACGGCC
TAGGCTATCTGCGCCTGCTGATGGAGCGGGCTCCTGAGAAGCTCAATCCG
GGTGGGCAGGCGATGTTTGTCGTCGATCTCATTGGCGACACGCATCGCCC
CTACTACTTCGACGACTTGGAGCGCATCGCGAAGGAGCAAGAGCTGTTTA
TCGAAGCCTTCATCGATAACCGTTTGAAGGCGGACGGGCAGCTCCCGGCT
TACAAGTTCCTCTACGCGCGGCTGTTTCATGGCACTCCCCCTGAAGAGAT
CGAACAGCGCATGAGGAACTTTATTTTCGATGAGCTGCACGCGTACTACT
ATTACATGACCACGCTCCGCGTGCGGCGCCGCAAACCATCCGGTTTGCGT
GTACTCGACCGGTACAGGATCACCAGCTATGATGAGTTCTTCCAGCAGTC
GTGAGGGAGACGCCCTCGCCCATGCTGCTGATCCCACAAGGTCAATTGCG
GACGACATCACTTGTGGGCTCGACTCGCTAGGCGGCCCTCCCACGGTATG
CGATGCTGAACATCCGGTCCCATTTGAGGAGCTATGGCTCGGGTTCGTCG
CGCACGCGGCTCGTGCGCTTGAACCGCTCATCGCGCCGTCGTTGCACGCG
GCTGTGTTACACGATCCGCACGGACCCCTGCGCTCGCTGCTCATCGAGCT
GGCCGAGATGGGCGCGCCAGCGGCTTTCGAGTTGTTTGCACTCTATCGCA
TCCGGGAGGCGAGTGCCGCGTCCGTCTTCGGAAGTGTCCGCGGGGTAGAG
AGTCGAGACACCTATAACGCATTCGTGAAGCACCTCGCCACTCAGCAGCT
CTCCCCGCTGTGGGATGCCTTTCCGGCCCTCAAGCCTCTGCTGGCCACCC
GTACCAATCTCTTTATAGCCGCCATGGCAGAGTTGTGCCAGCGGTGGGAG
GCCGATCACGTAGAGATCATGTCGGTCTTCCCGGAGTTACGAGGGATAGG
TGCACCGCAGCGCATTCGCCCCGGCTTATCCGATGCGCATGGCGGTGGGC
GAACCGTTACTCGCCTGTCATTCGCCGGTGGTGAGGCGCTTTTCTACAAG
CCACGCCCGGTCGACATGGAGTGGGGTTGTGCACAGTTTGTCGAGTGGTT
CAACGGCCAAGACCACGGGATGCCGCCGCTCCGGGCGCTTTCGGTCTTGC
CCAGAACCGACTATGGCTGGATGGAGGCGGCACGCCCCGCACTGTGCACA
CATGTTGACGCAGTCGCCCGATTCTACCACCGCGCAGGGATGCTGCTGGC
CCTCGCCGACCTTTTCTGCGGGGTCGATTTCCATAGTGAAAACCTGATTG
CAAGCGGTGAGTATCCGGTAATCGTGGATCTGGAGACCTTGTTTCATCCG
CTGGGGCCTTTCGAATCGCAGGGCGATGCCTTGGAGCGCACAGAGCTGCT
GCCACGGCCCATCTACTGTGAAGATGGTGCCCCCTATGTCATCTGCGGGT
TAGGTGTGGTTCCTGGAAAGGCGGTCATTGAGCTCCGGCGGCGCGGATGG
ATCAACATCAATTGCGATAACATGGCGTCCTGTGACGTGACCGTCCCTTG
GCCTGTCGGTGGCGCCGTGCCTCGCAACAAAGAGGGTGCGGCGCCGTCGA
TCGCGACATGGTCGGGCGAGATCGTCTCGGGGTTCTCTGCTATGCATGTG
TTCTTCCGCACACGCCGGAACGAGCTGTGGAGCGCCGACGGGCCTATCGT
GCAAGCGTTTGGCGGCGGGCGTTCGCGGTTTCTCCTACGCGCGACGCGGA
TCTATGTGGAGCTACTGCGCCGGGCCGTGCAGCCGCAAGCGCTCGCAGCT
CACGCGTCATCGAGCCACATCTTCGATCGTCTGGCGCGCACGGGCCGGGG
ATGGGATGAGATCCATCGCGTGGAGCGGCAGGCGCTCGATCGCATGGACG
TGCCTTATTTCACCATGGAAACCACCGCGCAGTTTGTTCGCGGCGAGGGC
CGTACCATCCATGCATTCTTCGCCACGTCCGGGCTTCACATGGCACGGCG
CCGGTCGGAGTTCATAGACGATGTCATTCTAAATGACAGGGCCGCGTCGA
TTCGCAATGTTCTCGAACAATCGAGTTACAGCGGGGAGGCGACCCTACCG
GACTCCAGAGTCCAGACCGACGCTGGCGCAGGAGCCTGAGCTATGGATAA
CGCGACACACCATTTCGAACAGAGCCATTTTGGCACCTTACACGAAGTGG
AGGCTATACGACGAAGTGAGTATTGAGCTTCTTGAGTACGCTACGGCCGG
CTTTAGGTGGGAGATCACTTACGAGTCGCCCGAAGCAGTCGAGGTGATTG
ATTCCGAATACGTGCCACCGGAAAGTGACGCCGCGGGTGCCGCCGGTCTC
CGACGCTTCCGGCTACGGCTGGTTCGCGAAGGACATGTGCGCCTGGCGCT
CCAAATGATTTGTCCCTTCCGGAAGGACGATCCACCTGCCGCATCGGGTA
CGATCGAGCTGCAGGTGCGGCCATAAAGCGGGCGCTGGTCCCTCATCATA
GAGTGATAGCCGAGTGCCCGCACGAACTAAGGAGACAGATTATGGCAATG
GATGATACGCACGTTAGGCAGCTCCAGTCGATATGCCAGACTCAGCATGC
GCGCTGGACGCCGGGAAGCAACAATATGACGAGCCTCGAACTTGAGGAAG
CCATACGCCATCTCGGGGGAGCGGTTGGGGACGATGACACGCCATTCGAG
GAGATGGAGGCGGTTGGACGTGATCTGCACCTGCATTCGATGGCGCTACG
CGTGCTCAGTCCCGGGTTCGGGGCGTGACACCGGCGGCGACAGCCCCCCC
TGTTGAGTTTGACTGGCGCTCGCACAACGGGCGCTCTTACGTCACCTCGG
TCAAGTTCCAGGGTGCGTGTGGCACGTGCACTTGCTTCAGCACGACTGCG
GCCGTGGAATCGGCCATTTGCATCGCCACCCAGACTTCGCCGCAGATCAT
TCAAGGCGTCGAGGTTCCGGCTCTGTCCGAGGCGCAGATGTTCTATTGCG
GGGCGGCGTCACAAGACCGCACTTGTGCCTCCGGATGGTTTCTCCCGGCG
GCGCTCGCTTACCTGCAGAACACTGGGGTCGCCCCGTACTCATATTTCCC
CTATGAATCGGGAGACCAGCCGTGCTTGATCCAACCGGGCTGGGAATCTG
TAGTGACAAAGATCATAGGCTCCACCAAGCTCACCGCCCCCGATGAGATT
AAGTCGTGGATCGCCACCAGAGGGCCAGTGGCGATCATGATGGTGGCCTA
TGAGGATCTCTTCACTTATAAGGAAGGGATTTACCACCCGGTGTCGACGA
ACAAGCTCGGAGTACACTCGGTGTGCGTTGTCGGTTACAGCGACAACAAA
TGAGGCGTGGCTCTGCAAGAATAGCTGGTCGACACAATGGGGAGAGGACG
GCTACTTCTGGATGGCCTACGGCGTATGTGGCATGGGATCGTCCGTACAC
GGGATCAACGGTCTGGCCCTGGTTGACGGAAAGCCGCTCTCGCCGCGTCG
AACCCGTCGCCTGACACGGCGTTGCAGGGGCCAATGAACGCAGAGCGGGT
GCTTAGGAGGGATACATGACTCAGAATCCCGGCTACAGTCTTCCCGTCGA
GAGGGTTAACGAGCTGTCGCGGGAACAGTTCCGCAAAGACTACCTCGCTC
ACTCCCGTCCGGTCGTCGTCACGGGCGGCGTCCGGGAGTGGCCCGCGCTG
AAGCGATGGGAGCTTGAGACCCTCACCGAGCGCCTGCAAGACCGTACAGT
GGAGATCGCCTCAACCGCCAAGGGTATCTTCTCTTACGATCTTGAATCCC
CCAGGGCTAAATACGAATACATGGCATTCTCGGACGCAGCAGCTCTCGTG
GCACAGGGTCAGAGGGATGCCCAGTACTACATCATGCAGCTCTCGATAGA
ACACTACTTCTCCGAGCTGAGAGACGATATTTTGCGGCTCGACTTGCTTT
CGGGAGAGGCGTGTTCGCCGCACTTCTGGCTCGGCGGGGCCGATCTCGTG
ACCCCTTTACACTGGGACAACTTGCACAATCTTTACGGGCAGGTGCGGGG
ACGAAAGCGTTTCACCCTGTTTGCGCCTGCGGAACATGACAATCTCTATC
CATACCCAGCTACCGCGCTGTACGGACACATGTCGTATGCAAACCCTGAG
GCAAGTGAACAGTGGCCAAAGCTGCGCGACGCGGAGCGGTTTGAATGCAT
TCTGGCCCCCGGCGATCTGTTATTTCTTCCAGCGTTTTGGTGGCATCACG
TCCGCTCGCTTGAGCTCGCGATCTCCGTGAACTTCTGGTGGGTTCCGGGT
CTCTCGGGGTGCTTCGCCCAGCCTTCCTTCGCACGCTGCGGATGGCCTAC
CGGCGTGAACGTCTGACGGGTCTTGGTGCCCCCGTGTCAACATTCCCAGG
AGGGCCGATCGGCGCAGCCCGTTCAGCTCTCCGCAACGGGCAAACGTCCT
TCGCAATGCTGTTCGCAGCCTCGGCCTTGGAGAAGACGATTCGCGCCCGA
TGCTATGCGGTCGGCATTGTGACTGGGAAGATGCTACGCCGCGCCCGATC
GAGGTGCTTGACGCTGAACTTGCAGCTTGCGGTGCCTACCCGCCCGATCT
CGACCGCGCGCGCCTTGGCTCGTGGACTCACGCCATCAACCGCGTAGTCG
ATGGCGACTCCGAGACCGCATTGAGTGTGGCGGAGGCGACAACCATCGTG
GACGAGATCAGGATGTTCGTCACCGATATGCACTGATCGATCATCCTCAG
CCGCAATGCCCCTGGGAGGGTTCCGTCGCAAATATGGGTGTCTCGCTGCT
TTGGGGTGAGTGAACCTCGTTTGGTAGGGTGACTACGAGGCCAGAGCGTA
GAACTGATCGGCCACACTGAGTCCCTGCTCACCCCTATCTTCAAGCTGCC
CTTTTCGCAGCATGTGCATCAGCTCGATGCCAGTGAGAGTGGACTGAGCG
GAGTCAAAGGATTGGAACCCCAGCATCGGCCGCGTAATCCGCTTCACACC
TCGATGATCCTGTTCCACGATGTTGTTAAGATACTTGACCTGGCGAATTT
CGATTGACATGCCGTGATCCTTGTTGTAGCTCTTGATGGCGGCTTCGTTG
GCCGCACTACCATCGATCGTGATCTTCTCCGGCACACTGCCGTGGCGGCC
AATCGCCTTGTTGAGAAACTTCCTGGCCGCTTTCTCATCACGCTGCTCCG
TGAGCAGAAAATCAATGGTCTTGCCGAATTTGTCAACAGCCCGGTAGAGG
TATTTCCACTCGCCTTTCACTTTAATGTACGTCTCATCCATCCGCCAACT
CGTCCACACCGCACGTTTGCGCCGGTGGAATTCGGCTTCGAGCTGCGGAC
TGTACTTCACCACCCAGCGGTTGACGGTGGAATGATCGACGTGGACCCCG
CGCTCTGCCATGAGTTCCTCAACGTGGCGCGTGCTCAAAGGGTACGCCAG
ATACCATCGGACTCCCATCAAAATGACTTCGGGTGGAAAATGGGCACCTT
TGAAGCTGATCGCCAT
>poyA (precursor peptide)
(SEQ ID NO: 2)
ATGGCAGACAGCGACAATACGCCCACATCGAGGAAGGATTTTGAAACTGC
GATCATTGCGAAGGCCTGGAAAGATCCGGAATACTTGCGGCGCCTGCGGA
GCAATCCGCGTGAAGTGCTGCAGGAAGAACTTGAGGCCCTTCACCCTGGG
GCGCAACTCCCCGACGATCTCGGAATCTCGATCCACGAGGAAGATGAAAA
TCACGTCCATCTGGTCATGCCGCGCCATCCGCAAAACGTGTCAGACCAGA
CCCTGACAGACGATGACCTCGATCAGGCCGCAGGTGGCACCGGAATCGGC
GTTGTGGTGGCCGTGGTCGCCGGTGCCGTCGCCAACACCGGCGCGGGTGT
CAATCAGGTCGCAGGTGGCAATATCAATGTGGTGGGGAACATCAACGTCA
ATGCCAACGTTAGCGTGAATATGAACCAGACCACATAA
>poyB (putative radical SAM methyltransferase)
(SEQ ID NO: 4)
ATGAGTGATGTGCTTCTAGTCTCGGTACCGTACGCAGCCTTGCAGCACCC
ATCCCCTGCATTGGGCTGTCTGCAGGCCGTGCTCCGGCGCCGTGGGATCG
AAGTCCACACCATGCATGCGAATCTCCGGTTCGCTGAACGGATCGGGATC
GGGAACTATACATGGTTCGGTACCTACAGCCGGCCACAGTTGCTCGGCGA
GTTGACATTTGCCAAGGCTGCTTTTCCCGACTTCGAGCCCGATCTTCACG
CCTATGCCCAGGTCATTAACGTACCGGAAGCGGAACATGCTATCCGCGAC
GTGCGGCAAGCCGCGGTTTCGTTCATCGACGAGATGGCTCAGCAGATCAT
CGAGCAAGATCCGAAGATTGTGGGATGTTCCTCAACGTTCCAGCAGAATT
GCGCCTCGTTGGCGCTCTTGCGCCGGCTCCGCGAGCTGTCACCGGGCATC
GTCACCGTCATGGGTGGAGCAAATTGCGAAAGCGAGATGGGGCGGGCTCT
CCATACGAACTTCGACTGGGTAGATTACGTCGTCTCGGGTGATGGGGACG
AGGTGTTCCCCGACCTCTGCGAGCGGATTCTTCACAACGACATTGCGGGC
ATCGCGTCTAACGAGGCGCTGCGCCGTTTCGTATTCACCCCCGCGTCCCG
ACCGTTCGCTAATTTTCAGGTGGTCGAGCGCGCCACGACTCAGGACATGG
ACGGATTACCCCTGCCGGACTACGACGATTACTTCCGCGAATTGGCAGAG
ACAAAAATCGAATACTTTGCGACACCTGGGCTTCTCGTCGAGACCGCGCG
CGGTTGCTGGTGGGGGGAAAAACACCCGTGCACTTTTTGCGGCCTCAACG
GTGGGTGCATGAGCTTTCGGGCGATGAGCCCGGAGAAGGCGGAGTGGCAC
ATCTGCGAATTGTCCGCCCGCTACGGCATCGACGGGATCGAAGTGATTGA
TAATATCCTCGCGCCTTCTTATTTTAACACCGTTTTGCCGGCCCTCGCCC
GAAAGGAAAAGCGCTTGCGCCTAGCCTGCGAGGTAAAGGCCAACCTGAAG
CGGGAGCAAGTGAAAGCGCTGGCGGACGCGGGGGCAATCTGGGTGCAGCC
GGGCATCGAGGCCCTGCACGATGAAACGCTTAAGCTCATCGACAAGGGCG
CGACGGTCTGCCAGAACCTCCAGTTGCTGAAATGGGCTCGAGAATACGGC
GTCCATATTACCTGGAACTATCTACTTGGCATTCCGGGCGAACGGAGGGC
GTTATACAAGGAGGTCGCGGACCTCTTGCCCCTCATCATGCATTTACAGG
CACCTAATGGACCGGGCTCGCGGCTCAGCTTTGATCGGTTCAGCGTCTAT
CACACGCATGCAGACCATTATCAGCTCACGTTGCGTCCGGCATGGGGGTA
TTCGAACGTCTACCCATTGCCGATCAGCCAGCTCATAGATCTGGCCTATC
ACTTCGACGATGTCGGTCCTAACGCGGTGCTGCGCTCCGATTTTCCGGAA
ATGGAACTCTTACGAGAGCGTTTGAAGGAATGGTGCGATCTGCAGCCGGA
GCCTTCCGGCTATGATGACCCGGCGGATTTGCCTCCCCTCCCCAGGCTAG
ATGTCTTTGAACGAGACGACGGCAGACTCCTCATCGAAGACACGCGGCCG
TGCGCTCCGGCGACGCAGCGCGAGCTCTGTGGGCTCAGCGCCTATCTCTA
CACCGCGTGCGACCGAGGCCGTACTGCAGCACGGCTTCGGTCCGCTTGTC
ACGACGAGGGCTTCTCTGAGGCCACTGCGGCCGACGTCGATCGCGAGTTG
ACACGCCTCATTGACGACAAGCTCATGGCTTTTGTGAGCAACCGGTTTTT
GAGCCTCGCTGTGCGGGCGCCATACCCCGCCTGCCGACCCCTGAGTGAAC
ACCCCGACGGGCAGGTGTATCTACGGCCCGTCCAGAAGCACCGCGAGCCG
CGCGAGCAGACGATTCAAGACGTATTCGGGCTTAAGCTCTGA
>poyC (putative radical SAM methyltransferase)
(SEQ ID NO: 6)
ATGCCAACAAACGATGTGGTCCTGCTCAACATGCCGTATTCAGCCATCGA
GCACCCGTCGATCTCCCTCGGATACTTCTCGGCCAGCCTGAAGCAGCGCG
GCATAAGTGTCGACACGATCTCCGCTAATGCGTTCTTCGCGCGTGATATC
GGGCTCAAGGAGTACTTTCTCTTCAGCAACTATTACAACAACGATTTGCT
CGGCGAATGGACGTTCTCGGGGGAGGCATTCCCGGATTTTCACCCCGATC
ACGACACCTATTTCCGCGATCTTCAACTTCCGATTCCGGAGGCGAAGATC
CGTCGCATTCGCGAGCTCGCGGCAGGTTTCATCGACCAGATGACAGAGCG
GGTCTTGTCTCAGAACCCACGCATCGTCGGCTGCAGCTCAACCTTTCAGC
AAAACTGCGCCTCGCTCGCCCTGCTGCGCCGGGTGCGGGAGCAAGCGCCG
GAGGTCATTACAGTCATGGGCGGTGCGAACTGTGAGGGGCCAATGGGTAA
GGCGCTCAAGCAGTGTTTCGACTGGGTGGATCACGTGTTCTCAGGCGAGG
CGGACGACCTTTTTCCGGAGTTCTGTGCATTGATCCTGGACGCAACTGAC
CGGGAGCAAGCCATGCGCCGCATCGCTGACTGGGCGCCTGGGTCGATCTT
TCAGGTCTCAACAGGGCTCATGCTGCAGGCGGAAGCGAGCGAGCGCTCTG
TGGCTAAGGATCTCAGTTCTCTCCCAACCCCCGATTACGACGACTACTTC
CGCGACCTTCAGGAAGCCGGGGTCGCCCGGCAGGTTCTGCCGGGGCTCAT
GCTTGAGACCTCGCGGGGCTGCTGGTGGGGAGAGAAAGACGTCTGTACCT
TCTGTGGGTTGAATGGCGAATACATTAACTTCCGAGCCAAAGACCCCGAC
GTGGTGCACCGGGAGCTCCGCGATCTCACGGCCCGCTACGGCATCAATGC
GGTTCGAAGTCGTAGACAATATCCTGTCTATGAAATACTTTAAAACGCTC
TTGCCGCAGATTATCGAATCCGGAGAAAAGTATGGGTTCCTTTATGAAAT
CCAAGGCGAACCTGAGACGCGATCACGTGGAGATGCTGGCCGCCGCTGGC
GTGCTTTGGGTACAACCGGGTATCGAGAGCCTGGATGATGGGGTACTCCG
GCTGATTCACAAAGGTGCCACGGCCTGCCAGAACTTGCAACTCCTCAAGT
GGTGCCGCGAATACGGGCTCTTTGTCATCTGGAACTACCTGTGCGATATC
CCCGGTGAACATGACGAGTGGCATGCCGACCTGGTCGACCTCCTCCGCAA
ATCGTTCATTTTCAGCCGCCTTCGTCGCCGGGATCACCCTTGCGTTTTGA
CCGCTTCAGCGTCTATCACAATTATCCGGATGAGCATGGCCTGGAGCTGA
CCCCCGCGTGGACCTACTCCTATATCTATCGGTCAGCGACGCACACATCC
AGCGGATCGCCTATTTCTTCGACAATCACCGCCCAGATGCGGTCAACGAT
GGAAAGAACCGCCCGCTCTGGGAGCAGGCGCGTGAGGAGCTCGTGGAGTG
GCGCAATCTCCATTACCAGTATCAGGAAGATGACGTGTGGGCGGAGGTGG
CGTCCGGCTCGCCGATGCTAAGCATGTCATATGGCGACGGGGATCTATTG
ATCCTTGAAGACACGCGTCCCTGCGCCGTTCAATCGCGAATCGAGCTCCG
AGGTGCCGCGGCCCGAATTTACGAGCTCTGCGACGAAGGTCGCAAGGCAT
CCACCGTGCTGTCGCACTGTCGGTCGTCCGGTTGTCCGGATCTCGACGCG
GCTGAGGTCGACCGGATTCTCCAAACATTCCTAGATCACAAGATTATGGC
CTTTGTCAGCGACCGGTATTTGAGTCTTGCGGTGCGTGCGCCGTGGGCCT
CGTACCCATCGATGGAGTTGTTTCCAGGCGGGCGCGTTCTGCTCAAGCGC
GCTGCGGCTCCGAAGAAACCCCAAGAACTCACAGTCGCCGACGTGTTCGG
GGTAAAAGTCTAG
>poyD (radical SAM-dependent)
(SEQ ID NO: 8)
ATGAACCTGCAGTCGATCGACTCTCAGGCAGTTCGTTCGAAGGTGGATGC
GGAATACCCGTCGGCTGCGCATGTCAAGCGATTTCTGGAGGTTTGGTGCT
CAGGGCTCTATAAGAAAGAAGACTTGCTCACCCGACCGCAGGAAATCCTC
GATGCTCACCACGTAGCGATAGATCCGTCCCTCATCTCCGTTCTATTTGA
ATCGAAATTCTTGCGGGGGAAATCCGGCAAGTTTGACCTGCTACCGCCGC
AGTTCGACGCGTTTCGCGACTTTATGATGACGAAGATCCAGTGGCGGCAA
CAAATCCGCACTGGCTCTGCTCCTGCCGACCCCATATTTCGTGAATTTCG
GGAGCGTCAGATCCAGAGATGTGAGATCGAACTCGGCAGCGATCAGAACA
CCGCCATCGTTCACACGCCCGTCGTGTTCGAGCTGACGCGGGGCTGCTCG
GTGAAGTGTTGGTTTTGTGCGCTCGACGCACCGCCTCTCACGGGGATCTT
TGACTATTCCCCGGAAAACGCACGATTTTTCCGGGACGTGCTTCGCGTCG
TCAAAGACGTGATTGGTCCAGCGAGCAAATGGGCCAGCAGCTACTGGGGA
ACCGACCCGCTCGACAACCCCGATCAAGAGAAATTTAGCCTTGATTTCCG
TGAAATCCTCGGCATGTATCCGCAGACGACGACGGCGATCCCACTGCGCG
CGCCCGAGCGCACGCGGAAGTTGCTTGAGGTGTCTCATGCCAGTGGCTGC
CTCGTCAACCGCTTCTCGGTTCGTACACCAGCGCAGCTCAGAAAGATTCA
CGATACGTTTTCGGCAGAGGAGCTGCTCTACACCGAGTTGGTCTTGCAGA
ATCTGGAATCGGACAGTGTGAAGGCGCGTGCGGGCCGCTTGATCCATTTC
GCCGATGATCTCCCCAAACTCGCTGCGAAAGATGAGGAAAAGCTGCTCAA
TATGCTCCAAGAGCGAGAACCCGAGCTAGCCGCAAAAGCAACGTCGATTC
TCATTAATCTCCCAGGCTCCACCGAACCGATCATCAGGGCCACGAGTAAC
GCCGATGAAGAGGATACCTCCGAGGAATACAATGTGTCCATCAATGTCCC
GGGCACTACGTCCTGCCTGACCGGCTTCAAGATCAACATGGTCGATCGCA
CTGTGGAGCTGTTGAGCCCCTGTCCAGCAAACGAGCGATGGCCGCTCGGA
CACATTGTGTTTGAGGAAGGGACGTTCGACACAGCCGAAGACCTGAGGAC
GCTCATGCTCGGCATGATCTCTCGCAATATGGCTGAACGGGTCGTTCCCG
AGTCGTTGGTGCGCTTTGTCCCGCGGCTCATCTATCGCGAAGACCCGGAG
GGGTTCCGCCTTGGTTCCGTGTTCGGCAACGGTGTGATCTGTCATGACCC
GACTCGATCGGCGTACCTCCATCGTCTTGGCAACCTGCTCCGCGAGGGGA
AAAAGCGAGCCGGCGAGATCGCGATGCTGTGCTTTTATGAGTTCGGCGTA
CCTGAAAATTACACGATGGGAAGCATCAACAACATGTTCCATCAGGGCAT
CATCGCGGAGGAGCCGATCGCCACAGAGGCGCCGATCGCCGTGGCGGCTT
ACCAGTAA
>poyE (SAM-dependent methyltransferase
(nucleophilic))
(SEQ ID NO: 10)
ATGACGCCGAGGTCGCTTCCGACCGAGCCGCTCGCGGCCCCTGCCGCCGC
GGCTCTCGATGCCGCACTTCGACTGCTCAAGGAATACCTGGACGATATCG
GATATCACGGCATCTACAAATACCTGGCCACGGCGAACTATTATGCCGCT
TCGCCCTCCGTGTTCAATGCGTCTCGTCCCCAAAGCCTTGAGGCGTTCGA
TCGGCAAATCCACGAGGGACCGGATGACTGGATGCTCGCCAGATGCCTCA
CGACGTGCGCGCCGTGCCGTCTGGAAGCTCTGCCGCCCCCGGCGCGAAGG
GTGGCCGAGGTCCTGGCCGATGTCGGCCTCCTCGTGTGGAACGGGAACAC
GCTTGAGCAAGGAGGTTATCAGCTCATCTCTGTGTTCGATCGTTATATTT
TACTCGATGCGCGGATTCACTTCGGCGGCAGTCAGCTCCACGATGTCTAT
ATTGGTCCCGACAGCCACCTCTTGCTGTATTACATGCCAGTGGAAGCGAT
CAGGCCGGCAGACCACATCCTCGACCTATGCACCGGCACCGGGGTGATCG
GACTTGGCCTGTCTCGATTCTCCGAGCATGTCGTCTCAACTGACATCGCC
CCGCCTGCCCTGCGTCTAGCGCATATGAATCGGGCACTCAACAACGCTGA
GGGGCGCGTGTCAATTCGCGCCGAGAATCTGCAAGAGACACTCGCTAGCG
ATGAGTGTTTCGATCTGATCGCGTGCAATCCGCCATATGTCGCCGCACCT
CCCGAGCTTCCCACGCCGCTCTATGCGCAGGGTCCGGATCGGGACGGCCT
AGGCTATCTGCGCCTGCTGATGGAGCGGGCTCCTGAGAAGCTCAATCCGG
GTGGGCAGGCGATGTTTGTCGTCGATCTCATTGGCGACACGCATCGCCCC
TACTACTTCGACGACTTGGAGCGCATCGCGAAGGAGCAAGAGCTGTTTAT
CGAAGCCTTCATCGATAACCGTTTGAAGGCGGACGGGCAGCTCCCGGCTT
ACAAGTTCCTCTACGCGCGGCTGTTTCATGGCACTCCCCCTGAAGAGATC
GAACAGCGCATGAGGAACTTTATTTTCGATGAGCTGCACGCGTACTACTA
TTACATGACCACGCTCCGCGTGCGGCGCCGCAAACCATCCGGTTTGCGTG
TACTCGACCGGTACAGGATCACCAGCTATGATGAGTTCTTCCAGCAGTCG
TGA
>poyF (LanM, N-terminal dehydratase domain)
(SEQ ID NO: 12)
ATGATGAGTTCTTCCAGCAGTCGTGAGGGAGACGCCCTCGCCCATGCTGC
TGATCCCACAAGGTCAATTGCGGACGACATCACTTGTGGGCTCGACTCGC
TAGGCGGCCCTCCCACGGTATGCGATGCTGAACATCCGGTCCCATTTGAG
GAGCTATGGCTCGGGTTCGTCGCGCACGCGGCTCGTGCGCTTGAACCGCT
CATCGCGCCGTCGTTGCACGCGGCTGTGTTACACGATCCGCACGGACCCC
TGCGCTCGCTGCTCATCGAGCTGGCCGAGATGGGCGCGCCAGCGGCTTTC
GAGTTGTTTGCACTCTATCGCATCCGGGAGGCGAGTGCCGCGTCCGTCTT
CGGAAGTGTCCGCGGGGTAGAGAGTCGAGACACCTATAACGCATTCGTGA
AGCACCTCGCCACTCAGCAGCTCTCCCCGCTGTGGGATGCCTTTCCGGCC
CTCAAGCCTCTGCTGGCCACCCGTACCAATCTCTTTATAGCCGCCATGGC
AGAGTTGTGCCAGCGGTGGGAGGCCGATCACGTAGAGATCATGTCGGTCT
TCCCGGAGTTACGAGGGATAGGTGCACCGCAGCGCATTCGCCCCGGCTTA
TCCGATGCGCATGGCGGTGGGCGAACCGTTACTCGCCTGTCATTCGCCGG
TGGTGAGGCGCTTTTCTACAAGCCACGCCCGGTCGACATGGAGTGGGGTT
GTGCACAGTTTGTCGAGTGGTTCAACGGCCAAGACCACGGGATGCCGCCG
CTCCGGGCGCTTTCGGTCTTGCCCAGAACCGACTATGGCTGGATGGAGGC
GGCACGCCCCGCACTGTGCACACATGTTGACGCAGTCGCCCGATTCTACC
ACCGCGCAGGGATGCTGCTGGCCCTCGCCGACCTTTTCTGCGGGGTCGAT
TTCCATAGTGAAAACCTGATTGCAAGCGGTGAGTATCCGGTAATCGTGGA
TCTGGAGACCTTGTTTCATCCGCTGGGGCCTTTCGAATCGCAGGGCGATG
CCTTGGAGCGCACAGAGCTGCTGCCACGGCCCATCTACTGTGAAGATGGT
GCCCCCTATGTCATCTGCGGGTTAGGTGTGGTTCCTGGAAAGGCGGTCAT
TGAGCTCCGGCGGCGCGGATGGATCAACATCAATTGCGATAACATGGCGT
CCTGTGACGTGACCGTCCCTTGGCCTGTCGGTGGCGCCGTGCCTCGCAAC
AAAGAGGGTGCGGCGCCGTCGATCGCGACATGGTCGGGCGAGATCGTCTC
GGGGTTCTCTGCTATGCATGTGTTCTTCCGCACACGCCGGAACGAGCTGT
GGAGCGCCGACGGGCCTATCGTGCAAGCGTTTGGCGGCGGGCGTTCGCGG
TTTCTCCTACGCGCGACGCGGATCTATGTGGAGCTACTGCGCCGGGCCGT
GCAGCCGCAAGCGCTCGCAGCTCACGCGTCATCGAGCCACATCTTCGATC
GTCTGGCGCGCACGGGCCGGGGATGGGATGAGATCCATCGCGTGGAGCGG
CAGGCGCTCGATCGCATGGACGTGCCTTATTTCACCATGGAAACCACCGC
GCAGTTTGTTCGCGGCGAGGGCCGTACCATCCATGCATTCTTCGCCACGT
CCGGGCTTCACATGGCACGGCGCCGGTCGGAGTTCATAGACGATGTCATT
CTAAATGACAGGGCCGCGTCGATTCGCAATGTTCTCGAACAATCGAGTTA
CAGCGGGGAGGCGACCCTACCGGACTCCAGAGTCCAGACCGACGCTGGCG
CAGGAGCCTGA
>poyG (chagasin-like peptidase inhibitor)
(SEQ ID NO: 14)
ATGGATAACGCGACACACCATTTCGAACAGAGCCATTTTGGCACCTTACA
CGAAGTGGGGCTATACGACGAAGTGAGTATTGAGCTTCTTGAGTACGCTA
CGGCCGGCTTTAGGTGGGAGATCACTTACGAGTCGCCCGAAGCAGTCGAG
GTGATTGATTCCGAATACGTGCCACCGGAAAGTGACGCCGCGGGTGCCGC
CGGTCTCCGACGCTTCCGGCTACGGCTGGTTCGCGAAGGACATGTGCGCC
TGGCGCTCCAAATGATTTGTCCCTTCCGGAAGGACGATCCACCTGCCGCA
TCGGGTACGATCGAGCTGCAGGTGCGGCCATAA
>poyH (C1 peptidase)
(SEQ ID NO: 16)
ATGGCAATGGATGATACGCACGTTAGGCAGCTCCAGTCGATATGCCAGAC
TCAGCATGCGCGCTGGACGCCGGGAAGCAACAATATGACGAGCCTCGAAC
TTGAGGAAGCCATACGCCATCTCGGGGGAGCGGTTGGGGACGATGACACG
CCATTCGAGGAGATGGAGGCGGTTGGACGTGATCTGCACCTGCATTCGAT
GGCGCTACGCAGTGCTCAGTCCCGGGTTCGGGGCGTGACACCGGCGGCGA
CAGCCCCCCCTGTTGAGTTTGACTGGCGCTCGCACAACGGGCGCTCTTAC
GTCACCTCGGTCAAGTTCCAGGGTGCGTGTGGCACGTGCACTTGCTTCAG
CACGACTGCGGCCGTGGAATCGGCCATTTGCATCGCCACCCAGACTTCGC
CGCAGATCATTCAAGGCGTCGAGGTTCCGGCTCTGTCCGAGGCGCAGATG
TTCTATTGCGGGGCGGCGTCACAAGACCGCACTTGTGCCTCCGGATGGTT
TCTCCCGGCGGCGCTCGCTTACCTGCAGAACACTGGGGTCGCCCCGTACT
CATATTTCCCCTATGAATCGGGAGACCAGCCGTGCTTGATCCAACCGGGC
TGGGAATCTGTAGTGACAAAGATCATAGGCTCCACCAAGCTCACCGCCCC
CGATGAGATTAAGTCGTGGATCGCCACCAGAGGGCCAGTGGCGATCATGA
TGGTGGCCTATGAGGATCTCTTCACTTATAAGGAAGGGATTTACCACCCG
GTGTCGACGAACAAGCTCGGAGTACACTCGGTGTGCGTTGTCGGTTACAG
CGACAACAAAGAGGCGTGGCTCTGCAAGAATAGCTGGTCGACACAATGGG
GAGAGGACGGCTACTTCTGGATGGCCTACGGCGTATGTGGCATGGGATCG
TCCGTACACGGGATCAACGGTCTGGCCCTGGTTGACGGAAAGCCGCTCTC
GCCGCGTCGACCCGTCGCCTGA
>poyI (Fe(II)/alpha-ketoglutarate-dependent
oxygenase)
(SEQ ID NO: 18)
ATGACTCAGAATCCCGGCTACAGTCTTCCCGTCGAGAGGGTTAACGAGCT
GTCGCGGGAACAGTTCCGCAAAGACTACCTCGCTCACTCCCGTCCGGTCG
TCGTCACGGGCGGCGTCCGGGAGTGGCCCGCGCTGAAGCGATGGGAGCTT
AGAGCCCTCACCGAGCGCCTGCAAGACCGTACAGTGGAGATCGCCTCAAC
CGCCAAGGGTATCTTCTCTTACGATCTTGAATCCCCCAGGGCTAAATACG
AATACATGGCATTCTCGGACGCAGCAGCTCTCGTGGCACAGGGTCAGAGG
GATGCCCAGTACTACATCATGCAGCTCTCGATAGAACACTACTTCTCCGA
GCTGAGAGACGATATTTTGCGGCTCGACTTGCTTTCGGGAGAGGCGTGTT
CGCCGCACTTCTGGCTCGGCGGGGCCGATCTCGTGACCCCTTTACACTGG
GACAACTTGCACAATCTTTACGGGCAGGTGCGGGGACGAAAGCGTTTCAC
CCTGTTTGCGCCTGCGGAACATGACAATCTCTATCCATACCCAGCTACCG
CGCTGTACGGACACATGTCGTATGCAAACCCTGAGGCAAGTGAACAGTGG
CCAAAGCTGCGCGACGCGGAGCGGTTTGAATGCATTCTGGCCCCCGGCGA
TCTGTTATTTCTTCCAGCGTTTTGGTGGCATCACGTCCGCTCGCTTGAGC
TCGCGATCTCCGTGAACTTCTGGTGGGTTCCGGGTCTCTCGGGGTGCTTC
GTCCCAGCCTTCCTTCGCACGCTGCGGATGGCCTACCGGCGTGAACGTCT
GACGGGTCTTGGTGCCCCCGTGTCAACATTCCCAGGAGGGCCGATCGGCG
CAGCCCGTTCAGCTCTCCGCAACGGGCAAACGTCCTTCGCAATGCTGTTC
GCAGCCTCGGCCTTGGAGAAGACGATTCGCGCCCGATGCTATGCGGTCGG
CATTGATGACTGGGAAGATGCTACGCCGCGCCCGATCGAGGTGCTTGACG
CTGAACTTGCAGCTTGCGGTGCCTACCCGCCCGATCTCGACCGCGCGCGC
CTTGGCTCGTGGACTCACGCCATCAACCGCGTAGTCGATGGCGACTCCGA
GACCGCATTGAGTGTGGCGGAGGCGACAACCATCGTGGACGAGATCAGGA
TGTTCGTCACCGATATGCACTGA
>poyJ (putative transporter/hydrolase)
(SEQ ID NO: 22)
ATGAGCCAATCCAATAGAATGGGTACGACGCCAGCGGACGCCACTGAAGC
CGGTACCGACTCCAGTGCCGCTCCCGCCGGGGACGCTTCTGCGGATCAAG
AGGTGGCTCCTGGCTGGGTTGCCGTTTTGTTTCCCAACGGCGAGCCGCTC
GTCTATGCATTGTCGATTGCCGATGAGCGCTATGCGACGCAGTGGACCGT
GTACCGGGGGCCACGCGAGGGTGATCTCCACGAGATTGAATTCTTCCTTG
AGCTTGATCCGGTGGCGATGGGTCTAGGGGGAGAACTTGTGAGGCAACGC
AGCGTCTTGCTCTGCAACGGCGCACTCGATCCCGTGCACTACCTCAGCGA
GGCAGGTGGTACTCGCTGGGAGGTCCGCTTTGAATCGGCCGAGGTGACGG
CCACGCTTCCCGATGGATCGGAGCAAGTCGTGCCGCGCGGGGAGGCGCAG
TTCATCGCCGCCGACAATGTGCCGGGGCACAAGGCACTGATCTTTGCCGC
CTTGGCATGCCGCAAGCTCCTCGACCAAGAGGTGACAGTTGGACTCTTTC
TCGCCAATCAGCTGGCGGCCGTTCCCTACCAGATGAGTCCCGCACTCGAC
CTTGCCGCCGAGTCCGGCACGTGGCACCGCTCCTCGCATCTTGAAGAGAT
TCACCTCGATGAACACGGGTTGATGATCGAAGGCATGGTGCCGACGCAAG
GGGTCCGCGGATGGCTGGAGCGCCCTGGTCCGCCAGTGCCGCAATGGCTT
GACGAGGATCAGCCGGTCACCGTCCCGCTCCGCTACCACTATCGCGACAA
TACCCGCTTCCGTCTTGAGGACGTCACTATCCCCGGCCCGGTGACGCCAA
TCGGTGCGACGCTCTCCATTCCGGAAGGTCCGGGCCCCTTCCCAGCCGTG
CTCTTCCTGAGTGGTTCGGGCACTCATGATCGTCACGGGATCGCGGGTGA
AATAGATATGGGAACGCACGAAATTATGGACTTTTTGGCGGAGCAAGGCC
TTCTGGGGTTGCGTTTCGATTCCCGCGGGGCGGGCACGACGAAAATGGGC
GAGGACACTCTGGTTCGCGGCCTCGATTCGGAAATTGCTGATGCCCGGGC
ATGCCTAGAATTTCTGCGCGCTCGCCCGGAAGCGGCTGGAGGTGCCGTTT
TCCTCCTTGGCCATAGCCAGGGCGGCACTGTGGCTCTGGTGCTCGCCGAG
CAAGAGGGTATTGCCCTGCGCGGAGTGGTCTTGATGGCCACCATGGGCCG
GAGCATCGAGGACGTCATCACCGACCAGATTGTCTACATGGGGAAAGACA
TCGGCTTGACGGACGAGCAAATCGAGCAGCAGATTCAGGAGGTTCAGGAG
GCGGTAGAGTTGGTGAAGGCAGATCATCCGTGGAATCCGGACAATATCCC
CCACTACCTCCTCGCGATGTTCCGCAGCCCGACTTGGCTGAAACAGTTAT
TGTTATACCGGACAGTGGAACTCATTACCCGGCTCCAGTGCCCACTACTC
GTGTGTCAGGGTAGCAAAGACTTCCAAGTCTCCGCCGAGCGCGATGCCGA
GCTGCTCGTTACCGCAGCTCAAAGTGCGGGCGGTGATTGTACGTATGCGC
TCTTCCCCAATCTCGATCACCTCTTCAAGGCGACAGCTGGGGAATCGACA
ATGGCCCAGTACTTCGATCAAACCCGCCACGTCGATCCGGAGTTCCTCCA
GCGCGTCGCAGCCTGGCTGAGCGAACACGCGTCCTGA
>poyK (unknown)
(SEQ ID NO: 20)
ATGCTTCAGACCCAATGTTTGAAAGTACCAATCATCCCAGGCAAAGAGGC
TGAGGTCAAAAATTGGCTCGCCGCGCTTTCGACTCGACACCAGGAGGTGC
TGGAAGCGATCACATCGGAAGACATCGCTGATGAGGCCATGTTTTACGCG
AAAGAACCATCCGGCGAGTTCCTCTACTTATACTCTCGTGCACCGGACTT
GGCAGTAGCTGGCGCCGCGTTTCAGAAATCCCAATTGCCCATCGATCAAG
AGTTCAAGCGAATCTCCGCGGAATGTCTGGACTACCGTTCAGCCATCCGA
CTTGAACTCCTTCTTTCTGCGGATAGCCGCAACAAGTATGTGTATCCCTA
A
>ORF-1 (integrase; outside of poy gene cluster,
just downstream of poyI)
(SEQ ID NO: 24)
ATGGCGATCAGCTTCAAAGGTGCCCATTTTCCACCCGAAGTCATTTTGAT
GGGAGTCCGATGGTATCTGGCGTACCCTTTGAGCACGCGCCACGTTGAGG
AACTCATGGCAGAGCGCGGGGTCCACGTCGATCATTCCACCGTCAACCGC
TGGGTGGTGAAGTACAGTCCGCAGCTCGAAGCCGAATTCCACCGGCGCAA
ACGTGCGGTGTGGACGAGTTGGCGGATGGATGAGACGTACATTAAAGTGA
AAGGCGAGTGGAAATACCTCTACCGGGCTGTTGACAAATTCGGCAAGACC
AATTGATTTTCTGCTCACGGAGCAGCGTGATGAGAAAGCGGCCAGGAAGT
GTTCTCAACAAGGCGATTGGCCGCCACGGCAGTGTGCCGGAGAAGATCAC
GATCGATGGTGTGCGGCCAACGAAGCCGCCATCAAGAGCTACAACAAGGA
TCACGGCATGTCAATCGAAATTCGCCAGTCAAGTATCTTAACAACATCGT
GGAACAGGATCATCGAGGTGTGAAGCGGATTACGCGGCCGATGCTGGGGT
TCCAATCCTTTGACTCCGCTCAGTCCACTCTCACTGGCATCGAGCTGATG
CACATGCTGCGAAAAGGGCAGCTTGAAGATAGGGGTGAGCAGGGACTCAG
TGTGGCCGATCAGTTCTACGCTCTGGCCTCGTAG
>ORF-2 (toxin-antitoxin system: toxin component;
outside of poy gene cluster, just upstream of
poyK)
(SEQ ID NO: 26)
ATGACTGGCAACGAATTCATTCGACGACTAAGAAGGCTTGGACGTCAACG
CGGCGTCAGGGTCGAATTTGTCCCCGAGCGTGGTAAGGGAAGCCACGGGA
CCTTGTACTATGGCGAACGGCTCACCATTGTGCGCAATCCCAGAGATGAA
TTGAAAACGGGGACGCTCTATGCCATGCTCGCCCAACTTGGCCTGAGCCG
TGCGGATTTATAG
>M. marina ATCC 23134 precursor peptide
(SEQ ID NO: 28)
ATGACCCAACAAGAGATATTAGACAGATTCGGTAGTTTGGAAAAGTTGAT
TACGGACACCAACTTCCGAAACGCCCTCAAAAAAGATCCTCGTAAAGCCC
TTGCACAAGAACTTTCTGGTGTGACCATTCCTGATAATGTAAGCCTCATC
GTGCATGAGAACACCACCAATGAAATGCACATTATTTTATTGCCTGATGC
TGAAGTTTCGGGAGAAGACATGCCCGATGATGACCCAATGGAAGTGGTGT
TAGACAAGGCAATGGCAGATAAAAGTTTCAAAGATTTGCTGATGATAGAC
CCCAAAGGTGTGTTGGCAAAAGAGCTTCCAGATTTTTATGTACCTGACGA
GTTTAAGGTATATTTTCACGAAAATACAGCGACCGAATGGCACTTGTTGA
TTCCATCGTTGGAAACTGAAGATGAAGATGGTGAGCTAAGTGAAGATGAG
CTTGAGGCAGTTGCCGGAGGAGCTGGACGAAGGCGCCGCCGCCGCCGCCG
TGGCCCCCATATTGGTCGTCGTCGTGGAGGCAAAGGTCCTCGTTGCCGTA
AGAGAAGATTCCGTTAA
>D. baarsi DSM 2075 precursor peptide
(SEQ ID NO: 30)
ATGTCCTCGGATAATATGGCGCATTCCAGCGCGTGGGCCAAGGTCGTGGC
CAAGGCCTGGGCCGACGAGTCTTACAAAAATAAGCTGCTCAGCGATCCGG
CGGCGGTGCTGCGCGCCGAGGGCTTGGCCATCCCCGAGGGCGTGCGCCTG
ACGGTGCTGGAAAACAGCGCCACCCAGATCCATCTGGTGCTGCCGGTCGC
GCCCAGTGACGCGGCCGACCTGGAAGACGCCGCCCTGGGCGAGCGCCTGG
CCGCCGTAATCTAG
>C. luteolum DSM 273 precursor peptide
(SEQ ID NO: 32)
ATGGCGTGCAATAGTGATAGATCATTGTTGTTATCTCAACCAAAGACCAA
GGATGTCCCCATGGAAGCAAACGAACAGCAGCAGGCACTGGGCAAGATCA
TAGCCAACGCCTGGGCGGATGAAGGTTTCAAGCAGCAGTTCATCGAAAAC
CCTGCAGAGATCCTGAGAGCAGAGGGTATCAGTGTGCCGGATGGAATGAT
GGTCAACGTGATGGAGAATACCCCGACCTGCATGCATATCGTCCTCCCGC
AATCCCCGGACATTGATCTGGATGGGGCTGCTCTGGACGCACTTGCCGGC
GGAGAGTATGTATTATGTAGTGGTGGTTGGTGTCAGCAGGAGTGA
>N. punctiforme PCC 73102 precursor peptide 1
(SEQ ID NO: 34)
ATGAGCGAACAAGAACAAGCGCAAACTCGCAAAAACATCGAAGCCCGGAT
TGTTGCCAAAGCCTGGAAAGATGAAGGGTATAAACAAGAATTGCTTACCA
ATCCCAAAGCTATAATCGAGCGGGAATTTGGAGTGGAATTCCCTGCTGAA
GTTAGCGTACAAGTCCTAGAAGAGAATTCCACTTCTTTGTATTTTGTACT
GCCAATTAGTCCAGTAGCGATCGCTCAAGAATTATCTGAAGAGCAACTAG
AAGCGATCGCTGGTGGTTATATGACAACTCTCGCATCTGCAAACGCATCC
GCAAAAATAAATCCCATTTTGCCCATACGACACTCACTTGTGAAAACACT
TAGATAA
>N. punctiforme PCC 73102 precursor peptide 2
(SEQ ID NO: 36)
ATGACTCAACAAGAACAAGCGCAAACACGCCAAGATATCGAAGCCCGCAT
CATCGCCAAAGCTTGGAAAGATGAAGCATACAAACAAGAGTTATTAACCA
ATCCCAAAGCTGTAATTGAGCGGGAATTTGGAGTGGAATTTCCTGCTGAT
GTTAACGTGCAAGTCCTTGAAGAGAATCCTACCTCTTTGCATTTTGTACT
ACCAATTAGTCCGGTAGCAATCGCCCAAGAATTATCTGAAGAGGAATTAC
TAGCGCTCGCTGCTGGTGTAAATTATTCGGCCGTGACCGTAGCGATTGTC
AAAAATACCGTTAAACAAAACACAAATATAATCACGAGAGCTGCTGTCTC
AGTAACAGCCTTAGTCACTGGAGCCTCTATAGGCGCTTCAAGCGTACATC
TCTAA
>Nostoc sp. PCC 7120 precursor peptide
(SEQ ID NO: 38)
ATGAGTGAGCAAACTAAAACTCGTAAAGATGTTGAAGCACAAATCATTGT
GCAAGCATGGAAAGATGAAGCTTACAGACAGGAATTACTGAACAATCCTA
AAAAAATAGTTGAACAAGAATTTGGTGTTCAATTACCAGAGGGAATAACA
GTTCACGTCATGGAAGAAAATGCTTCTAACCTCTATTTTGTAATTCCTGC
ACGCCCTAACTTAGAAGATGTAGAATTATCAGATGAGCAGCTAGAAGCCG
TTGCTGGTGGAGCGTTATGGACACTTACGCTCCTTTTAATTCCTATCGCT
CATGGCGCTCTTGAAGAACATAATTCTAGAAAATAA
>Oscillatoria sp. PCC 6506 precursor peptide 1
(SEQ ID NO: 40)
ATGTCTACTCGCAAAGAAGCCGAAGAACAACTCGCCATCAAAGCTCTTAA
AGATCCCAGCTTCCGCGAAAAACTCAAAGCCAATCCTAAAGCAGTGATTT
CTTCAGAGTTTAACACTCAAGTGCCAGACGATCTGACAATTGAAGTAGTA
GAAGAAACAGCTACTAAGATGTACTTAGTTCTACCTGCTCCTGAAGCTGT
TGAAGAAGAATTATCTGAAGAACAATTAGAAGCTGTCGCTGGTGGCGGTT
GCTGGATTGCTGGTAGCCGTGGCTGCGGTTTTGTAACTCGCACTTAA
>Oscillatoria sp. PCC 6506 precursor peptide 2
(SEQ ID NO: 42)
ATGACTTCATCAACATCTCAACCGGAACCAATGACTCGTGAAGAACTACA
AGCCAAACTGATTGCCAAAGCTTGGCAGGACGAGTCATTTAAGCAAGAAC
TACTCAGTAACCCCACAGCAGTCATTGCTAAGGAAATGGGTGTGGATAAT
ATCCCTGGAATCACCATCCAGATAGTAGAAGAAACCCCTACTACCTATTA
CCTAGTGTTGCCATCTAAACCAACGGATGACACCGAAGAACTTTCTGATG
CGGAATTGGAAGCTATCGCAGGTGGTCGTCGCAGGGGTGGGAGTAGCCGG
GTGATTACCAACACCCCAGGCGTGCCTGGTTGCAATTAG
>Azospirillum sp. B510 precursor peptide
(SEQ ID NO: 44)
ATGACAGACCAAACGCAGTCCGCCCCGATGACCCGCCGCGACCTTGAGGC
GAAGATCGTCGCCCGCGCCTGGTCGGACGACGACTTCAAGGCGAAGTTCC
TGGCCGACCCCAAGGCGATGTTCGAGGAGCATCTGGGCACCAAACTACCC
GCCTCGCTGGTGATGACGGCGCACGAGGAAACCGCCGACACGATCCACTT
CGTCATCCCGGCCAAGCCGCGGATCGACCTGGACGAGCTGTCGGACGAGG
ATCTGGAGAAGGTGGCCGGCGGCGTGGACATCGTGACGACGATCACCGTC
ACCGCGATCATCTCGGCCGGTGTGGGCGGTGCCGCCTTCTCGGCGGTGGC
GACCGTTCTCGCCGCCGGTGGAATCAGGGGGGTGTGTGCAAAATGGTAG
>P. thermopropionicum SI precursor peptide
(SEQ ID NO: 46)
ATGATCGAAAGCGAAAAGAAACCCGTGACCCGCAAAGAATTGAAGGAGCA
AATCATCAGGAAAGCGCAGGAAGACCGGGAATTTAAGAAAGCATTGGTCG
GGAATCCCAAAGGAGCCGTTGAACAATTGGGCGTCCAACTTCCCGAAGAC
GTTGAGGTCAAAGTCGTTGAGGAATCCGCAGAGGTGGTTTATCTGGTGCT
GCCGGTCAATCCCGGCGAGTTGACCGGTGAGCAGTTGGATAATGTAGCGG
GCGGGACCGGCTGTTCCGATGTATATTCCTTTCCTATCTGCGTTCCCACC
TACCATGACAACACGGTACCGGCACCAAAGGCAGGGTAG
>Polytheo-For1: degenerated oligonucleotide
sequence for peptide sequence GIGVVVA
(SEQ ID NO: 61)
GGNATHGGNGTNGTNGTNGC
>Polytheo-For2: degenerated oligonucleotide
sequence for peptide sequence GAGVNQT
(SEQ ID NO: 62)
GGNGCNGGNGTNAAYCARAC
>Polytheo-Rev: degenerated oligonucleotide
sequence for peptide sequence for peptide sequence
VNMNQTT
(SEQ ID NO: 63)
GTNGTYTGRTTCATRTTNAC
>PolytheoFor1-nested primer (alias PolytheoXForl)
(SEQ ID NO: 64)
CCGGTGCCGTCGCCAACAC
>PolytheoFor2:-nested primer (alias PolytheoXFor2)
(SEQ ID NO: 65)
GCGGGTGTCAATCAGGTCGCA
>PolytheoRev1:-nested primer (alias PolytheoXRevl)
(SEQ ID NO: 66)
GACGTTGATGTTCCCCACCACATTGA
>PolytheoRev2:-nested primer (alias PolytheoXRev2)
(SEQ ID NO: 67)
CGTTGGCATTGACGTTGATGTTCC
>pWEBfor
(SEQ ID NO: 71)
CGCCAGGGTTTTCCCAGTCA
>Up-PolyTheo-Rev1
(SEQ ID NO: 72)
AGGTGGTACTCGCTGGGAGGT
>Up-PolyTheo-Rev2
(SEQ ID NO: 73)
AGAACTTGTGAGGCAACGCAG
>PT-opt-F
(SEQ ID NO: 74)
CAAGACATATGGCAGACAGCGACAACACC
>PT-opt-R
(SEQ ID NO: 75)
AGCAAGCTTTTAGGTGGTCTGATTCATGTTCAC
>PT-opt-F3
(SEQ ID NO: 76)
CATGAATTCTATGGCAGACAGCGACAACAC
>SAM1-R
(SEQ ID NO: 77)
GATGTCGACTCAGAGCTTCAGCCCGAATA
>SAM2-F
(SEQ ID NO: 78)
GCAAGAATTAATGCCAACAAACGATGTGGTC
>SAM2-R
(SEQ ID NO: 79)
CCTCGAGCTAGACTTTTACCCCGAACACGT
>SAM3-F
(SEQ ID NO: 80)
CAAGACATATGAACCTGCAGTCGATTGACTCT
>SAM3-R
(SEQ ID NO: 81)
GCTCGAGTTACTGGTAAGCCGCCACG
>NmethT-F
(SEQ ID NO: 82)
CAAGACATATGACGCCGAGGTCGCTTC
>NmethT-R
(SEQ ID NO: 83)
GCTCGAGTCACGACTGCTGGAAGAACTC
>Lanth-F
(SEQ ID NO: 84)
CAAGACATATGATGAGTTCTTCCAGCAGTCGTG
>Lanth-R
(SEQ ID NO: 85)
TCTCGAGTCAGGCTCCTGCGCCA
>NMethT-opt-F
(SEQ ID NO: 86)
CAAGACATATGACGCCGCGTAGCCTG
>NMethT-opt-R
(SEQ ID NO: 87)
GGCAAGCTTTAAGATTGCTGGAAAAACTCATC
>PT-opt-half-R
(SEQ ID NO: 88)
AGCAAGCTTTTAGGCCACCTGATTGACG
>PT_OPT_R_ADD_1_9
(SEQ ID NO: 89)
ATTAAGCTTTTAGACACCAATACCCGTGCCACCCGCTGCTTGAT
>for E.coli expression codon optimized poyA
(SEQ ID NO: 90)
ATGGCAGACAGCGACAACACCCCGACGTCCCGCAAAGATTTCGAGACTGC
CATTATTGCGAAAGCATGGAAGGACCCGGAATACTTGCGCCGTCTGCGTA
GCAATCCGCGTGAGGTGCTGCAAGAAGAGTTGGAGGCTCTGCACCCAGGT
GCACAGCTGCCGGACGATCTGGGCATCTCTATCCACGAAGAGGACGAAAA
CCACGTTCATCTGGTCATGCCGCGTCATCCGCAGAATGTTAGCGATCAAA
CCCTGACGGATGATGACCTGGATCAAGCAGCGGGTGGCACGGGTATTGGT
GTCGTTGTGGCGGTGGTTGCGGGTGCCGTTGCTAACACCGGTGCGGGCGT
CAATCAGGTGGCCGGTGGCAACATCAATGTCGTGGGCAATATCAACGTTA
ACGCGAATGTCAGCGTGAACATGAATCAGACCACCTAA
>for E.coli expression codon optimized poyE
(SEQ ID NO: 92)
ATGACGCCGCGTAGCCTGCCGACCGAACCACTGGCCGCACCAGCAGCCGC
AGCATTGGACGCAGCTCTGCGCCTGCTGAAAGAATACCTGGACGATATCG
GTTACCACGGTATTTACAAATATCTGGCGACCGCTAACTATTATGCAGCT
AGCCCGAGCGTCTTTAATGCGAGCCGTCCGCAGTCCCTGGAGGCGTTCGA
TCGTCAAATCCACGAGGGTCCGGACGATTGGATGCTGGCGCGCTGTCTGA
CCACCTGCGCACCGTGTCGTCTGGAGGCCTTGCCGCCTCCGGCACGTCGT
GTTGCCGAGGTTCTGGCGGACGTTGGTCTGTTGGTCTGGAATGGTAATAC
CCTGGAACAGGGCGGCTACCAGCTGATTAGCGTGTTCGATCGCTACATTC
TGCTGGACGCCCGCATTCATTTTGGCGGTAGCCAATTGCACGACGTCTAT
ATCGGTCCGGATAGCCATCTGCTGCTGTATTACATGCCGGTTGAGGCGAT
CCGTCCGGCGGATCACATTCTGGACCTGTGTACGGGTACCGGCGTTATCG
GCTTGGGTTTGTCGCGCTTTAGCGAACATGTGGTCAGCACGGACATTGCG
CCACCGGCGCTGCGCCTGGCGCACATGAACCGTGCTCTGAACAATGCGGA
AGGCCGTGTGAGCATTCGTGCGGAGAATTTGCAGGAAACCCTGGCGTCCG
ATGAATGCTTTGACCTGATCGCGTGCAACCCGCCGTATGTGGCGGCACCG
CCGGAGCTGCCGACCCCGCTGTATGCCCAGGGCCCAGACCGTGATGGCCT
GGGTTACCTGCGTTTGCTGATGGAGCGTGCCCCGGAAAAACTGAACCCGG
GTGGTCAAGCGATGTTCGTGGTGGACCTGATTGGCGACACGCACCGCCCG
TACTATTTCGACGATCTGGAGCGTATTGCGAAGGAGCAAGAGCTGTTTAT
CGAGGCGTTCATCGATAACCGCCTGAAGGCTGATGGTCAACTGCCGGCAT
ATAAGTTCCTGTACGCACGTCTGTTTCATGGTACTCCTCCGGAAGAAATC
GAACAGCGTATGCGCAATTTCATTTTTGACGAGTTGCACGCCTACTACTA
TTACATGACCACGCTGCGTGTCCGTCGCCGTAAACCGAGCGGCTTGCGTG
TTCTGGATCGCTACCGCATCACGTCTTACGATGAGTTTTTCCAGCAATCT
TAA

TABLE 2
Polypeptides and precursor/peptide encoded by
the genes of the poy-cluster

>PoyA (precursor peptide); precursor peptide marked
in bold, marking includes GG-cleavage motif
(SEQ ID NO: 3) (= SEQ ID NO: 91)
MADSDNIPTSRKDFETAIIAKAWKDPEYLRRLRSNPREVLQEELEALHPG
AQLPDDLGISIHEEDENHVHLVMPRHPQNVSDQTLTDDDLDQAAGGTGIG
VVVAVVAGAVANTGAGVNQVAGGNINVVGNINVNANVSVNMNQTT
>PoyB (putative radical SAM methyltransferase)
(SEQ ID NO: 5)
MSDVLLVSVPYAALQHPSPALGCLQAVLRRRGIEVHIMHANLRFAERIGI
GNYTWFGTYSRPQLLGELTFAKAAFPDFEPDLHAYAQVINVPEAEHAIRD
VRQAAVSFIDEMAQQIIEQDPKIVGCSSTFQQNCASLALLRRLRELSPGI
VIVMGGANCESEMGRALHINFDWVDYVVSGDGDEVFPDLCERILHNDIAG
IASNEALRRFVFTPASRPFANFQVVERATTQDMDGLPLPDYDDYFRELAE
TKIEYFATPGLLVETARGCWWGEKHPCTFCGLNGGCMSFRAMSPEKAEWH
ICELSARYGIDGIEVIDNILAPSYFNIVLPALARKEKRLRLACEVKANLK
REQVKALADAGAIWVQPGIEALHDETLKLIDKGATVCQNLQLLKWAREYG
VHITWNYLLGIPGERRALYKEVADLLPLIMHLQAPNGPGSRLSFDRFSVY
HTHADHYQLTLRPAWGYSNVYPLPISQLIDLAYHFDDVGPNAVLRSDFPE
MELLRERLKEWCDLQPEPSGYDDPADLPPLPRLDVFERDDGRLLIEDIRP
CAPATQRELCGLSAYLYTACDRGRTAARLRSACHDEGFSEATAADVDREL
TRLIDDKLMAFVSNRFLSLAVRAPYPACRPLSEHPDGQVYLRPVQKHREP
REQTIQDVFGLKL
>PoyC (putative radical SAM methyltransferase)
(SEQ ID NO: 7)
MPINDVVLLNMPYSAIEHPSISLGYFSASLKQRGISVDTISANAFFARDI
GLKEYFLFSNYYNNDLLGEWIFSGEAFPDFHPDHDTYFRDLQLPIPEAKI
RRIRELAAGFIDQMTERVLSQNPRIVGCSSTFQQNCASLALLRRVREQAP
EVITVMGGANCEGPMGKALKQCFDWVDHVFSGEADDLFPEFCALILDATD
REQAMRRIADWAPGSIFQVSTGLMLQAEASERSVAKDLSSLPTPDYDDYF
RDLQEAGVARQVLPGLMLETSRGCWWGEKDVCIFCGLNGEYINFRAKDPD
VVHRELRDLTARYGINAFEVVDNILSMKYFKILLPQIIESGEKYGFLYEI
KANLRRDHVEMLAAAGVLWVQPGIESLDDGVLRLIHKGATACQNLQLLKW
CREYGLFVIWNYLCDIPGEHDEWHADLVDLLPQIVHFQPPSSPGSPLRFD
RFSVYHNYPDEHGLELTPAWTYSYIYPVSDAHIQRIAYFFDNHRPDAVND
GKNRPLWEQAREELVEWRNLHYQYQEDDVWAEVASGSPMLSMSYGDGDLL
ILEDIRPCAVQSRIELRGAAARIYELCDEGRKASTVLSHCRSSGCPDLDA
AEVDRILQTFLDHKIMAFVSDRYLSLAVRAPWASYPSMELFPGGRVLLKR
AAAPKKPQELTVADVFGVKV
>PoyD (radical SAM-dependent enzyme)
(SEQ ID NO: 9)
MNLQSIDSQAVRSKVDAEYPSAAHVKRFLEVWCSGLYKKEDLLTRPQEIL
DAHHVAIDPSLISVLFESKFLRGKSGKFDLLPPQFDAFRDFMMTKIQWRQ
QIRTGSAPADPIFREFRERQIQRCEIELGSDQNTAIVHIPVVFELTRGCS
VKCWFCALDAPPLIGIFDYSPENARFFRDVLRVVKDVIGPASKWASSYWG
IDPLDNPDQEKFSLDFREILGMYPQTTTAIPLRAPERTRKLLEVSHASGC
LVNRFSVRTPAQLRKIHDIFSAEELLYTELVLQNLESDSVKARAGRLIHF
ADDLPKLAAKDEEKLLNMLQEREPELAAKATSILINLPGSTEPIIRATSN
ADEEDISEEYNVSINVPGITSCLIGFKINMVDRIVELLSPCPANERWPLG
HIVFEEGTFDTAEDLRILMLGMISRNMAERVVPESLVRFVPRLIYREDPE
GFRLGSVFGNGVICHDPIRSAYLHRLGNLLREGKKRAGEIAMLCFYEFGV
PENYTMGSINNMFHQGIIAEEPIATEAPIAVAAYQ
>PoyE (SAM-dependent methyltransferase
(nucleophilic))
(SEQ ID NO: 11) (= SEQ ID NO: 93)
MTPRSLPTEPLAAPAAAALDAALRLLKEYLDDIGYHGIYKYLATANYYAA
SPSVFNASRPQSLEAFDRQIHEGPDDWMLARCLITCAPCRLEALPPPARR
VAEVLADVGLLVWNGNTLEQGGYQLISVFDRYILLDARIHFGGSQLHDVY
IGPDSHLLLYYMPVEAIRPADHILDLCIGTGVIGLGLSRFSEHVVSTDIA
PPALRLAHMNRALNNAEGRVSIRAENLQETLASDECFDLIACNPPYVAAP
PELPTPLYAQGPDRDGLGYLRLLMERAPEKLNPGGQAMFVVDLIGDTHRP
YYFDDLERIAKEQELFIEAFIDNRLKADGQLPAYKFLYARLFHGTPPEEI
EQRMRNFIFDELHAYYYYMTTLRVRRRKPSGLRVLDRYRITSYDEFFQQS
>PoyF (LanM, N-terminal dehydratase domain)
(SEQ ID NO: 13)
MMSSSSSREGDALAHAADPIRSIADDITCGLDSLGGPPTVCDAEHPVPFE
ELWLGFVAHAARALEPLIAPSLHAAVLHDPHGPLRSLLIELAEMGAPAAF
ELFALYRIREASAASVFGSVRGVESRDTYNAFVKHLATQQLSPLWDAFPA
GLKPLLATRINLFIAAMAELCQRWEADHVEIMSVFPELRGIGAPQRIRPG
LSDAHGGRTVTRLSFAGGEALFYKPRPVDMEWGCAQFVEWFNGQDHGMPP
LRALSVLPRTDYGWMEAARPALCTHVDAVARFYHRAGMLLALADLFCGVD
FHSENLIASGEYPVIVDLETLFHPLGPFESQGDALERTELLPRPIYCEDG
SAPYVICGLGVVPGKAVIELRRRGWININCDNMASCDVIVPWPVGGAVPR
NKEGAAPSIATWSGEIVSGFSAMHVFFRIRRNELWSADGPIVQAFGGGRS
RFLLRATRIYVELLRRAVQPQALAAHASSHIFDRLARTGRGWDEIHRVER
QALDRMDVPYFTMETTAQFVRGEGRTIHAFFATSGLHMARRRSEFIDDVI
LNDRAASIRNVLEQSSYSGEATLPDSRVQTDAGAGA
>PoyG (chagasin-like peptidase inhibitor)
(SEQ ID NO: 15)
MDNATHHFEQSHFGILHEVGLYDEVSIELLEYATAGFRWEITYESPEAVE
VIDSEYVPPESDAAGAAGLRRFRLRLVREGHVRLALQMICPFRKDDPPAA
SGTIELQVRP
>PoyH (C1 peptidase)
(SEQ ID NO: 17)
MAMDDTHVRQLQSICQTQHARWTPGSNNMISLELEEAIRHLGGAVGDDDT
PFEEMEAVGRDLHLHSMALRSAQSRVRGVIPAATAPPVEFDWRSHNGRSY
VISVKFQGACGICTCFSTTAAVESAICIATQTSPQIIQGVEVPALSEAQM
FYCGAASQDRICASGWFLPAALAYLQNTGVAPYSYFPYESGDQPCLIQPG
WWESVVTKIIGSTKLTAPDEIKSWIATRGPVAIMMVAYEDLFTYKEGIYH
PVSTNKLGVHSVCVVGYSDNKEALCKNSWSTQWGEDGYFWMAYGVCGMGS
SVHGINGLALVDGKPLSPRRPVA
>PoyI (Fe(II)/alpha-ketoglutarate-dependent
oxygenase)
(SEQ ID NO: 19)
MIQNPGYSLPVERVNELSREQFRKDYLAHSRPVVVIGGVREWPALKRWEL
ETLTERLQDRIVEIASTAKGIFSYDLESPRAKYEYMAFSDAAALVAQGQR
DAQYYIMQLSIEHYFSELRDDILRLDLLSGEACSPHFWLGGADLVTPLHW
DNLHNLYGQVRGRKRFTLFAPAEHDNLYPYPATALYGHMSYANPEASEQW
PKLRDAERFECILAPGDLLFLPAFWWHHVRSLELAISVNFWWVPGLSGCF
VPAFLRILRMAYRRERLIGLGAPVSTFPGGPIGAARSALRNGQTSFAMLF
AASALEKTIRARCYAVGIDDWEDATPRPIEVLDAELAACGAYPPDLDRAR
LGSWTHAINRVVDGDSETALSVAEATTIVDEIRMFVTDMH
>PoyJ (putative transporter/hydrolase)
(SEQ ID NO: 23)
MSQSNRMGTTPADATEAGIDSSAAPAGDASADQEVAPGWVAVLFPNGEPL
VYALSIADERYATQWTVYRGPREGDLHEIEFFLELDPVAMGLGGELVRQR
SVLLCNGALDPVHYLSEAGGIRWEVRFESAEVTAILPDGSEQVVPRGEAQ
FIAADNVPGHKALIFAALACRKLLDQEVTVGLFLANQLAAVPYQMSPALD
LAAESGTWHRSSHLEEIHLDEHGLMIEGMVPTQGVRGWLERPGPPVPQWL
DEDQPVTVPLRYHYRDNTRFRLEDVTIPGPVTPIGATLSIPEGPGPFPAV
LFLSGSGTHDRHGIAGEIDMGTHEIMDFLAEQGLLGLRFDSRGAGTTKMG
EDTLVRGLDSEIADARACLEFLRARPEAAGGAVFLLGHSQGGTVALVLAE
QEGIALRGVVLMATMGRSIEDVITDQIVYMGKDIGLTDEQIEQQIQEVQE
AVELVKADHPWNPDNIPHYLLAMFRSPTWLKQLLLYRTVELITRLQCPLL
VCQGSKDFQVSAERDAELLVTAAQSAGGDCTYALFPNLDHLFKATAGEST
MAQYFDQTRHVDPEFLQRVAAWLSEHAS
>PoyK (unknown)
(SEQ ID NO: 21)
MLQTQCLKVPIIPGKEAEVKNWLAALSTRHQEVLEAITSEDIADEAMFYA
KEPSGEFLYLYSRAPDLAVAGAAFQKSQLPIDQEFKRISAECLDYRSAIR
LELLLSADSRNKYVYP
>ORF-1 (integrase; outside of poy gene cluster,
just downstream of poyI)
(SEQ ID NO: 25)
MAISFKGAHFPPEVILMGVRWYLAYPLSTRHVEELMAERGVHVDHSTVNR
WVVKYSPQLEAEFHRRKRAVWTSWRMDETYIKVKGEWKYLYRAVDKFGKT
FIDLLTEQRDEKAARKFLNKAIGRHGSVPEKITIDGSAANEAAIKSYNKD
HGMSIEIRQVKYLNNIVEQDHRGVKRITRPMLGFQSFDSAQSTLTGIELM
HMLRKGQLEDRGEQGLSVADQFYALAS
>ORF-2 (toxin-antitoxin system: toxin component;
outside of poy gene cluster, just upstream of
poyK)
(SEQ ID NO: 27)
MTGNEFIRRLRRLGRQRGVRVEFVPERGKGSHGTLYYGERLTIVRNPRDE
LKTGTLYAMLAQLGLSRADL
>M. marina ATCC 23134 precursor peptide; precursor
peptide marked in bold, marking includes GG-
cleavage motif
(SEQ ID NO: 29)
MTQQEILDRFGSLEKLITDTNFRNALKKDPRKALAQELSGVTIPDNVSLI
VHENTTNEMHIILLPDAEVSGEDMPDDDPMEVVLDKAMADKSFKDLLMID
PKGVLAKELPDFYVPDEFKVYFHENTATEWHLLIPSLETEDEDGELSEDE
LEAVAGGAGRRRRRRRRGPHIGRRRGGKGPRCRKRRFR
>D. baarsi DSM 2075 precursor peptide; precursor
peptide marked in bold, marking includes LG-
cleavage motif
(SEQ ID NO: 31)
MSSDNMAHSSAWAKVVAKAWADESYKNKLLSDPAAVLRAEGLAIPEGVRL
TVLENSATQIHLVLPVAPSDAADLEDAALGERLAAVI
>C. luteolum DSM 273 precursor peptide; precursor
peptide marked in bold, marking includes GG-
cleavage motif
(SEQ ID NO: 33)
MACNSDRSLLLSQPKTKDVPMEANEQQQALGKIIANAWADEGFKQQFIEN
PAEILRAEGISVPDGMMVNVMENTPTCMHIVLPQSPDIDLDGAALDALAG
GEYVLCSGGWCQQE
>N. punctiforme PCC 73102 precursor peptide 1;
precursor peptide marked in bold, marking includes
GG-cleavage motif
(SEQ ID NO: 35)
MSEQEQAQTRKNIEARIVAKAWKDEGYKQELLTNPKAIIEREFGVEFPAE
VSVQVLEENSTSLYFVLPISPVAIAQELSEEQLEAIAGGYMTTLASANAS
AKINPILPIRHSLVKTLR
>N. punctiforme PCC 73102 precursor peptide 2;
precursor peptide marked in bold, marking includes
AG-cleavage motif
(SEQ ID NO: 37)
MTQQEQAQTRQDIEARIIAKAWKDEAYKQELLTNPKAVIEREFGVEFPAD
VNVQVLEENPTSLHFVLPISPVAIAQELSEEELLALAAGVNYSAVTVAIV
KNTVKQNTNIITRAAVSVTALVTGASIGASSVHL
>Nostoc sp. PCC 7120 precursor peptide; precursor
peptide marked in bold, marking includes GG-
cleavage motif
(SEQ ID NO: 39)
MSEQTKTRKDVEAQIIVQAWKDEAYRQELLNNPKKIVEQEFGVQLPEGIT
VHVMEENASNLYFVIPARPNLEDVELSDEQLEAVAGGALWTLTLLLIPIA
HGALEEHNSRK
>Oscillatoria sp. PCC 6506 precursor peptide 1;
precursor peptide marked in bold, marking includes
GG-cleavage motif
(SEQ ID NO: 41)
MSTRKEAEEQLAIKALKDPSFREKLKANPKAVISSEFNTQVPDDLTIEVV
EETATKMYLVLPAPEAVEEELSEEQLEAVAGGGCWIAGSRGCGFVTRT
>Oscillatoria sp. PCC 6506 precursor peptide 2;
precursor peptide marked in bold, marking includes
GG-cleavage motif
(SEQ ID NO: 43)
MTSSTSQPEPMTREELQAKLIAKAWQDESFKQELLSNPTAVIAKEMGVDN
IPGITIQIVEETPTTYYLVLPSKPTDDTEELSDAELEAIAGGRRRGGSSR
VITNTPGVPGCN
>Azospirillum sp. B510 precursor peptide; pre-
cursor peptide marked in bold, marking includes
GG-cleavage motif
(SEQ ID NO: 45)
MTDQTQSAPMTRRDLEAKIVARAWSDDDFKAKFLADPKAMFEEHLGTKLP
ASLVMTAHEETADTIHFVIPAKPRIDLDELSDEDLEKVAGGVDIVTTITV
TAIISAGVGGAAFSAVATVLAAGGIRGVCAKW
>P. thermopropionicum SI precursor peptide; pre-
cursor peptide marked in bold, marking includes
GG-cleavage motif
(SEQ ID NO: 47)
MIESEKKPVIRKELKEQIIRKAQEDREFKKALVGNPKGAVEQLGVQLPED
VEVKVVEESAEVVYLVLPVNPGELTGEQLDNVAGGTGCSDVYSFPICVPT
YHDNTVPAPKAG
>Cleaved form of the PoyA precursor peptide
(SEQ ID NO: 48)
TGIGVVVAVVAGAVANTGAGVNQVAGGNINVVGNINVNANVSVNMNQTT
>Cleaved form of the M. marina ATCC 23134 pre-
cursor peptide
(SEQ ID NO: 49)
AGRRRRRRRRGPHIGRRRGGKGPRCRKRRFR
>Cleaved form of the D. baarsi DSM 2075 pre-
cursor peptide
(SEQ ID NO: 50)
ERLAAVI
>Cleaved form of the C. luteolum DSM 273 pre-
cursor peptide
(SEQ ID NO: 51)
EYVLCSGGWCQQE
>Cleaved form of the N. punctiforme PCC 73102 pre-
cursor peptide 1
(SEQ ID NO: 52)
YMTTLASANASAKINPILPIRHSLVKTLR
>Cleaved form of the N. punctiforme PCC 73102 pre-
cursor peptide 2
(SEQ ID NO: 53)
VNYSAVTVAIVKNTVKQNTNIITRAAVSVTALVTGASIGASSVHL
>Cleaved form of the Nostoc sp. PCC 7120 precursor
peptide
(SEQ ID NO: 54)
ALWTLTLLLIPIAHGALEEHNSRK
>Cleaved form of the Oscillatoria sp. PCC 6506
precursor peptide 1
(SEQ ID NO: 55)
WIAGSRGCGFVTRT
>Cleaved form of the Oscillatoria sp. PCC 6506
precursor peptide 2
(SEQ ID NO: 56)
SSRVITNTPGVPGCN
>Cleaved form of the Azospirillum sp. B510 pre-
cursor peptide
(SEQ ID NO: 57)
VDIVITITVTAIISAGVGGAAFSAVATVLAAGGIRGVCAKW
>Cleaved form of the P. thermopropionicum SI pre-
cursor peptide
(SEQ ID NO: 58)
TGCSDVYSFPICVPTYHDNTVPAPKAG
>UZ-HT15 his-tag alternative
(SEQ ID NO: 59)
MKHQHQHQHQHQHQQ
>S-tag
(SEQ ID NO: 60)
KETAAAKFERQHMDS
>target peptide sequence of degenerated oligo-
nucleotide Polytheo-For1
(SEQ ID NO: 68)
GIGVVVA
>target peptide sequence of degenerated oligo-
nucleotide Polytheo-For2
(SEQ ID NO: 69)
GAGVNQT
>target peptide sequence of degenerated oligo-
nucleotide Polytheo-Rev
(SEQ ID NO: 70)
VNMNQTT

### Example 9

### Identification of the Polytheonamide Biosynthetic Locus

Theonella swinhoei Y (morphotype with yellow interior) was collected in 2002 by hand during scuba diving at Hachijo-jima Island, Japan, at a depth of 15 m. Immediately after collection, specimens were shock-frozen in liquid nitrogen, followed by storage at −80° C. Metagenomic DNA was isolated from the sponge according to a previously published procedure (Gurgui and Piel, Methods Mol. Biol. 668 (2010) 247-264)). Because the isolated crude DNA contained contaminants that inhibit PCR, further purification was necessary, which was achieved by size-selection on low melting point agarose (Gurgui and Piel, Methods Mol. Biol. 668 (2010) 247-264). Approximately 0.5 μL of the obtained DNA was used in a 50 μL PCR mix that also contained 1 mM MgCl₂, 1 μM of each primer, 0.3 μM dNTPs, 6% DMSO, 1×BSA, and 3.75 U of Taq polymerase in 1× ThermoPol Reaction Buffer (New England Biolabs). A combined gradient-touchdown PCR reaction was used: 94° C. for 1 min, 16 cycles of: 94° C. for 30 sec, 50, 50.4, 51.5, 53.2, 55.1 or 57° C. for 30 sec (dT: −0.5° C./cycle), 72° C. for 30 sec. Then the following was cycled 22 times: 94° C. for 30 sec, 42, 42.4, 43.5, 45.2, 47.1 or 49° C. for 30 sec, 72° C. for 30 sec. Final incubation was at 72° C. for 10 min. The degenerate primers used in this PCR reaction were: Polytheo-For1 (for peptide sequence GIGVVVA (SEQ ID NO: 68), for all primer sequences see Table 1) and Polytheo-Rev (for peptide sequence VNMNQTT (SEQ ID NO: 70)). PCR mixtures were loaded on a 2% agarose gel, and an approximately 150 bp DNA fragment was excised and extracted. Approximately 0.75 μL of the purified DNA was used in a semi-nested PCR reaction with the following primers: Polytheo-For2 (for peptide sequence GAGVNQT (SEQ ID NO: 69)) and the reverse primer Polytheo-Rev (see above). All other PCR conditions were identical to those described above. The PCRs generated a single fragment with an approximate size of 100 bp, which was purified directly with a PCR purification kit (Fermentas). The fragment obtained during the two rounds of PCR was then ligated into pBluescript SKII (+) and transformed into chemically competent E. coli XL1Blue cells. Plasmid DNA was isolated from positive clones and end-sequenced with the M13 forward primer.

### Results:

Sequencing revealed a succession of codons that precisely corresponded to an unprocessed polytheonamide precursor, thus supporting its ribosomal origin.

### Example 10

### Isolation of the Polytheonamide Biosynthetic Genes

To isolate the biosynthetic gene cluster, a previously constructed ˜60,000 member metagenomic cosmid library of Theonella swinhoei (Piel et al., Proc. Natl. Acad. Sci. U.S.A. 101, (2004) 16222-16227) was screened by using a pool-dilution protocol as described previously (Hrvatin and Piel, J. Microbiol. Methods 68 (2007), 434-436). The PCR conditions were: 95° C. for 5 min, repeat 35 times: 95° C. for 30 sec, 59° C. for 30 sec, 72° C. for 30 sec. Final incubation was at 72° C. for 5 min. The following primers were used: PolytheoXFor1/PolytheoXRev1 or PolytheoXFor2/PolytheoXRev2 (Table 1). The cosmid pTSMAC1 was isolated, which contained a part of the poy locus. To isolate the remaining poy region, a primer walking strategy was employed using the initial library and, additionally, a ca. 860,000 clone fosmid library previously constructed from the same sponge (T. Nguyen et al., Nat. Biotechnol. 26, 225 (2008)). Only one additional poy-positive pool of ca. 1000 clones was identified. However, it was repetitively lost during the enrichment process. Plasmid DNA was isolated from this pool and 2 μL were used as a template for long-range PCR. The following primers were used based on sequences of the cosmid vector and the pTSMAC1 insert: pWEB For and Up-PolyTheo-Rev2 (Table 1). A 2 μL aliquot of this PCR reaction was used in a second semi-nested PCR with the primers: pWEB For and Up-PolyTheo-Rev1. Each 50 μL PCR reaction mix also contained 1 mM MgCl₂, 0.5 μM of each primer, 0.5 μM dNTPs, 5% DMSO, and 1 U of DyNAzyme EXT DNA Polymerase (Finnzymes). The conditions used were: 94° C. for 1 min, 15 cycles of: 94° C. for 30 sec, 59° C. for 30 sec, 68° C. for 25 min. Then the following was cycled 20 times: 94° C. for 30 sec, 59° C. for 30 sec, 68° C. for 25 min+20 sec/cycle. Final incubation was at 68° C. for 25 min. An approximately 7 kb DNA fragment was isolated after agarose gel electrophoresis, ligated into pGEM-T easy (Promega), and the resulting plasmid was introduced into E. coli XL1-Blue and sequenced. The sequence was confirmed by re-sequencing regions directly amplified from the metagenomic DNA.

### Results:

To identify the surrounding DNA region, in total 980,000 clones of a library of T. swinhoei total DNA (Piel et al., Proc. Natl. Acad. Sci. U.S.A. 101, (2004) 16222-16227) were screened in a pool-dilution strategy (Hrvatin and Piel, Microbiol. Methods 68, (2007) 434-436), yielding a single cosmid pTSMAC1. The few other clones detected were repeatedly lost during isolation as indicated above. To expand the upstream sequence, a 7 kb portion was amplified directly from the partially enriched pool by long-range PCR, using primers based on sequences of the cosmid vector and the pTSMAC1 insert. The authenticity of the amplified region was subsequently confirmed by repeated PCR and sequencing using metagenomic DNA.

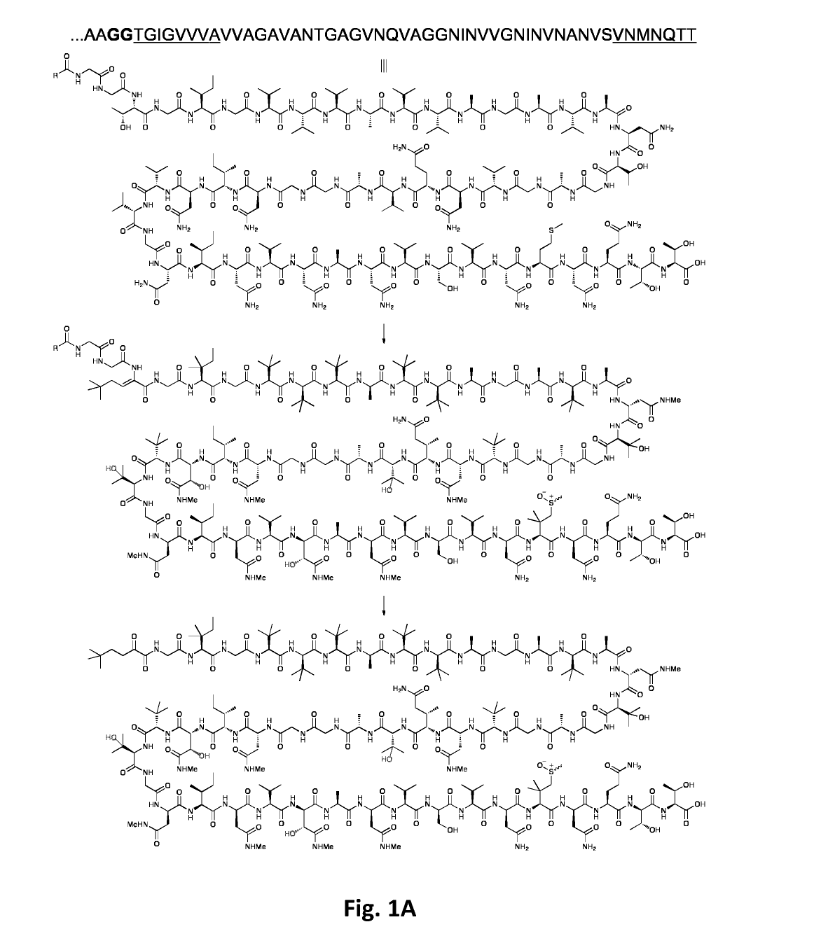

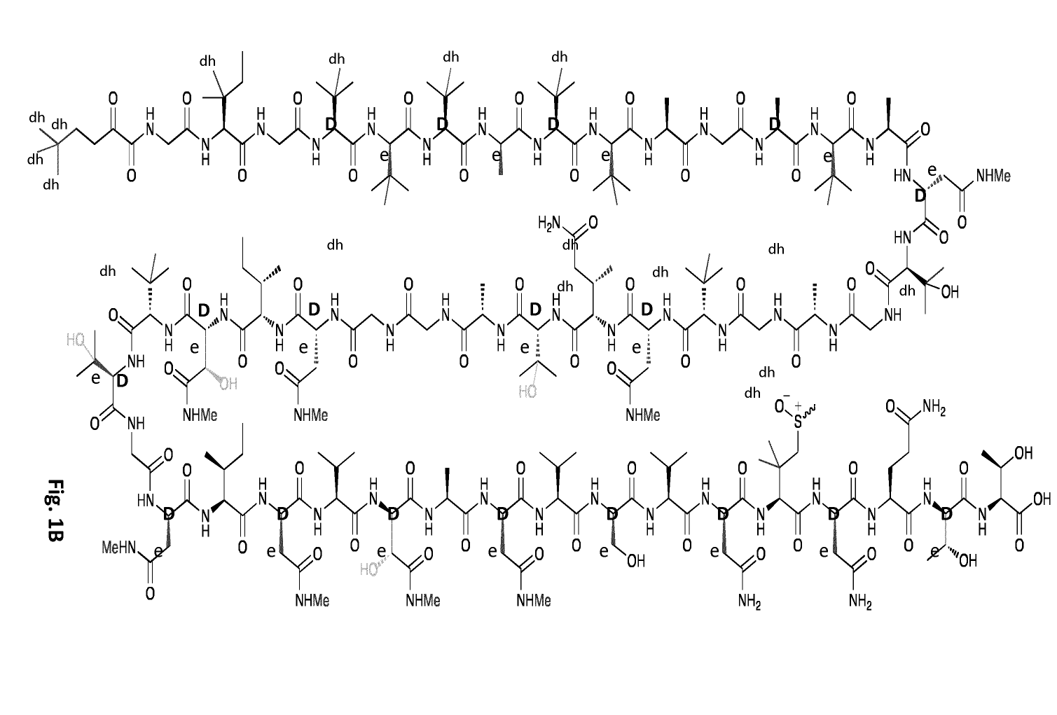

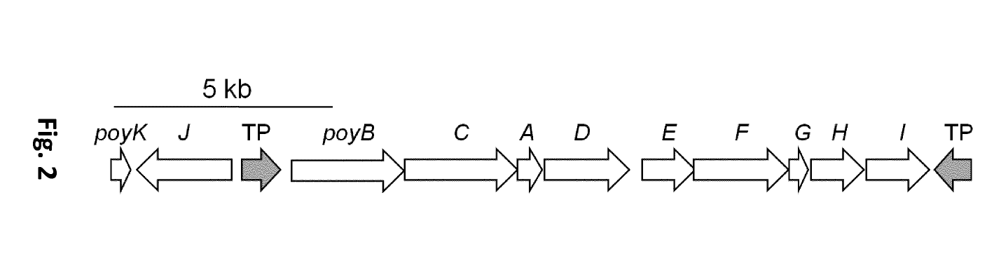

The assembled DNA region contained eleven additional genes, clustered around the initially identified open reading frame (ORF) (FIG. 2). Nine ORFs, which we termed poy genes (poyA-I), form an operon, as apparent from the short or often absent intergenic regions. This polycistronic architecture, as well as the presence of Shine-Dalgarno motifs and lack of detectable introns, suggests a bacterial endosymbiont as the origin of the cloned region. Beyond the gene cluster, the presence of an upstream prokaryotic hicAB-type toxin-antitoxin system, numerous genes and gene fragments resembling bacterial transposition elements, and two downstream genes encoding a polyketide synthase of as-yet unknown function further support this hypothesis (Table 5). The 3′ terminus of poyA consists of 48 codons that match a complete polytheonamide precursor. Remarkably, the encoded sequence suggests the three 3-hydroxyvaline units to originate from two different residues: residue 16 from threonine (Thr) by C-methylation and residues 23 and 31 from valine (Val) by hydroxylation (FIG. 1B). In addition to the propeptide-encoding core region, an unusually long 5′ leader sequence in poyA was identified that exhibits homology to nitrile hydratases. This region does not resemble any component of characterized ribosomal pathways. As already indicated hereinabove, however, Haft and coworkers (Haft et al. (BMC Biol. 8:70 (2010)) recently discovered similar leaders in several taxonomically diverse bacteria by in silico genome analysis and postulated the existence of a new natural product family.

### Example 11

### Cloning of Constructs for Protein Expression

The gene poyA was codon-optimized for E. coli expression by DNA2.0 (Menlo Park, Calif.), with optimized sequence as indicated in Table 1 (SEQ ID NO: 90). The codon-optimized construct was amplified using primers PT-opt-F and PT-opt-R (Table 1). The PCR product was then digested with NdeI and HindIII and cloned into pET28b (EMD Biosciences, Darmstadt, Germany) using the same restriction sites. This plasmid was used for expression tests of N-terminally His₆-tagged PoyA (Nhis-PoyA). For co-expression trials, Nhis-poyA was excised from this construct using NcoI and HindIII and cloned into a pETDUET-1 plasmid (EMD Biosciences, Darmstadt, Germany) already containing the genes poyB, poyC, poyD, or poyE. Genes poyB/C/D/E/F were PCR-amplified with primers SAM1-F and SAM1-R, SAM2-F and SAM2-R, SAM3-F and SAM3-R, and NMethT-F and NMethT-R, and Lanth-F and Lanth-R, respectively (Table 1). The gene poyE was additionally codon-optimized for E. coli expression by DNA2.0 (Menlo Park, Calif.) with the final sequence as indicated in Table 1 (SEQ ID NO: 92). The codon-optimized poyE gene was amplified with primers NMethT-opt-F and NMethT-opt-R (Table 1) and cloned into various pETDUET-1, pCDFDUET-1, and pCOLADUET-1 constructs. To create the co-expression construct for Nhis-poyA and poyF, primers PT-opt-F3 and PT-opt-R were used to amplify poyA, which was then digested with EcoRI and HindIII into the pETDUET-1 construct already containing poyF. For some constructs, one or more codons were silently mutated for ease of cloning and are represented in the corresponding primer sequences. Additionally, poyD was amplified and ligated into pCDFDUET-1 (EMD Biosciences, Darmstadt, Germany) as in pETDUET-1 to allow for triple expressions of Nhis-poyA, poyD, and either poyB, poyC, poyE, or poyF. A shortened version of Nhis-poyA containing roughly half of the polytheonamide core sequence, Nhis-poyA121, was cloned into pET28b using primers PT-opt-F and PTopt-half-R (Table 1) and subsequently cloned into pETDUET-1 and pCDFDUET-1 dual expression constructs along with poyD as previously described. As a control for epimerization experiments, an Nhis-poyA construct truncated at residue 101 named Nhis-poyA101 was made harboring only the first five residues of the polytheonamide core sequence (which does not contain epimerized amino acids in the mature form). Primers PT-opt-F and PT-OPT_R_ADD_{—}1_{—}9 (Table 1) were used to clone Nhis-poyA101 in pET28b and was subsequently cloned into a pETDUET-1 dual expression construct along with poyD as previously described.

### Example 12

### Improved Heterologous Expression of Multiple Polytheonamide (Poy) Genes

### Methods:

Some hints concerning the function of the particular genes of the poy-cluster and in the neighboring genes could be already obtained by sequence similarity studies as indicated herein above and summarized below in Table 4. However, to generate polytheonamides from proteinogenic residues, 4 hydroxylations, 18 epimerizations, and at least 21 methylations are necessary (FIG. 1B). Considering the large number of posttranslational modifications, astonishingly few enzyme candidates were identified for these steps. These are PoyB, PoyC, and PoyD, homologous to members of the radical S-adenosylmethionine (rSAM) superfamily (Sofia et al., Nucleic Acids Res. 29, (2001) 1097-1106; Frey et al., Crit. Rev. Biochem. Mol. Biol. 43 (2008), 63-88); PoyE, homologous to SAM-dependent methyltransferases; PoyI, homologous to Fe(II)/α-ketoglutarate oxidoreductases; and PoyF, homologous to the dehydratase domain of LanM-type lantibiotic synthetases (You and van der Donk, Biochemistry 46, (2007) 5991-6000). Besides these six enzymes the cluster encodes other proteins likely involved in regulation, transport, and proteolytic removal of the leader region (PoyJGH) based on homologies (see herein above and Table 5, below). No homology was found for PoyK and it is unclear whether it belongs to the pathway. The limited number of maturation factors suggests that individual enzymes convert positionally and structurally diverse residues. For example, C-methylation occurs on at least five different units, while at least four types of residues are epimerized. Conversely, identical residues are processed in different ways, such as Val and asparagine (Asn), which each appear as three structural variants. Further data characterizing how this biosynthetic machinery reconciles substrate promiscuity with regiospecificity could be obtained by the co-expression experiments as described below.

### Codon-Optimization and Cloning

For an improved heterologous expression of the polytheonamide (poy) genes, in particular to increase the amount of soluble Nhis-PoyA in the culture product, the protocol described in Examples 3 and 4 above has been further modified. The gene poyA has been codon-optimized, as already indicated above in Example 1, for E. coli expression by DNA2.0 (Menlo Park, Calif.; Table 1, SEQ ID NO: 90). The codon-optimized construct was amplified using primers PT-opt-F and PT-opt-R (Table 1). The PCR product was then digested with NdeI and HindIII and cloned into pET28b (EMD Biosciences, Darmstadt, Germany) using the same restriction sites. This plasmid was used then for expression tests of N-terminally His₆-tagged PoyA (Nhis-PoyA).

For co-expression trials Nhis-poyA was excised from this construct using NcoI and HindIII and cloned into a pETDUET-1 plasmid (EMD Biosciences, Darmstadt, Germany) already containing the genes poyB, poyC, poyD, or poyE. Genes poyB/C/D/E/F were PCR-amplified with primers SAM1-F and SAM1-R, SAM2-F and SAM2-R, SAM3-F and SAM3-R, and NMethT-F and NMethT-R, and Lanth-F and Lanth-R (Table 1), respectively. The gene poyE was additionally codon-optimized for E. coli expression by DNA2.0 (Menlo Park, Calif.; Table 1; SEQ ID NO: 92)

The codon-optimized poyE gene was amplified with primers NMethT-opt-F and NMethT-opt-R (Table 1) and cloned into various pETDUET-1, pCDFDUET-1, and pCOLADUET-1 constructs.

To create the co-expression construct for Nhis-poyA and poyF, primers PT-opt-F3 and PT-opt-R were used to amplify poyA, which was then digested with EcoRI and HindIII into the pETDUET-1 construct already containing poyF. For some constructs, one or more codons were silently mutated for ease of cloning as represented in the corresponding primer sequences.

### Protein Expression:

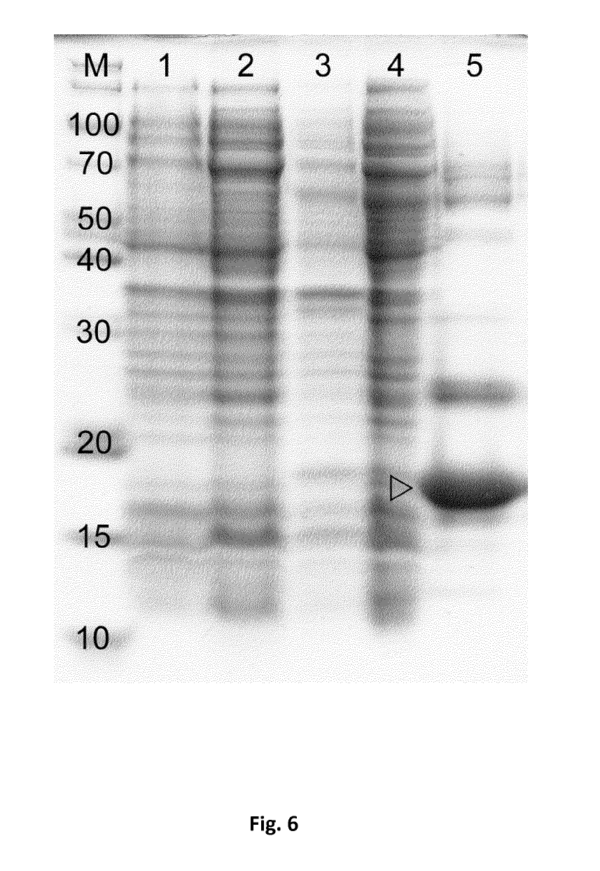

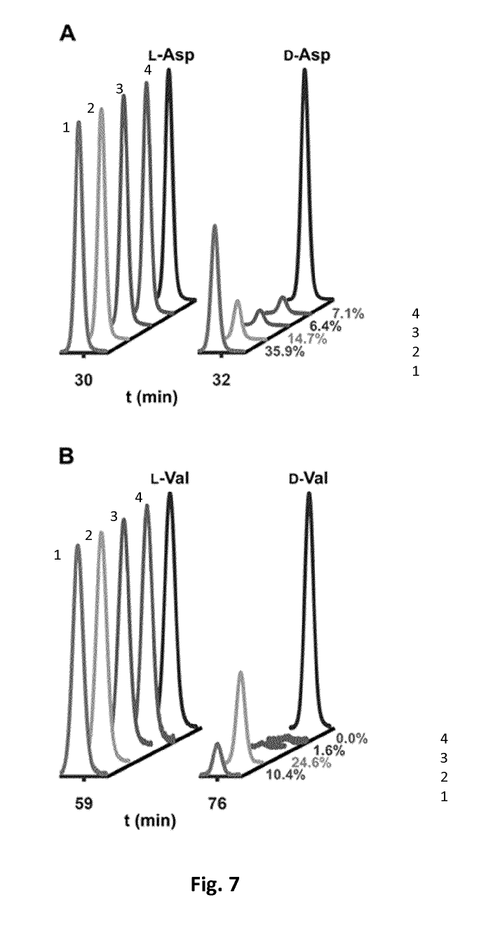

Attempts to express soluble Nhis-PoyA in a variety of E. coli BL21(DE3) derivatives produced only small amounts of soluble product. Induction was finally achieved (visible by Coomassie-stained SDS-PAGE) in BL21(DE3)star pLysS (EMD Biosciences, Darmstadt, Germany) in TB medium at 37° C. for three hours post IPTG induction (FIG. 6). However, up to 6.0 L cultures expressing Nhis-PoyA either at 37° C. for three hours or at 16° C. for 18 hours did yield only small amounts of insoluble protein. Nhis-PoyA was successfully purified under denaturing conditions with Profino Ni-NTA resin (Macherey-Nagel, Duren, Germany) according to manufacturer's specifications. Protein was visualized by Coomassie-stained SDS-PAGE (FIG. 6) and Western Blot.

Vectors expressing Nhis-poyA and either poyB/C/D/E/F were induced in E. coli BL21(DE3)star pLysS at 16° C. for 18 hours. IPTG (1 mM final concentration) was added to cooled cultures grown in TB medium at 37° C. to an OD₆₀₀ ˜1.5-2.0. Nickel chromatography was performed as per manufacturer's instructions. Culture sizes ranged from 200 mL to 4.0 L TB medium. Soluble Nhis-PoyA was only produced in co-expression cultures with PoyD and not with PoyB/C/E/F.

Addition of the poyD-pCDFDUET-1 expression vector with the pETDUET-1 co-expression constructs described above allowed for all triple expression combinations in BL21(DE3)star pLysS. All expressions were performed as described above, and all yielded soluble Nhis-PoyA. The resulting proteins were dialyzed into 50 mM potassium phosphate, pH 7.0 using 3.5K MWCO Snakeskin dialysis tubing (Thermo Scientific, Dreieich, Germany) and flash-frozen in liquid nitrogen prior to storage at −80° C. Samples were subsequently subjected to MALDI-TOF analysis to determine whether any modifications were detectable with Nhis-PoyA.

Co-expressions with poyE did not yield any visibly induced PoyE by Coomassie-stained SDS-PAGE. Higher induction was achieved in co-expressions harboring codon-optimized poyE in a plasmid combination of Nhis-poyApoyD-pETDUET-1, poyFpoyE-pCDFDUET-1, and poyE-pCOLADUET-1. In these expressions, PoyF was not visibly detectable by Coomassie-stained SDS-PAGE. Expressions were performed at 16° C. in BL21(DE3)star pLysS for 1, 3, and 7 days. In addition, a coexpression of poyE in a plasmid combination of Nhis-poyA121_poyD-pCDFDUET-1 and poyF_poyE-pCOLADUET-1 was performed at 16° C. in BL21(DE3)star pLysS for 3 days.

Purifications used for subsequent amino acid analysis were further purified on a hydrophobic column. Nickel column elution fractions were dialyzed into 50 mM potassium phosphate, 0.5 mM EDTA, and 1.0 M (NH₄)₂SO₄, pH 7.0 and loaded by gravity flow onto Toyopearl Phenyl-650M resin (TOSOH Bioscience GmbH, Stuttgart, Germany) in an approximate ratio of 1 mg of protein per 1 mL resin bed volume. A 0.1 M (NH₄)₂SO₄ step gradient was run by gravity flow from 1.0 M (NH₄)₂SO₄ to 0 M (NH₄)₂SO₄ buffer. The column was run at room temperature and each step volume was 2× the resin bed volume. Pure fractions were combined and dialyzed into 50 mM potassium phosphate, pH 7.0.

### Detection of Epimerized Amino Acids

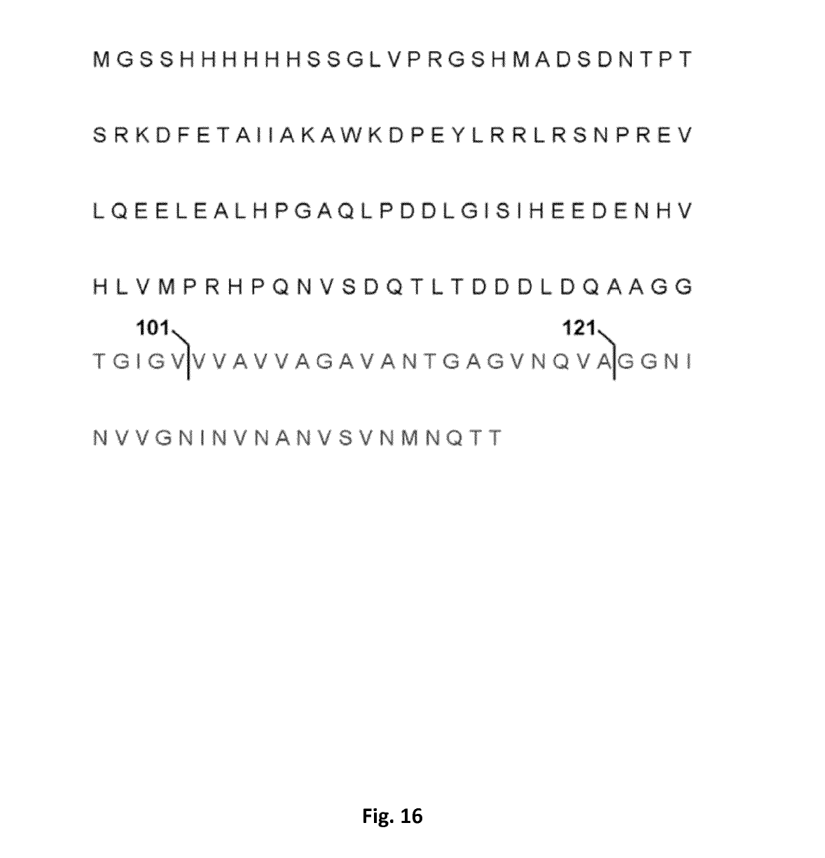

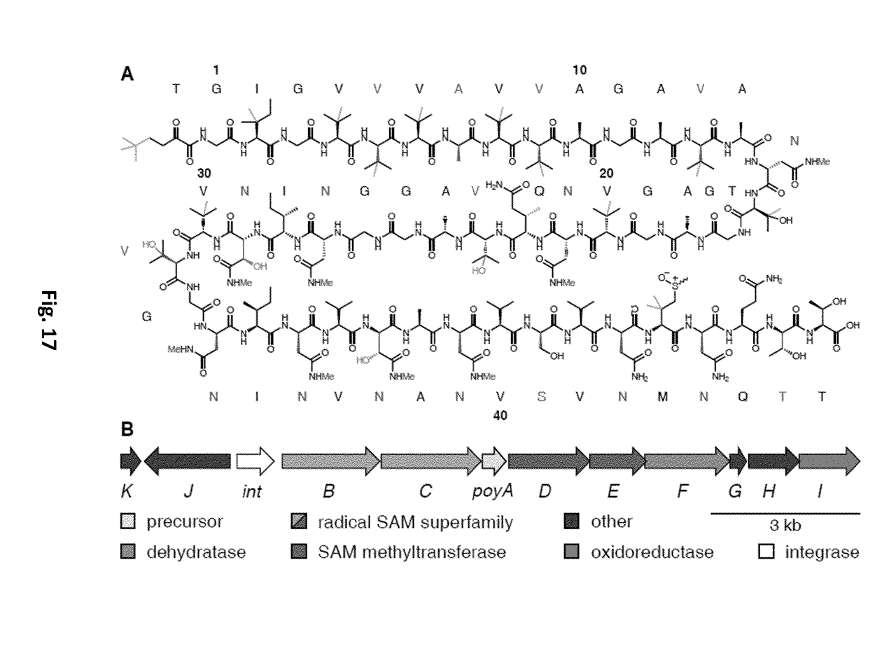

The protein concentration of the resulting sample from coexpression of Nhis-poyA with poyD, Nhis-poyA121 with poyD, Nhis-poyA101 with poyD, and Nhis-poyA101 (see FIG. 16 for the sequences of Nhis-poyA121 and Nhis-poyA101) alone was determined by Bradford assay using a standard curve derived from bovine albumin. A portion of the Nhis-poyA with poyD elution fraction (875 μL) was desalted with a vivaspin 500 5K MWCO column. The desalted protein (120 μg) was hydrolyzed in 6N HCl (600 μL) at 110° C. for 18 hours. During the hydrolysis all asparagine residues are converted to aspartic acid. The HCl solution was evaporated under a stream of argon, and the residue was re-evaporated from MilliQ grade water (2×300 μL) in a speedvac to remove residual HCl.

The hydrolyzed material was dissolved in MilliQ grade water (25 μL), 1N NaHCO3 (10 μL) and Na-(2,4-dinitro-5-fluorophenyl)-L-valinamide (L-FDVA, 50 μL, 1% in acetone) and then heated to 42° C. for 1 hour. The mixture was neutralized with 2N HCl (10 μL) followed by evaporation under a stream of Ar.

The mixture was redissolved in 600 μL 1:1 CH3CN/H2O and 20 μL were injected for HPLC analysis. The HPLC was carried out using a Knauer Eurosphere II-5 Phenyl, 5μ, 250×4 mm column using a flow rate of 1 mLmin⁻¹. HPLC conditions used to detect L-FDVA-derivatized Asp and Val utilized mobile phases A consisting of acetonitrile+0.1% TFA and B with dH₂O+0.1% TFA.

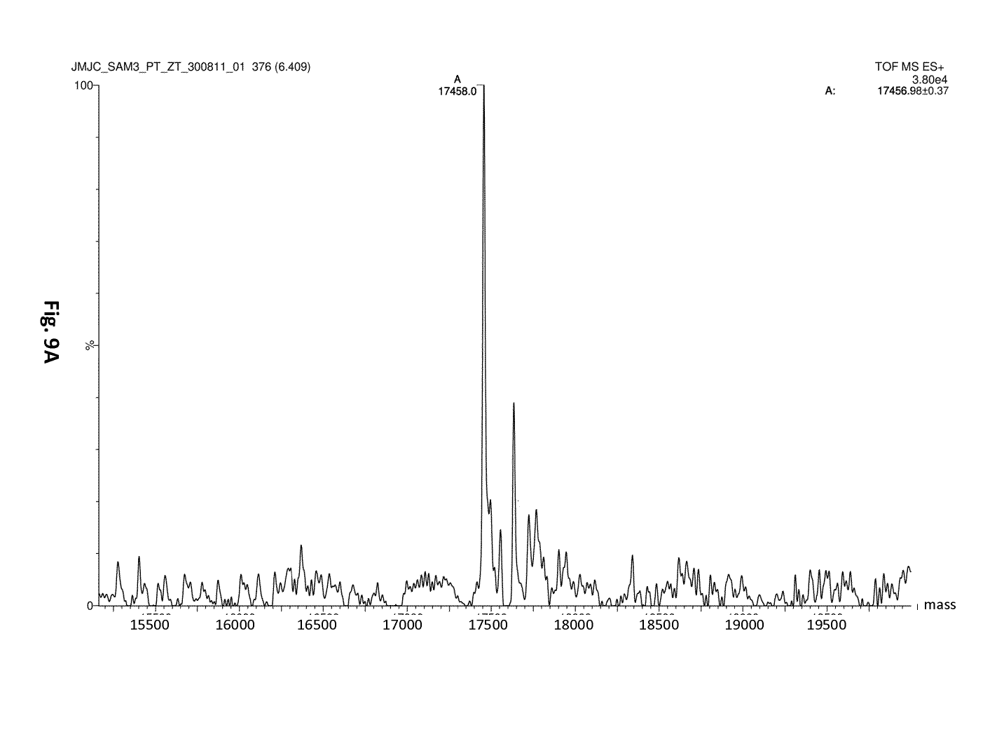

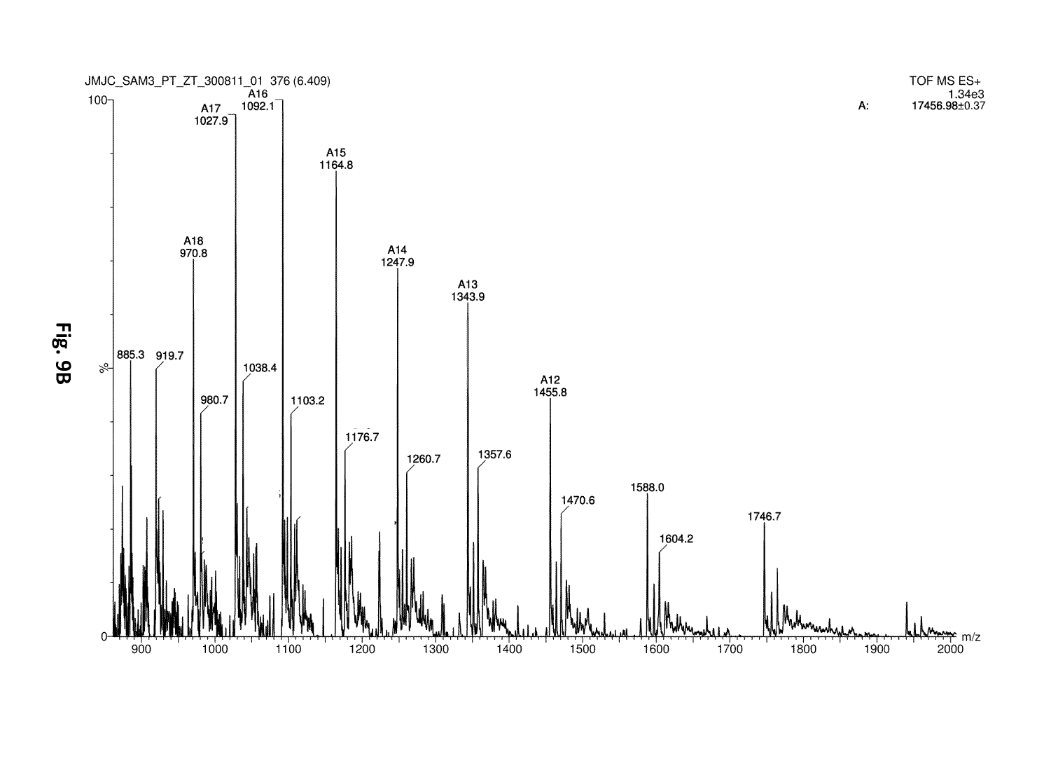

A gradient of 75% B to 65% B (60 minutes), 65% B to 55% B (90 minutes), 55% B to 30% B (110 minutes), and 30% B (120 minutes) was used. The peaks corresponding to L-FDVA-derivatized Asp were determined by ESI-MS (negative ion mode, [M-H]− m/z 412.1 (L-Asp), see FIG. 8A; m/z 412.1 (D-Asp), See FIG. 8B), photodiode array UV spectra (λmax, 335 nm and 411 nm), and comparison of retention times (29.79 min. (L-Asp); 31.71 min. (D-Asp)) to authentic material (29.82 min. (L-Asp); 31.75 min. (D-Asp). The peaks corresponding to L-FDVA-derivatized Val were determined by ESI-MS (negative ion mode, [M-H]− m/z 396.2 (L-Val), see FIG. 8C; m/z 396.2 (D-Val), see FIG. 8D), photodiode array UV spectra (λmax, 335 nm and 411 nm), and comparison of retention times (58.74 min. (L-Val); 76.01 min. (D-Val)) to authentic material (58.81 min. (L-Val); 76.08 min. (D-Val).

A similar procedure was used for the identification of D/L-Asp and D/L-Val in protein expressions of Nhis-poyA121 with poyD, Nhis-poyA101 with poyD, and Nhis-poyA101 alone.

### Mass Spectrometry

For direct analysis of intact PoyA variants and their tryptic digests, samples were desalted into 80:20 acetonitrile:water containing 0.1% formic acid using C4 ZipTips® (Millipore, Billerica, Mass.) following the manufacturer's standard instructions. Electrospray ionization-mass spectrometry of ZipTip-purified samples was performed on a Waters (Altrincham, UK) Synapt High Definition Mass Spectrometer (HDMS)—a hybrid quadrupole/ion mobility/orthogonal acceleration time of flight (oa-TOF) instrument.

Samples were infused into the standard nano-electrospray (z-spray) source. The capillary of the ESI source was typically held at voltages between 1.3 and 1.5 kV, with the source operating in positive ion mode. A sample cone voltage of 35 V was used. The trap T-wave collisional cell contained argon gas held at a pressure of 2.5×10-2 mbar. The oa-TOF-MS was scanned over a range of m/z 200-3000 at a pressure of 1.8×10-6 mbar. For MS/MS measurement of trypsinized Nhis-PoyA the quadrupole was set to transmit m/z 1364 ([M+5H]5+) and the collision energy in the trap region of the instrument raised to 22.3 V. The instrument was operated using MassLynx (version 4) software. Multiply charged electrospray spectra were deconvoluted using MaxEnt.

Tryptic digestion was performed on aliquots (50 μl) of Nhis-PoyA containing TPCK-treated trypsin (1 μM; Sigma-Aldrich, Poole, UK) incubated at 37° C. for 3 hours.

For LC/MS analyses, Nhis-PoyA samples were either measured intact or following trypsin digestion. For intact protein measurements an aliquot (20 μl) of Nhis-PoyA in 50 mM potassium phosphate was transferred into polypropylene sample vials (Dionex, Surrey, UK) for direct analysis. Samples (1-5 μl) were analysed by capillary reversed-phase-nanoLC with subsequent MS and MS/MS detection.

Chromatographic separation was performed using an Ultimate 3000 nano-HPLC system (Dionex, Surrey, UK) equipped with an autosampler. Samples were concentrated on a (300 μm i.d.×5 mm) trapping column packed with C18 PepMap300 (Dionex, Surrey, UK) using mobile phase A: Water/Acetonitrile (95:5, v/v) with 0.1% formic acid delivered at 25 μl min-1. The trapping column was switched in-line with the analytical column after a 3 min loading time. Chromatographic separation was performed using a reverse phase C18 column (length 15 cm×75 μm i.d., 3 μm Jupiter resin, manufactured ‘in-house’).

Samples were eluted using a linear gradient of B, a mixture of acetonitrile/water (95:5) with 0.1% formic acid, in buffer A, as follows: from 0% to 95% of buffer B in 20 min, held at 95% buffer B for 5 min, followed by 15 min re-equilibration with buffer A at a constant flow rate of 0.2 μl min⁻¹. The nanoLC was interfaced with an LTQ FT-Ultra mass spectrometer (ThermoFisher, Bremen, Germany) which was operated in positive ion mode and equipped with a standard Thermo nanospray ion source. MS instrumental conditions were optimized using a denatured horse heart myoglobin standard for intact protein work and a Substance P standard for peptide work. The heated capillary temperature was set at 275° C., the spray voltage was set at 1.6 kV and the capillary voltage was held at 43 V. Data were acquired using the FTICR in full scan mode (m/z 300-2000), or by trap selection of precursor ions. Electron capture dissociation (ECD) was performed with a relative electron energy setting of 1-4% and a reaction time of 120-300 ms. Collision-induced dissociation (CID) with helium gas was carried out in the linear ion trap using a relative energy setting of 20% and a reaction time of 30 ms. Fragments were subsequently transferred and measured in the ICR cell. A mass resolving power of 100,000 at m/z 400 was used and Automatic Gain Control (AGC) was applied in all data acquisition modes. Data were processed using ThermoFisher Xcalibur software. MALDI-TOF-MS spectra were recorded on a Bruker autoflex II TOF/TOF time of flight mass spectrometer.

### Results:

After codon-optimization and co-expression trials using various gene combinations, it was found that protein yields and solubility of PoyA dramatically improved in the presence of the rSAM protein PoyD (FIG. 6). PoyD exhibits close similarity to only a small number of uncharacterized proteins mostly from hypothetical proteusin gene clusters. To take use of the co-expression effect of PoyD, the poyD coding sequence was amplified and ligated into pCDFDUET-1 (EMD Biosciences, Darmstadt, Germany) as in pETDUET-1 to allow for triple expressions of Nhis-poyA, poyD, and either poyB, poyC, poyE, poyF or poyI.

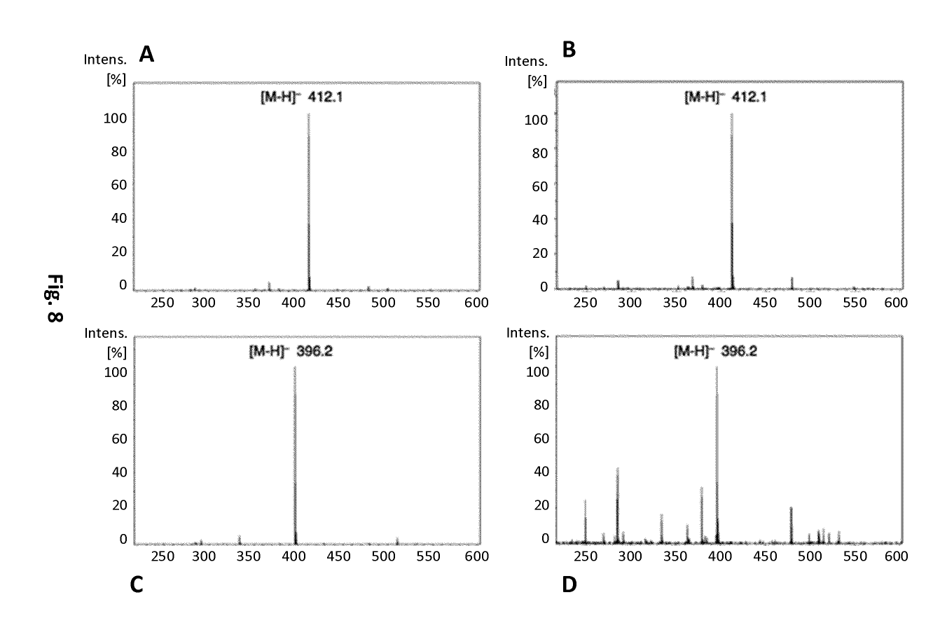

Mass spectrometric (MS) analysis of purified PoyA obtained from co-expression with PoyD did not reveal a mass shift or apparent modification of the protein sequence. Further analysis of PoyA involving acid hydrolysis, derivatization, and chromatographic separation of MS-verified amino acids revealed the presence of epimerized asparagines and valines within the PoyA core sequence, confirming that PoyD is capable of epimerizing most, and perhaps all, of observed D-amino acids present in polytheonamides A and B (FIGS. 7, 8 and 16).

The unidirectional L- to D-amino acid epimerization observed with PoyD is in contrast to known non-radical amino acid racemases, which generate an equilibrium mixture of epimers (McIntosh et al., Nat. Prod. Rep. 26 (2009), 537-559).

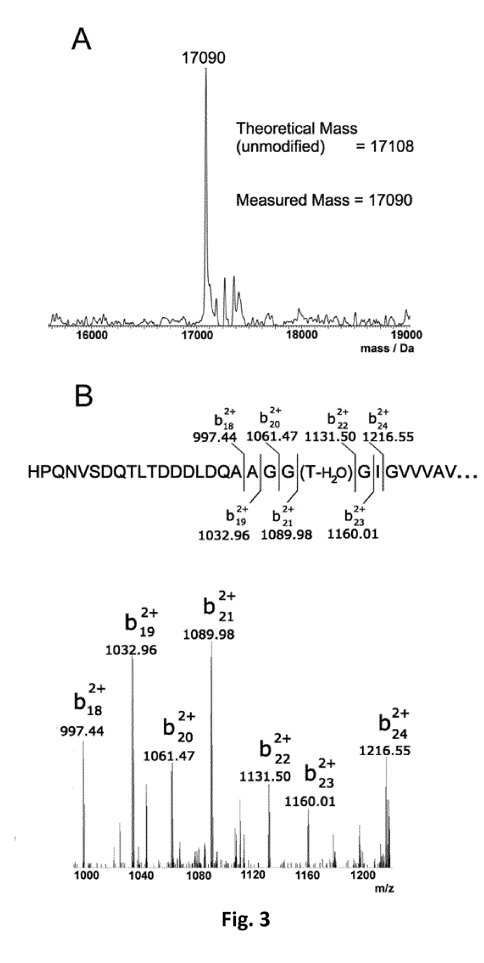

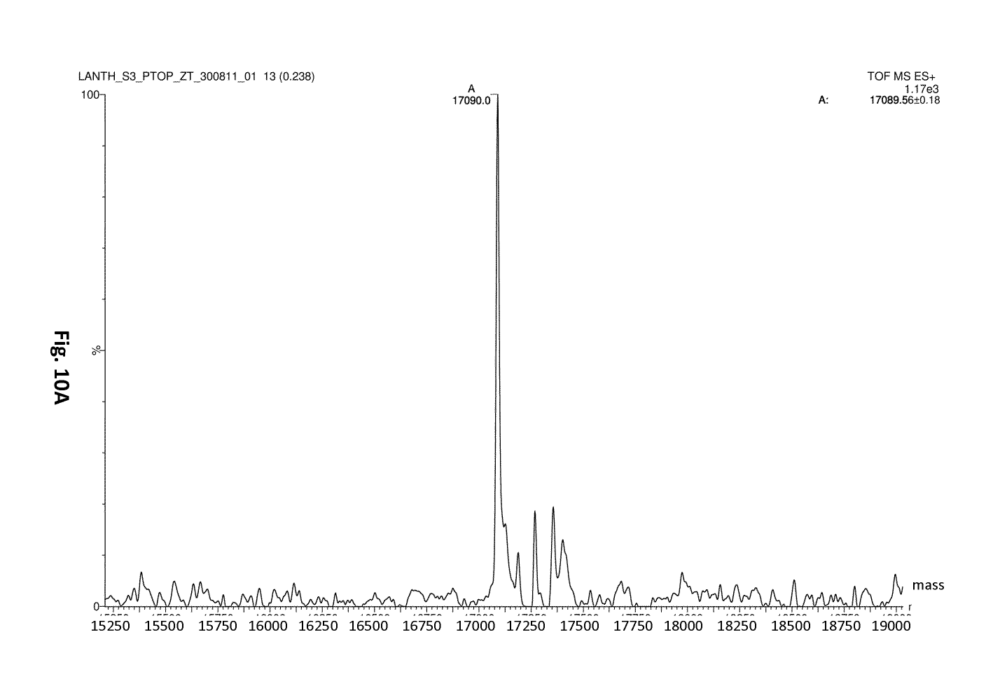

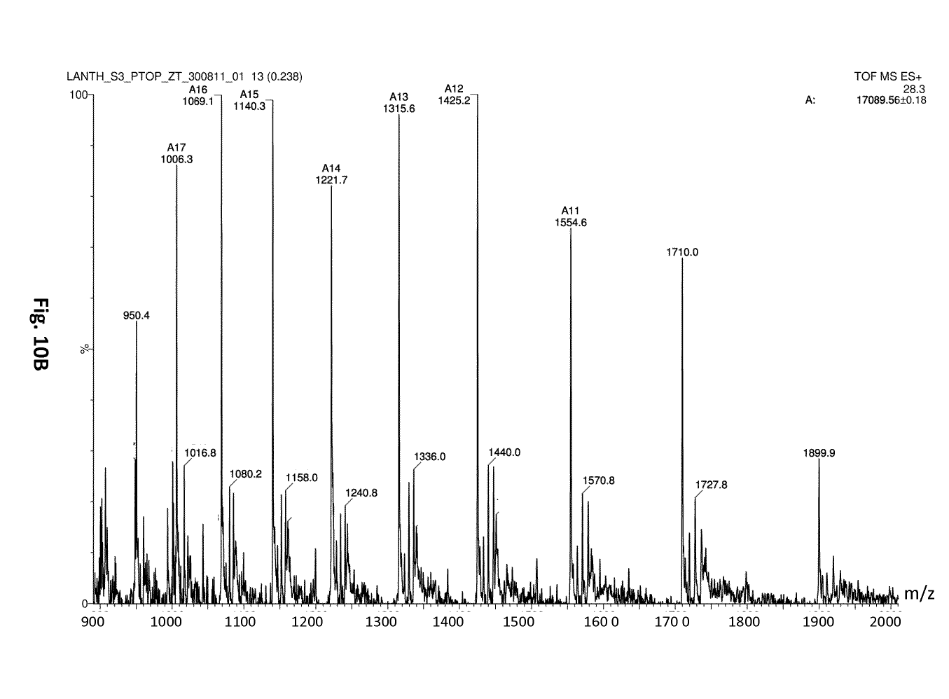

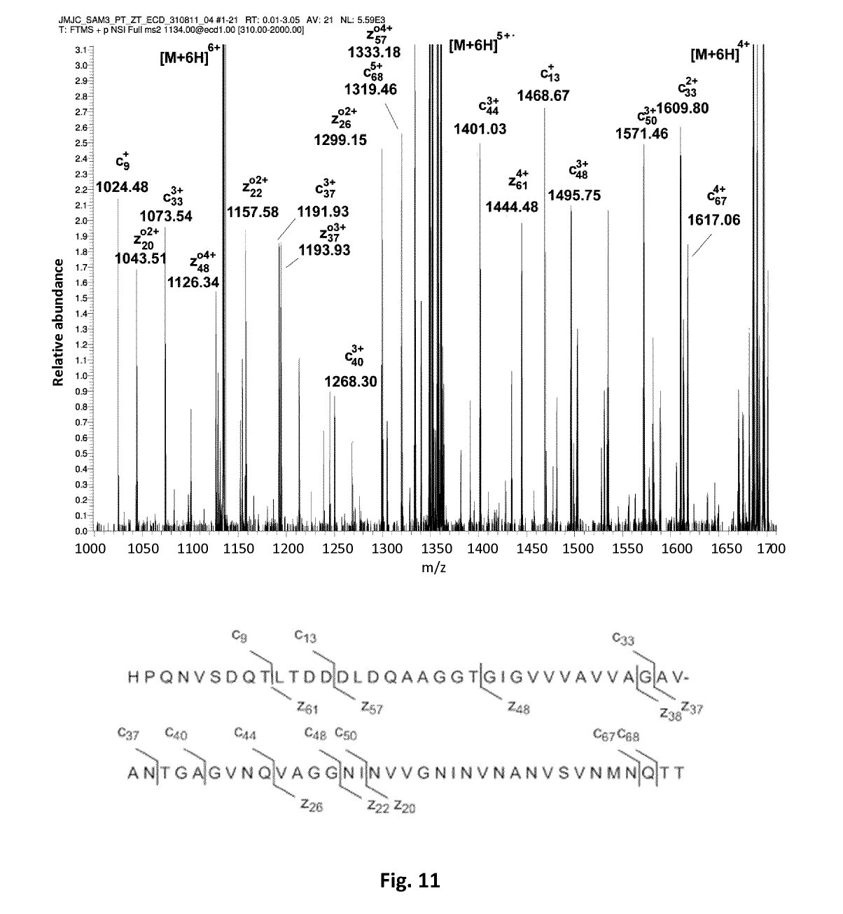

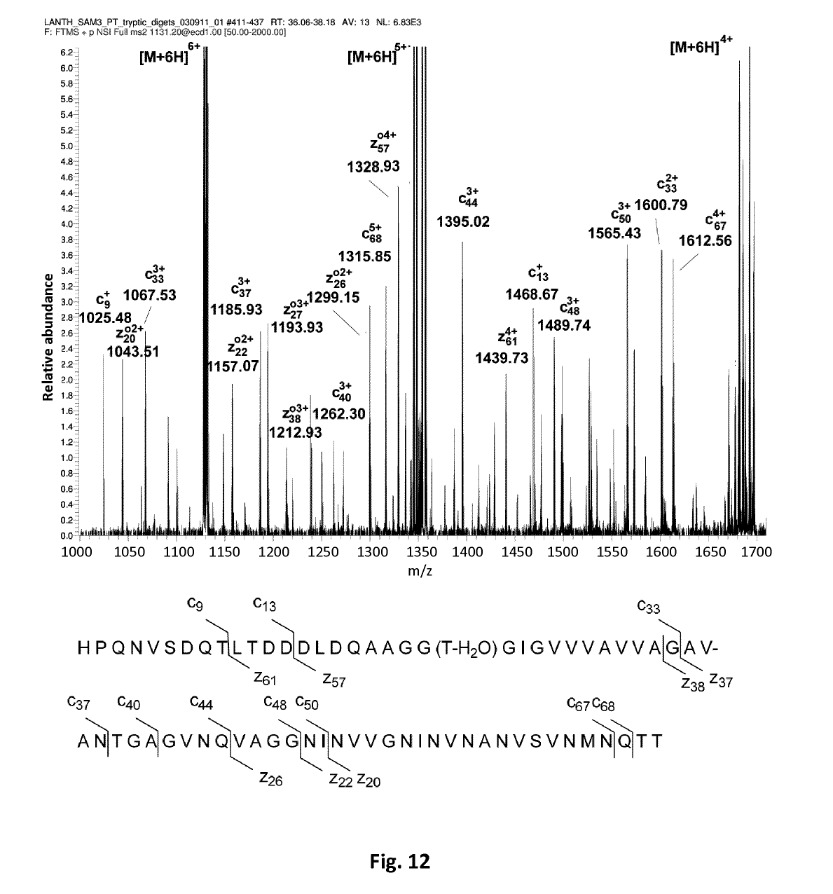

Next, five triple expression strains by adding either poyB, C, E, F, or Ito poyA and poyD were constructed. Unlike all other triple expressions, the strain co-expressing poyF produced PoyA with a mass 18 Da less than expected (observed 17090 Da, calculated 17108 Da; FIGS. 3A, 10A+B and 12), suggesting loss of water and supporting the dehydratase function of PoyF. The modified residue was subsequently identified as Thr-97 by LC/MS/MS (FIGS. 3B, 10A+B and 12).

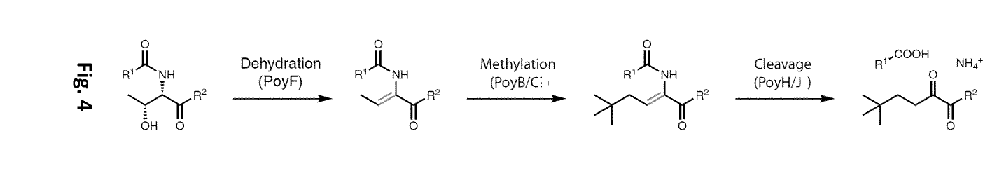

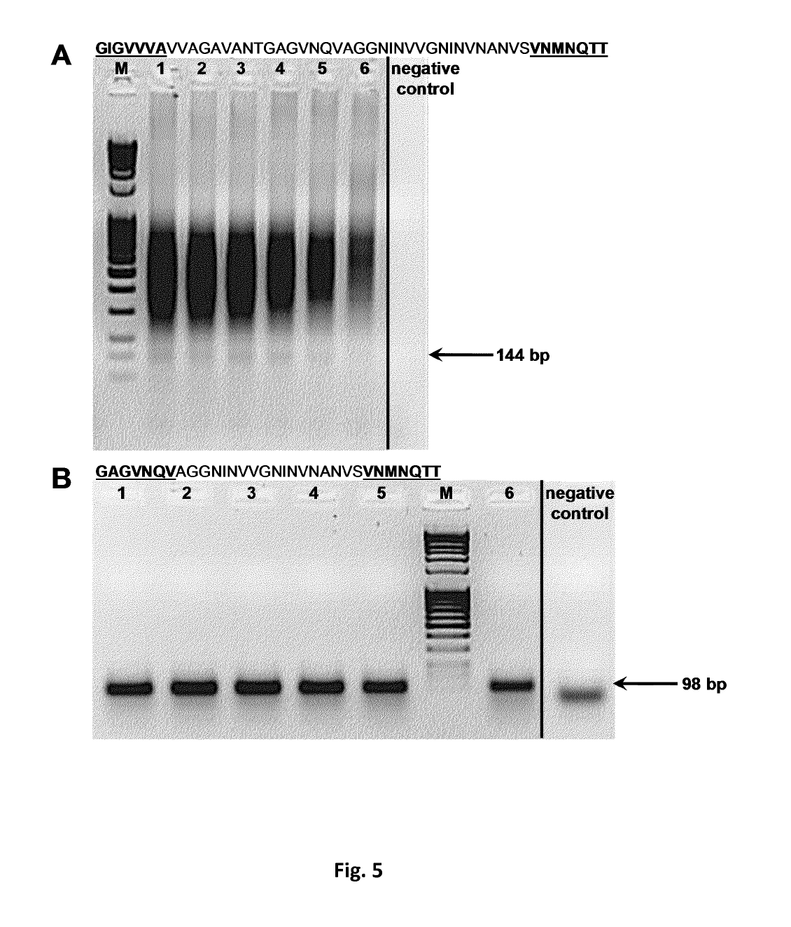

The identification of the Thr residue as part of the PoyA core peptide sequence is corroborated by the presence of an N-terminally adjacent, highly conserved GG motif that was previously proposed as the cleavage site in homologous precursors (Haft et al., BMC Biol. (2010) 8:70). Comparison of the polytheonamide structure with the peptide core suggests that the Thr residue is converted to the unusual N-terminal acyl unit by a remarkable biosynthetic sequence involving PoyF-catalyzed dehydration, formal t-butylation, and spontaneous formation of the 2-oxo moiety (Velasquez et al., Chem. Biol. 18 (2011) 857-867) after hydrolytic cleavage of the enamide (FIG. 4). The inventors are not aware of a precedent for the introduction of a t-butyl group at non-activated carbon positions in biology or synthetic chemistry. This transformation may be accomplished by four successive methylations catalyzed by one or more of the rSAM candidates PoyB and PoyC, both of which contain a cobalamin-binding motif characteristic for rSAM methyltransferases (Sofia et al., Nucleic Acids Res. 29 (2001) 1097-1106). Although radical methylation has been previously observed (Zhang et al., Accounts Chem. Res. 45, (2012) 555-564), this use for extensive modification of peptide structures is notable. In addition, close homologs of poyB and poyC occur in several other ribosomal peptide gene clusters of unknown function (Haft and Basu, J. Bacteriol. 193 (2011) 2745-2755; K. Murphy et al., PLoS One 6, (2011) e20852), suggesting similar modifications in other natural products.

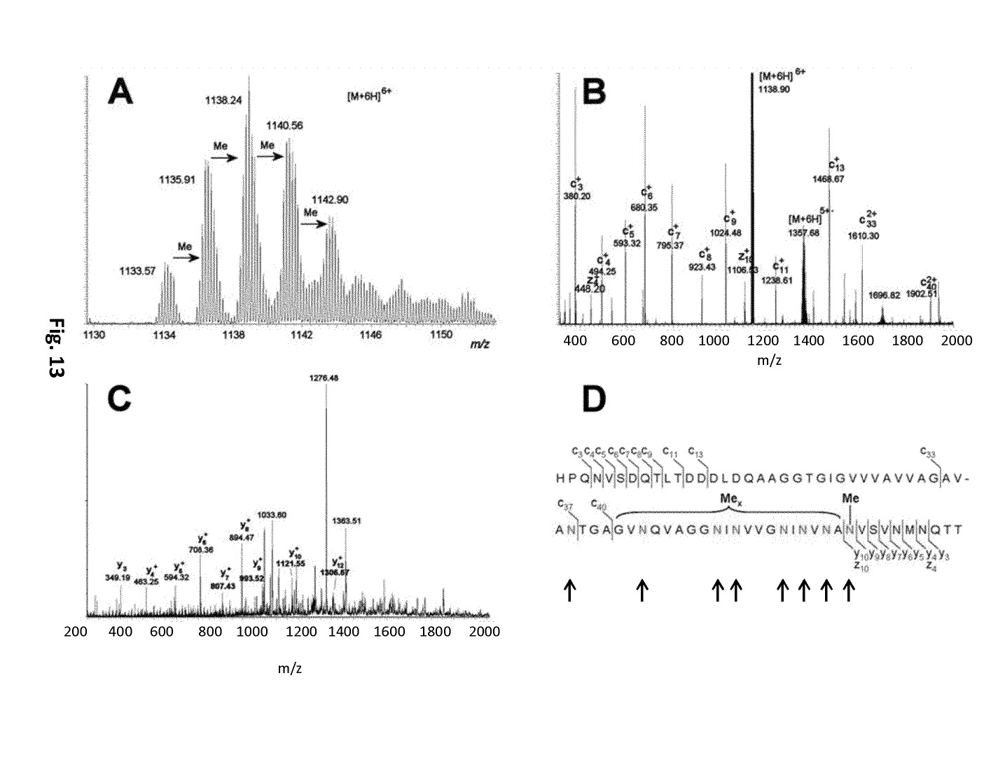

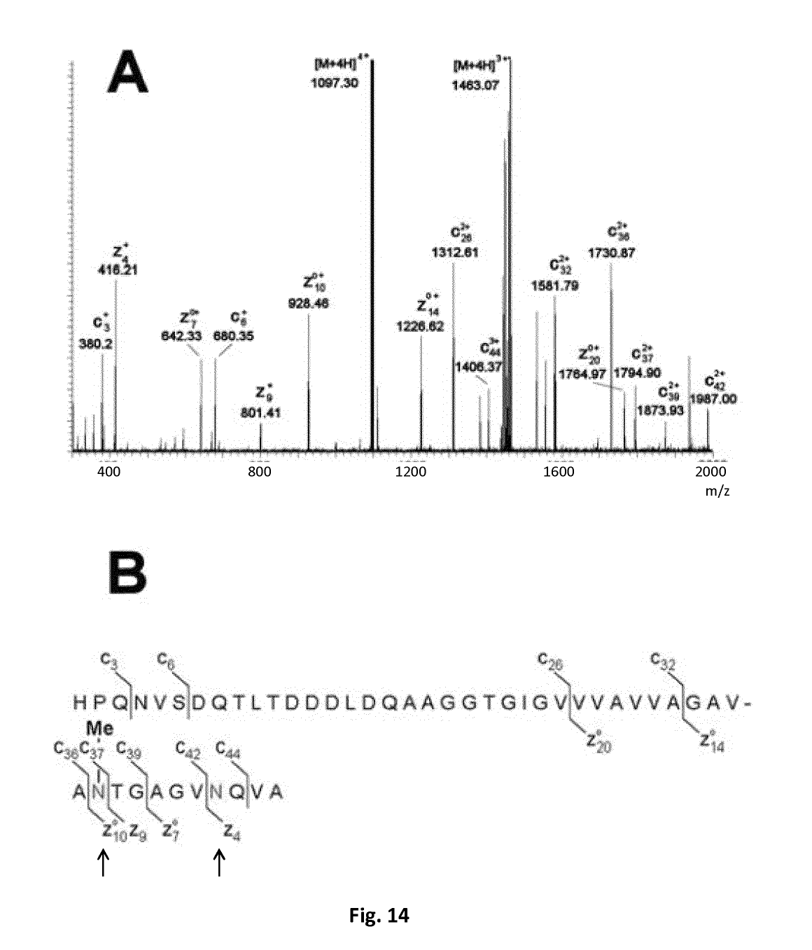

The activity of PoyE was detected with the expression of the codon-optimized poyE (SEQ ID NO: 92) and co-expression harboring two copies of the gene in either 3-day or 7-day inductions at 16° C. In-depth MS analysis of the co-expressed PoyA revealed a suite of peptides increasing by 14 mass units (0 to 8 modifications) correlating perfectly with all expected positions for asparagine N-methylation, indicative of iterative N-methyltransferase activity (FIGS. 13-14). Unlike with NRPS-derived peptides, N-methylation of ribosomal natural products is rare—only N-terminal methylation of the cytotoxin cypemycin has been reported with genetic and biochemical verification (McIntosh et al., Nat. Prod. Rep. 26, (2009) 537-559; Claesen and Bibb, Proc. Natl. Acad. Sci. U.S.A. 107, (2010) 16297-16302). N-methylation of a single Asn has also been observed in cyanobacterial phycobiliproteins; however PoyE bears little sequence homology to these enzymes (Shen et al., J. Bacteriol. 190, (2008) 4808-4817). These data highlight the iterative activities of tailoring enzymes to generate complex natural product architecture. Convincing gene candidates for the remaining two transformation types are present in the poy-cluster suggesting that C-methylation and hydroxylation are also iterative.

TABLE 4
Predicted function of genes of the poy locus and surrounding regions

| | Amino | Proposed | Sequence similarity | Similarity/ | Accession |
|---|---|---|---|---|---|
| Protein | acids | function | (protein, origin) | Identity | number |
| ORF-8 | 448 | Transposase | Tnp, marine | 38/59 | CAC84124 |
| | | | psychrotrophic | | |
| | | | bacterium Mst37 | | |
| ORF-7 | 129 | Transposase | Acid_2946, | 48/62 | YP_824215 |
| | | | Candidatus Solibacter | | |
| | | | usitatus Ellin6076 | | |
| ORF-6 | 49 | Transposition | IstB, gamma | 52/66 | YP_003809448 |
| | | helper protein | proteobacterium | | |
| | | | HdN1 | | |
| ORF-5 | 563 | Unknown | ThimaDRAFT_3015, | 37/51 | ZP_08771276 |
| | | | Thiocapsa marina | | |
| | | | 5811 | | |
| ORF-4 | 139 | Integrase | Acid_1623, | 52/68 | YP_822898 |
| | | | Candidatus Solibacter | | |
| | | | usitatus Ellin6076 | | |
| ORF-3 | 145 | Toxin-antitoxin | CwatDRAFT_0768, | 51/71 | ZP_00518348 |
| | | system: | Crocosphaera watsonii | | |
| | | antitoxin | WH 8501 | | |
| | | component | | | |
| ORF-2 | 70 | Toxin-antitoxin | CY0110_10452, | 58/78 | ZP_01726384 |
| | | system: toxin | Cyanothece sp. | | |
| | | component | CCY0110 | | |
| PoyK | 116 | Unknown | Lbys_1073, | 27/52 | YP_003997151 |
| | | | Leadbetterella byssophila | | |
| | | | DSM | | |
| | | | 17132 | | |
| PoyJ | 578 | Putative | SL003B_2115, | 29/41 | YP_004303842 |
| | | transporter- | Polymorphum gilvum | | |
| | | hydrolase fusion | SL003B-26A1 | | |
| | | protein | | | |
| ORF-1 | 227 | Transposase/ | MettrDRAFT_4459, | 69/81 | ZP_06890742 |
| | | Integrase | Methylosinus | | |
| | | | trichosporium OB3b | | |
| PoyB | 663 | Putative radical | Ava_1133, Anabaena | 41/59 | YP_321652 |
| | | SAM methyl- | variabilis ATCC 29413 | | |
| | | transferase | | | |
| PoyC | 670 | Putative radical | all2023 PCC 7120, | 41/58 | NP_486063 |
| | | SAM methyl- | Nostoc sp. | | |
| | | transferase | | | |
| PoyA | 145 | Precursor | AZL_a09780, | 30/54 | YP_003451053 |
| | | peptide | Azospirillum sp. B510 | | |
| PoyD | 535 | Radical SAM- | Ava_1132, Anabaena | 34/54 | YP_321651 |
| | | dependent | variabilis ATCC | | |
| | | enzyme | | | |
| PoyF | 586 | LanM, N- | Npun_R3205, Nostoc | 30/45 | YP_001866601 |
| | | terminal | punctiforme PCC | | |
| | | dehydratase | 73102 | | |
| | | domain | | | |
| PoyG | 110 | Chagasin-like | MCAG_02545, | 44/50 | ZP_04606288 |
| | | peptidase | Micromonospora sp. | | |
| | | inhibitor | ATCC 39149 | | |
| PoyH | 323 | C1 peptidase | MCAG_02544, | 34/50 | ZP_04606287 |
| | | | Micromonospora sp. | | |
| | | | ATCC 39149 | | |
| PoyI | 390 | Fe(II)/α- | MXAN_4411, | 35/52 | YP_632582 |
| | | ketoglutarate- | Myxococcus xanthus, DK | | |
| | | dependent | 1622 | | |
| | | oxygenase | | | |
| ORF1 | 227 | Integrase | MettrDRAFT_4459, | 69/81 | ZP_06890742 |
| | | | Methylosinus | | |
| | | | trichosporium OB3b | | |
| ORF2 | 63 | Integrase | CLOSS21_03037, | 60/75 | ZP_02440531 |
| | | | Clostridium sp. SS2/1 | | |
| ORF3 | 174 | Transposase | all7004, Nostoc sp., PCC | 31/47 | NP_490110 |
| | | | 7120 | | |
| ORF4 | 57 | Unknown | no homology | | |
| ORF5 | 292 | Integrase | Hypothetical protein, | 46/64 | ACB12942 |
| | | | Thauera sp. E7 | | |
| ORF6 | 412 | Transposase | Bphy_6606, | 45/60 | YP_001862680 |
| | | | Burkholderia phymatum | | |
| | | | STM815 | | |
| ORF7 | 43 | Unknown | no homology | | |
| ORF8 | 791 | Transacylase | Bcere0004_56000, | 49/69 | ZP_04315177 |
| | | | Bacillus cereus BGSC6E1 | | |
| ORF9 | 505 | Reverse | Bcep1808_7013, | 58/74 | YP_001110702 |
| | | transcriptase | Burkholderia | | |
| | | | vietnamiensis G4 | | |
| ORF10 | 149 | Unknown | no homology | | |
| ORF11 | 243 | Phosphopantetheinyl | Hypothetical protein, | 73/80 | AAY00051 |
| | | transferase | uncultured bacterial | | |
| | | | symbiont of Discodermia | | |
| | | | dissoluta | | |
| ORF12 | 698 | Cation | CtpC, Beggiatoa sp. PS | 46/68 | ZP_02000412 |
| | | transporter | | | |
| ORF13 | 40 | Transposase | Nwat_1386, | 79/94 | YP_003760623 |
| | | | Nitrosococcus | | |
| | | | watsonii C-113 | | |
| ORF14 | 53 | Transposase | Nwat_1386, | 57/80 | YP_003760623 |
| | | | Nitrosococcus | | |
| | | | watsonii C-113 | | |
| ORF15 | 45 | Unknown | no homology | | |
| ORF16 | 563 | Unknown | ThimaDRAFT_3015, | 37/51 | ZP_08771276 |
| | | | Thiocapsa marina | | |
| | | | 5811 | | |
| ORF17 | 52 | Unknown | no homology | | |
| ORF18 | 306 | Unknown | Amet_0218, Alkaliphilus | 24/50 | YP_001318112 |
| | | | metalliredigens | | |
| | | | QYMF | | |
| ORF19 | 50 | Unknown | no homology | | |
| ORF20 | 66 | Unknown | Franean1_3030, | 70/82 | YP_001507349 |
| | | | Frankia sp. EAN1pec | | |
| ORF21 | 43 | Unknown | no homology | | |
| ORF22 | 458 | Transposase | Cyan7822_6238, | 50/65 | YP_003900107 |
| | | | Cyanothece sp. PCC | | |
| | | | 7822 | | |
| ORF23 | 146 | Transcriptional | Rcas_3701, Roseiflexus | 46/62 | YP_001433759 |
| | | regulator | castenholzii DSM | | |
| | | | 13941 | | |

### Example 13

### Antibacterial Activity Testing

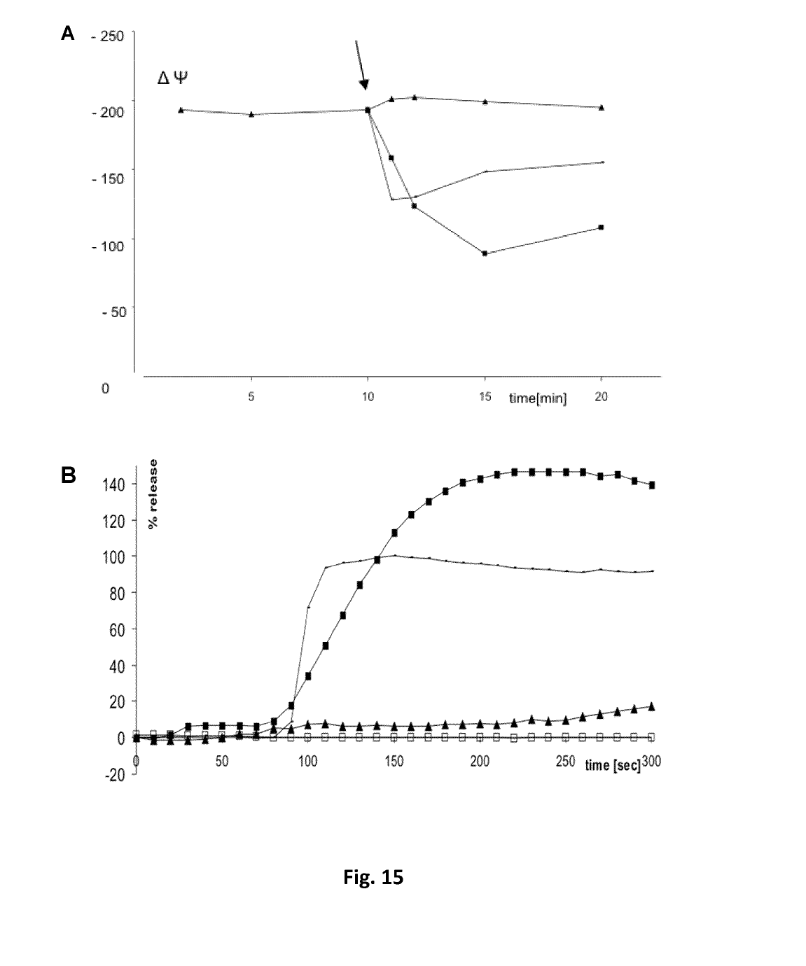

Mature polytheonamides form minimalistic unimolecular ion channels in cell membranes (21). This mode of action was further investigated herein to show possible antibacterial effects in more detail. Minimal growth inhibitory concentrations (MIC) were determined by the standard broth microdilution method based on the most recent Clinical and Laboratory Standards Institute guidelines (see Table 5 below). The membrane potential was estimated using the isotope-labeled tetraphosphonium cation (TPP⁺); its intra- and extracellular concentrations were applied to the Nernst equation (see FIG. 15A). The release of intracellular K⁺ ions was followed online using a potassium electrode; exemplary shown in FIG. 15B for Arthrobacter crystallopoietes DSM 20117. Microbiological assays were performed as described in the Materials and Methods section on pages 1611-1612 of Schneider et al., Antimicrob. Agents Chemother. 53 (2009), 1610-1618, disclosure content of which is incorporated hereby by reference.

TABLE 5
Minimal growth inhibitory concentrations (MIC) of
polytheonamide B on exemplary Gram-positive bacteria.

| | MIC (μg/ml) |
|---|---|
| organism | polytheonamide B |
| Enterococcus faecium spec. | >125 |
| Micrococcus luteus ATCC 4698 | 8 |
| Bacillus megaterium spec. | 8 |
| Arthrobacter crystallopoietes DSM 20117 | 4 |

As may be seen in Table 5 above, polytheonamide B has been found to be active against Gram-positive bacteria with minimal inhibitory concentrations in the μg/ml range of concentrations. The peptide rapidly depolarized the bacterial cytoplasmic membrane, simultaneously decreasing the membrane potential and intracellular K+ contents, which is consistent with the formation of transmembrane ion channels (FIG. 15A).

In summary, the experiments herein provide evidence for a bacterial origin of a sponge-derived peptide natural product. While polytheonamides are currently the only attributed proteusin members, a small number of compounds exhibit structures that suggest a close biosynthetic relationship. These are the sponge-isolated yaku'amides (Ueoka et al., J. Am. Chem. Soc. 132 (2010), 17692-17694) and discodermins (Matsunaga et al., Tetrahedron Lett. 25, (1984) 5165-5168), all of which contain residues with additional C-methyl groups and D-configured α-carbon atoms. The use of ribosomal machinery to generate products containing D-amino acids and other modifications offers a new possibility for the artificial engineering of peptides, peptidomimetics, and proteins with new structural and functional properties.

1. A nucleic acid molecule or a composition of nucleic acid molecules comprising:
(a) a nucleotide sequence of SEQ ID NO: 1;
(b) a nucleotide sequence capable of hybridizing to the nucleotide sequence of SEQ ID NO: 1 under stringent hybridization conditions and encoding at least one polypeptide which catalyzes at least one step of the biosynthesis of polytheonamides and/or encoding a precursor peptide thereof;
(c) a nucleotide sequence encoding at least one polypeptide or peptide selected from any one of poyA (SEQ ID NO: 3), poyB (SEQ ID NO: 5), poyC (SEQ ID NO: 7), poyD (SEQ ID NO: 9), poyE (SEQ ID NO: 11), poyF (SEQ ID NO: 13), poyG (SEQ ID NO: 15), poyH (SEQ ID NO: 17), poyI (SEQ ID NO: 19), poyK (SEQ ID NO: 21) and/or poyJ (SEQ ID NO: 23);
(d) a nucleotide sequence encoding at least one polypeptide which catalyzes at least one step of the biosynthesis of polytheonamides and/or encoding a precursor peptide thereof which amino acid sequence is modified compared to the amino acid sequence of any one of poyA (SEQ ID NO: 3), poyB (SEQ ID NO: 5), poyC (SEQ ID NO: 7), poyD (SEQ ID NO: 9), poyE (SEQ ID NO: 11), poyF (SEQ ID NO: 13), poyG (SEQ ID NO: 15), poyH (SEQ ID NO: 17), poyI (SEQ ID NO: 19), poyK (SEQ ID NO: 21) and/or poyJ (SEQ ID NO: 23) by way of one or more amino acid substitution(s), deletion(s) and/or insertion(s);
(e) a variant or portion of a nucleotide sequence of any one of (a) to (d) encoding at least one polypeptide which catalyzes at least one step of the biosynthesis of polytheonamides and/or encoding a precursor peptide thereof;
(f) a nucleotide sequence which is degenerated with respected to the nucleotide sequence of any one of (a) to (e); or
(g) a nucleotide sequence which is complementary to the nucleotide sequence in any one of (a) to (f).

2. The nucleic acid molecule or composition of claim 1, wherein the nucleotide sequence(s) comprise(s) at least the coding region for any one of poyA (SEQ ID NO: 2 or 90), poyB (SEQ ID NO: 4), poyC (SEQ ID NO: 6), poyD (SEQ ID NO: 8), poyE (SEQ ID NO: 10 or 92), poyF (SEQ ID NO: 12), poyG (SEQ ID NO: 14), poyH (SEQ ID NO: 16), poyI (SEQ ID NO: 18), poyK (SEQ ID NO: 20) and/or poy J (SEQ ID NO: 22), including variants or portions thereof, wherein the variants or portions encode a polypeptide which retains the biological activity of the respective polypeptide.

3. The nucleic acid molecule of claim 1, wherein
(a) the encoded polypeptide and/or peptide differs in at least one amino acid from the amino acid sequence of SEQ ID NOs: 3, 5, 7, 9, 11, 13, 15, 17, 19, 21 or 23; and/or
(b) the nucleotide sequence differs in at least one nucleotide from the nucleotide sequence of SEQ ID NOs: 1, 2, 4, 6, 8, 10, 12, 14, 16, 18, 20, 22, 90 or 92.

4. The nucleic acid molecule of claim 1, wherein the polypeptide and/or peptide encoding nucleotide sequences are operatively linked to at least one expression control sequence, preferably wherein at least one expression control sequence is foreign to the polypeptide and/or peptide encoding nucleotide sequences.

5. A nucleic acid molecule capable of specifically hybridizing to a nucleic acid molecule of claim 1 under stringent hybridization conditions.

6. A pair of nucleic acid molecules which correspond to the 5″ and reverse complement of the 3″ end of a nucleotide sequence of the nucleic acid molecule of claim 1.

7. A vector comprising the nucleic acid molecule of claim 1.

8. A host cell, which is preferably a microorganism, more preferably which is a bacterial host, comprising the vector of claim 7.

9. A method for preparing at least one polypeptide which catalyzes at least one step of the biosynthesis of polytheonamides and/or for preparing a precursor peptide thereof, said method comprising:
(a) culturing the host cell of claim 8 under conditions allowing the expression of the nucleic acid molecule; and optionally
(b) recovering the polypeptide(s) and/or the precursor peptide.

10. A composition comprising at least one polypeptide which catalyzes at least one step of the biosynthesis of polytheonamides encoded by the nucleic acid molecule of claim 1, preferably which is a kit or a diagnostic composition.

11. A method for preparing a selected peptide-based compound or precursor thereof, preferably wherein the peptide-based compound is a polytheonamide, and/or for the manufacture of a medicament for the treatment of a tumor, said method comprising:
(a) culturing the host cell of claim 8 under conditions under which the cell will produce the polypeptide(s) and a precursor peptide; and
(b) recovering the peptide-based compound;
preferably wherein the cell does not produce the peptide-based compound in the absence of the nucleic acid molecule.

12. A peptide-based compound obtainable by the method of claim 11.

13. The peptide-based compound of claim 12, which is a polytheonamide.

14. An antibody specifically recognizing a polypeptide or peptide precursor encoded by the nucleic acid molecule of claim 1.

15. An agent comprising a peptide-based compound produced by the method of claim 11 which is covalently or non-covalently linked to a functional moiety, preferably wherein the functional moiety comprises an antibody or an antigen-binding fragment thereof, further preferably wherein the antigen is a tumor antigen.

16. (canceled)

17. The host cell of claim 8, further comprising a gene encoding a selected precursor peptide or protein, which is not encoded by a nucleic acid molecule of claim 1.

18. An antibody specifically recognizing a peptide-based compound produced by the method of claim 11.


SEQUENCE LISTING (93 sequences)

SEQ ID NO: 1 - DNA, 16366 bp, Artificial Sequence
  poy gene cluster
ctataaatccgcacggctcaggccaagttgggcgagcatggcatagagcgtccccgttttcaattcatctctgggattgcgcacaatggtgagccgttcgccatagtacaaggtcccgtggcttcccttaccacgctcggggacaaattcgaccctgacgccgcgttgacgtccaagccttcttagtcgtcgaatgaattcgttgccagtcatagaagggagtatcggacatatatgaccgatgatcaagagaaatgtgcagcctattccgatggccgtcatcaaatagagcgattcgaatgcgaaggggcgttaatggggtcgtatgagttttttggccacacaggtggcaactttgccgtggaatcgatggcaactttcgcgtggaatatgcagcagtgactccgagtctatcgtggacgatagaggcgctgtttgttaggataggccactcggctcaaaagtggtgacaaaaccggacgacacccaggtgagccacccatctttcaagcctctacccaagaggagttgttgcaatgcttcagacccaatgtttgaaagtaccaatcatcccaggcaaagaggctgaggtcaaaaattggctcgccgcgctttcgactcgacaccaggaggtgctggaagcgatcacatcggaagacatcgctgatgaggccatgttttacgcgaaagaaccatccggcgagttcctctacttatactctcgtgcaccggacttggcagtagctggcgccgcgtttcagaaatcccaattgcccatcgatcaagagttcaagcgaatctccgcggaatgtctggactaccgttcagccatccgacttgaactccttctttctgcggatagccgcaacaagtatgtgtatccctaaatcggtccacctgagattggtacagtgaggttctctatgcgacgttggattttcatgctgatccacgtttaaggagaactcaccgtgcctgcagcttcacttgcaccggcgctcaggacgcgtgttcgctcagccaggctgcgacgcgctggaggaactccggatcgacgtggcgggtttgatcgaagtactgggccattgtcgattccccagctgtcgccttgaagaggtgatcgagattggggaagagcgcatacgtacaatcaccgcccgcactttgagctgcggtaacgagcagctcggcatcgcgctcggcggagacttggaagtctttgctaccctgacacacgagtagtgggcactggagccgggtaatgagttccactgtccggtataacaataactgtttcagccaagtcgggctgcggaacatcgcgaggaggtagtgggggatattgtccggattccacggatgatctgccttcaccaactctaccgcctcctgaacctcctgaatctgctgctcgatttgctcgtccgtcaagccgatgtctttccccatgtagacaatctggtcggtgatgacgtcctcgatgctccggcccatggtggccatcaagaccactccgcgcagggcaataccctcttgctcggcgagcaccagagccacagtgccgccctggctatggccaaggaggaaaacggcacctccagccgcttccgggcgagcgcgcagaaattctaggcatgcccgggcatcagcaatttccgaatcgaggccgcgaaccagagtgtcctcgcccattttcgtcgtgcccgccccgcgggaatcgaaacgcaaccccagaaggccttgctccgccaaaaagtccataatttcgtgcgttcccatatctatttcacccgcgatcccgtgacgatcatgagtgcccgaaccactcaggaagagcacggctgggaaggggcccggaccttccggaatggagagcgtcgcaccgattggcgtcaccgggccggggatagtgacgtcctcaagacggaagcgggtattgtcgcgatagtggtagcggagcgggacggtgaccggctgatcctcgtcaagccattgcggcactggcggaccagggcgctccagccatccgcggaccccttgcgtcggcaccatgccttcgatcatcaacccgtgttcatcgaggtgaatctcttcaagatgcgaggagcggtgccacgtgccggactcggcggcaaggtcgagtgcgggactcatctggtagggaacggccgccagctgattggcgagaaagagtccaactgtcacctcttggtcgaggagcttgcggcatgccaaggcggcaaagatcagtgccttgtgccccggcacattgtcggcggcgatgaactgcgcctccccgcgcggcacgacttgctccgatccatcgggaagcgtggccgtcacctcggccgattcaaagcggacctcccagcgagtaccacctgcctcgctgaggtagtgcacgggatcgagtgcgccgttgcagagcaagacgctgcgttgcctcacaagttctccccctagacccatcgccaccggatcaagctcaaggaagaattcaatctcgtggagatcaccctcgcgtggcccccggtacacggtccactgcgtcgcatagcgctcatcggcaatcgacaatgcatagacgagcggctcgccgttgggaaacaaaacggcaacccagccaggagccacctcttgatccgcagaagcgtccccggcgggagcggcactggagtcggtaccggcttcagtggcgtccgctggcgtcgtacccattctattggattggctcatggcttacctcctcgcccggagatggtgattgattagctgccattatcctatcaacgcaattatagggtcgatgcaccagcctgtccatcaggggcaattcaagcggttccgtcgcaaattttcatctgaagccatttggggtatgatctccctctataatgagcgatgcatgaggagggagacagacgatggcgatcagcttcaaaggtgcccattttccacccaaagtcattttgatgggagtccgatggtatctggcgtaccctttgagcacgcgccacgttgaggaactcatggcagaacgcggggtccacgtcgatcattccaccgtcaaccgctgggtggtgaagtacagtccgcagctcgaagccgaattccaccggcgcaaacgtgcggtgtggacgagttggcggatggatgagacgtacattaaagtgaaaggcgagtggaaatacctctaccgggctgttgacaaattcggcaagaccattgattttctgctcacggagcagcgtgatgagaaagcggccaggaagtttctcaacaaggcgattggccgccacggcagtgtgccggagaagatcacgatcgatggtagtgcggccaacgaagccgccatcaagagctacaacaaggatcacggcatgtcaatcgaaattcgccaggtcaagtatcttaacaacatcgtggaacaggatcatcgaggtgtgaagcggattacgcggccgatgctggggttccaatcctttgactccgctcagtccactctcactggcatcgagctgatgcacatgctgcgaaaagggcagcttgaagataggggtgagcagggactcagtgtggccgatcagttctacgctctggcctcgtagtcaccctaccaaacgaggttcactcaccccaaagcagcgagacacccatatttgcgacggaacctcactactttccagctcgggtgaaagatcccaattattcttaaaaagtcgcttgacaaagtgcatgtgttagttcagaattccccaatgttcaaactttgacagcataaactcgtagtacctttcccatcagcgaacacagccgacgataactgattctgcttgttaggagataggatcatgagtgatgtgcttctagtctcggtaccgtacgcagccttgcagcacccatcccctgcattgggctgtctgcaggccgtgctccggcgccgtgggatcgaagtccacaccatgcatgcgaatctccggttcgctgaacggatcgggatcgggaactatacatggttcggtacctacagccggccacagttgctcggcgagttgacatttgccaaggctgcttttcccgacttcgagcccgatcttcacgcctatgcccaggtcattaacgtaccggaagcggaacatgctatccgcgacgtgcggcaagccgcggtttcgttcatcgacgagatggctcagcagatcatcgagcaagatccgaagattgtgggatgttcctcaacgttccagcagaattgcgcctcgttggcgctcttgcgccggctccgcgagctgtcaccgggcatcgtcaccgtcatgggtggagcaaattgcgaaagcgagatggggcgggctctccatacgaacttcgactgggtagattacgtcgtctcgggtgatggggacgaggtgttccccgacctctgcgagcggattcttcacaacgacattgcgggcatcgcgtctaacgaggcgctgcgccgtttcgtattcacccccgcgtcccgaccgttcgctaattttcaggtggtcgagcgcgccacgactcaggacatggacggattacccctgccggactacgacgattacttccgcgaattggcagagacaaaaatcgaatactttgcgacacctgggcttctcgtcgagaccgcgcgcggttgctggtggggggaaaaacacccgtgcactttttgcggcctcaacggtgggtgcatgagctttcgggcgatgagcccggagaaggcggagtggcacatctgcgaattgtccgcccgctacggcatcgacgggatcgaagtgattgataatatcctcgcgccttcttattttaacaccgttttgccggccctcgcccgaaaggaaaagcgcttgcgcctagcctgcgaggtaaaggccaacctgaagcgggagcaagtgaaagcgctggcggacgcgggggcaatctgggtgcagccgggcatcgaggccctgcacgatgaaacgcttaagctcatcgacaagggcgcgacggtctgccagaacctccagttgctgaaatgggctcgagaatacggcgtccatattacctggaactatctacttggcattccgggcgaacggagggcgttatacaaggaggtcgcggacctcttgcccctcatcatgcatttacaggcacctaatggaccgggctcgcggctcagctttgatcggttcagcgtctatcacacgcatgcagaccattatcagctcacgttgcgtccggcatgggggtattcgaacgtctacccattgccgatcagccagctcatagatctggcctatcacttcgacgatgtcggtcctaacgcggtgctgcgctccgattttccggaaatggaactcttacgagagcgtttgaaggaatggtgcgatctgcagccggagccttccggctatgatgacccggcggatttgcctcccctccccaggctagatgtctttgaacgagacgacggcagactcctcatcgaagacacgcggccgtgcgctccggcgacgcagcgcgagctctgtgggctcagcgcctatctctacaccgcgtgcgaccgaggccgtactgcagcacggcttcggtccgcttgtcacgacgagggcttctctgaggccactgcggccgacgtcgatcgcgagttgacacgcctcattgacgacaagctcatggcttttgtgagcaaccggtttttgagcctcgctgtgcgggcgccataccccgcctgccgacccctgagtgaacaccccgacgggcaggtgtatctacggcccgtccagaagcaccgcgagccgcgcgagcagacgattcaagacgtattcgggcttaagctctgactgggcgggatgggcttcggaaactcttggaaaggtattgtatgccaacaaacgatgtggtcctgctcaacatgccgtattcagccatcgagcacccgtcgatctccctcggatacttctcggccagcctgaagcagcgcggcataagtgtcgacacgatctccgctaatgcgttcttcgcgcgtgatatcgggctcaaggagtactttctcttcagcaactattacaacaacgatttgctcggcgaatggacgttctcgggggaggcattcccggattttcaccccgatcacgacacctatttccgcgatcttcaacttccgattccggaggcgaagatccgtcgcattcgcgagctcgcggcaggtttcatcgaccagatgacagagcgggtcttgtctcagaacccacgcatcgtcggctgcagctcaacctttcagcaaaactgcgcctcgctcgccctgctgcgccgggtgcgggagcaagcgccggaggtcattacagtcatgggcggtgcgaactgtgaggggccaatgggtaaggcgctcaagcagtgtttcgactgggtggatcacgtgttctcaggcgaggcggacgacctttttccggagttctgtgcattgatcctggacgcaactgaccgggagcaagccatgcgccgcatcgctgactgggcgcctgggtcgatctttcaggtctcaacagggctcatgctgcaggcggaagcgagcgagcgctctgtggctaaggatctcagttctctcccaacccccgattacgacgactacttccgcgaccttcaggaagccggggtcgcccggcaggttctgccggggctcatgcttgagacctcgcggggctgctggtggggagagaaagacgtctgtaccttctgtgggttgaatggcgaatacattaacttccgagccaaagaccccgacgtggtgcaccgggagctccgcgatctcacggcccgctacggcatcaatgcgttcgaagtcgtagacaatatcctgtctatgaaatactttaaaacgctcttgccgcagattatcgaatccggagaaaagtatgggttcctttatgaaatcaaggcgaacctgagacgcgatcacgtggagatgctggccgccgctggcgtgctttgggtacaaccgggtatcgagagcctggatgatggggtactccggctgattcacaaaggtgccacggcctgccagaacttgcaactcctcaagtggtgccgcgaatacgggctctttgtcatctggaactacctgtgcgatatccccggtgaacatgacgagtggcatgccgacctggtcgacctcctgccgcaaatcgttcattttcagccgccttcgtcgccgggatcacccttgcgttttgaccgcttcagcgtctatcacaattatccggatgagcatggcctggagctgacccccgcgtggacctactcctatatctatccggtcagcgacgcacacatccagcggatcgcctatttcttcgacaatcaccgcccagatgcggtcaacgatggaaagaaccgcccgctctgggagcaggcgcgtgaggagctcgtggagtggcgcaatctccattaccagtatcaggaagatgacgtgtgggcggaggtggcgtccggctcgccgatgctaagcatgtcatatggcgacggggatctattgatccttgaagacacgcgtccctgcgccgttcaatcgcgaatcgagctccgaggtgccgcggcccgaatttacgagctctgcgacgaaggtcgcaaggcatccaccgtgctgtcgcactgtcggtcgtccggttgtccggatctcgacgcggctgaggtcgaccggattctccaaacattcctagatcacaagattatggcctttgtcagcgaccggtatttgagtcttgcggtgcgtgcgccgtgggcctcgtacccatcgatggagttgtttccaggcgggcgcgttctgctcaagcgcgctgcggctccgaagaaaccccaagaactcacagtcgccgacgtgttcggggtaaaagtctagatttccattctaacccagaaaggagtccaccatggcagacagcgacaatacgcccacatcgaggaaggattttgaaactgcgatcattgcgaaggcctggaaagatccggaatacttgcggcgcctgcggagcaatccgcgtgaagtgctgcaggaagaacttgaggcccttcaccctggggcgcaactccccgacgatctcggaatctcgatccacgaggaagatgaaaatcacgtccatctggtcatgccgcgccatccgcaaaacgtgtcagaccagaccctgacagacgatgacctcgatcaggccgcaggtggcaccggaatcggcgttgtggtggccgtggtcgccggtgccgtcgccaacaccggcgcgggtgtcaatcaggtcgcaggtggcaatatcaatgtggtggggaacatcaacgtcaatgccaacgttagcgtgaatatgaaccagaccacataatgcctgccccgaaagtacatcccccgcggcgttcggctggggacgtattgctcgtgaagaggctctcaggaggccaagatgaacctgcagtcgatcgactctcaggcagttcgttcgaaggtggatgcggaatacccgtcggctgcgcatgtcaagcgatttctggaggtttggtgctcagggctctataagaaagaagacttgctcacccgaccgcaggaaatcctcgatgctcaccacgtagcgatagatccgtccctcatctccgttctatttgaatcgaaattcttgcgggggaaatccggcaagtttgacctgctaccgccgcagttcgacgcgtttcgcgactttatgatgacgaagatccagtggcggcaacaaatccgcactggctctgctcctgccgaccccatatttcgtgaatttcgggagcgtcagatccagagatgtgagatcgaactcggcagcgatcagaacaccgccatcgttcacacgcccgtcgtgttcgagctgacgcggggctgctcggtgaagtgttggttttgtgcgctcgacgcaccgcctctcacggggatctttgactattccccggaaaacgcacgatttttccgggacgtgcttcgcgtcgtcaaagacgtgattggtccagcgagcaaatgggccagcagctactggggaaccgacccgctcgacaaccccgatcaagagaaatttagccttgatttccgtgaaatcctcggcatgtatccgcagacgacgacggcgatcccactgcgcgcgcccgagcgcacgcggaagttgcttgaggtgtctcatgccagtggctgcctcgtcaaccgcttctcggttcgtacaccagcgcagctcagaaagattcacgatacgttttcggcagaggagctgctctacaccgagttggtcttgcagaatctggaatcggacagtgtgaaggcgcgtgcgggccgcttgatccatttcgccgatgatctccccaaactcgctgcgaaagatgaggaaaagctgctcaatatgctccaagagcgagaacccgagctagccgcaaaagcaacgtcgattctcattaatctcccaggctccaccgaaccgatcatcagggccacgagtaacgccgatgaagaggatacctccgaggaatacaatgtgtccatcaatgtcccgggcactacgtcctgcctgaccggcttcaagatcaacatggtcgatcgcactgtggagctgttgagcccctgtccagcaaacgagcgatggccgctcggacacattgtgtttgaggaagggacgttcgacacagccgaagacctgaggacgctcatgctcggcatgatctctcgcaatatggctgaacgggtcgttcccgagtcgttggtgcgctttgtcccgcggctcatctatcgcgaagacccggaggggttccgccttggttccgtgttcggcaacggtgtgatctgtcatgacccgactcgatcggcgtacctccatcgtcttggcaacctgctccgcgaggggaaaaagcgagccggcgagatcgcgatgctgtgcttttatgagttcggcgtacctgaaaattacacgatgggaagcatcaacaacatgttccatcagggcatcatcgcggaggagccgatcgccacagaggcgccgatcgccgtggcggcttaccagtaatgacgccgaggtcgcttccgaccgagccgctcgcggcccctgccgccgcggctctcgatgccgcacttcgactgctcaaggaatacctggacgatatcggatatcacggcatctacaaatacctggccacggcgaactattatgccgcttcgccctccgtgttcaatgcgtctcgtccccaaagccttgaggcgttcgatcggcaaatccacgagggaccggatgactggatgctcgccagatgcctcacgacgtgcgcgccgtgccgtctggaagctctgccgcccccggcgcgaagggtggccgaggtcctggccgatgtcggcctcctcgtgtggaacgggaacacgcttgagcaaggaggttatcagctcatctctgtgttcgatcgttatattttactcgatgcgcggattcacttcggcggcagtcagctccacgatgtctatattggtcccgacagccacctcttgctgtattacatgccagtggaagcgatcaggccggcagaccacatcctcgacctatgcaccggcaccggggtgatcggacttggcctgtctcgattctccgagcatgtcgtctcaactgacatcgccccgcctgccctgcgtctagcgcatatgaatcgggcactcaacaacgctgaggggcgcgtgtcaattcgcgccgagaatctgcaagagacactcgctagcgatgagtgtttcgatctgatcgcgtgcaatccgccatatgtcgccgcacctcccgagcttcccacgccgctctatgcgcagggtccggatcgggacggcctaggctatctgcgcctgctgatggagcgggctcctgagaagctcaatccgggtgggcaggcgatgtttgtcgtcgatctcattggcgacacgcatcgcccctactacttcgacgacttggagcgcatcgcgaaggagcaagagctgtttatcgaagccttcatcgataaccgtttgaaggcggacgggcagctcccggcttacaagttcctctacgcgcggctgtttcatggcactccccctgaagagatcgaacagcgcatgaggaactttattttcgatgagctgcacgcgtactactattacatgaccacgctccgcgtgcggcgccgcaaaccatccggtttgcgtgtactcgaccggtacaggatcaccagctatgatgagttcttccagcagtcgtgagggagacgccctcgcccatgctgctgatcccacaaggtcaattgcggacgacatcacttgtgggctcgactcgctaggcggccctcccacggtatgcgatgctgaacatccggtcccatttgaggagctatggctcgggttcgtcgcgcacgcggctcgtgcgcttgaaccgctcatcgcgccgtcgttgcacgcggctgtgttacacgatccgcacggacccctgcgctcgctgctcatcgagctggccgagatgggcgcgccagcggctttcgagttgtttgcactctatcgcatccgggaggcgagtgccgcgtccgtcttcggaagtgtccgcggggtagagagtcgagacacctataacgcattcgtgaagcacctcgccactcagcagctctccccgctgtgggatgcctttccggccctcaagcctctgctggccacccgtaccaatctctttatagccgccatggcagagttgtgccagcggtgggaggccgatcacgtagagatcatgtcggtcttcccggagttacgagggataggtgcaccgcagcgcattcgccccggcttatccgatgcgcatggcggtgggcgaaccgttactcgcctgtcattcgccggtggtgaggcgcttttctacaagccacgcccggtcgacatggagtggggttgtgcacagtttgtcgagtggttcaacggccaagaccacgggatgccgccgctccgggcgctttcggtcttgcccagaaccgactatggctggatggaggcggcacgccccgcactgtgcacacatgttgacgcagtcgcccgattctaccaccgcgcagggatgctgctggccctcgccgaccttttctgcggggtcgatttccatagtgaaaacctgattgcaagcggtgagtatccggtaatcgtggatctggagaccttgtttcatccgctggggcctttcgaatcgcagggcgatgccttggagcgcacagagctgctgccacggcccatctactgtgaagatggtgccccctatgtcatctgcgggttaggtgtggttcctggaaaggcggtcattgagctccggcggcgcggatggatcaacatcaattgcgataacatggcgtcctgtgacgtgaccgtcccttggcctgtcggtggcgccgtgcctcgcaacaaagagggtgcggcgccgtcgatcgcgacatggtcgggcgagatcgtctcggggttctctgctatgcatgtgttcttccgcacacgccggaacgagctgtggagcgccgacgggcctatcgtgcaagcgtttggcggcgggcgttcgcggtttctcctacgcgcgacgcggatctatgtggagctactgcgccgggccgtgcagccgcaagcgctcgcagctcacgcgtcatcgagccacatcttcgatcgtctggcgcgcacgggccggggatgggatgagatccatcgcgtggagcggcaggcgctcgatcgcatggacgtgccttatttcaccatggaaaccaccgcgcagtttgttcgcggcgagggccgtaccatccatgcattcttcgccacgtccgggcttcacatggcacggcgccggtcggagttcatagacgatgtcattctaaatgacagggccgcgtcgattcgcaatgttctcgaacaatcgagttacagcggggaggcgaccctaccggactccagagtccagaccgacgctggcgcaggagcctgagctatggataacgcgacacaccatttcgaacagagccattttggcaccttacacgaagtggggctatacgacgaagtgagtattgagcttcttgagtacgctacggccggctttaggtgggagatcacttacgagtcgcccgaagcagtcgaggtgattgattccgaatacgtgccaccggaaagtgacgccgcgggtgccgccggtctccgacgcttccggctacggctggttcgcgaaggacatgtgcgcctggcgctccaaatgatttgtcccttccggaaggacgatccacctgccgcatcgggtacgatcgagctgcaggtgcggccataaagcgggcgctggtccctcatcatagagtgatagccgagtgcccgcacgaactaaggagacagattatggcaatggatgatacgcacgttaggcagctccagtcgatatgccagactcagcatgcgcgctggacgccgggaagcaacaatatgacgagcctcgaacttgaggaagccatacgccatctcgggggagcggttggggacgatgacacgccattcgaggagatggaggcggttggacgtgatctgcacctgcattcgatggcgctacgcagtgctcagtcccgggttcggggcgtgacaccggcggcgacagccccccctgttgagtttgactggcgctcgcacaacgggcgctcttacgtcacctcggtcaagttccagggtgcgtgtggcacgtgcacttgcttcagcacgactgcggccgtggaatcggccatttgcatcgccacccagacttcgccgcagatcattcaaggcgtcgaggttccggctctgtccgaggcgcagatgttctattgcggggcggcgtcacaagaccgcacttgtgcctccggatggtttctcccggcggcgctcgcttacctgcagaacactggggtcgccccgtactcatatttcccctatgaatcgggagaccagccgtgcttgatccaaccgggctgggaatctgtagtgacaaagatcataggctccaccaagctcaccgcccccgatgagattaagtcgtggatcgccaccagagggccagtggcgatcatgatggtggcctatgaggatctcttcacttataaggaagggatttaccacccggtgtcgacgaacaagctcggagtacactcggtgtgcgttgtcggttacagcgacaacaaagaggcgtggctctgcaagaatagctggtcgacacaatggggagaggacggctacttctggatggcctacggcgtatgtggcatgggatcgtccgtacacgggatcaacggtctggccctggttgacggaaagccgctctcgccgcgtcgacccgtcgcctgacacggcgttgcaggggccaatgaacgcagagcgggtgcttaggagggatacatgactcagaatcccggctacagtcttcccgtcgagagggttaacgagctgtcgcgggaacagttccgcaaagactacctcgctcactcccgtccggtcgtcgtcacgggcggcgtccgggagtggcccgcgctgaagcgatgggagcttgagaccctcaccgagcgcctgcaagaccgtacagtggagatcgcctcaaccgccaagggtatcttctcttacgatcttgaatcccccagggctaaatacgaatacatggcattctcggacgcagcagctctcgtggcacagggtcagagggatgcccagtactacatcatgcagctctcgatagaacactacttctccgagctgagagacgatattttgcggctcgacttgctttcgggagaggcgtgttcgccgcacttctggctcggcggggccgatctcgtgacccctttacactgggacaacttgcacaatctttacgggcaggtgcggggacgaaagcgtttcaccctgtttgcgcctgcggaacatgacaatctctatccatacccagctaccgcgctgtacggacacatgtcgtatgcaaaccctgaggcaagtgaacagtggccaaagctgcgcgacgcggagcggtttgaatgcattctggcccccggcgatctgttatttcttccagcgttttggtggcatcacgtccgctcgcttgagctcgcgatctccgtgaacttctggtgggttccgggtctctcggggtgcttcgtcccagccttccttcgcacgctgcggatggcctaccggcgtgaacgtctgacgggtcttggtgcccccgtgtcaacattcccaggagggccgatcggcgcagcccgttcagctctccgcaacgggcaaacgtccttcgcaatgctgttcgcagcctcggccttggagaagacgattcgcgcccgatgctatgcggtcggcattgatgactgggaagatgctacgccgcgcccgatcgaggtgcttgacgctgaacttgcagcttgcggtgcctacccgcccgatctcgaccgcgcgcgccttggctcgtggactcacgccatcaaccgcgtagtcgatggcgactccgagaccgcattgagtgtggcggaggcgacaaccatcgtggacgagatcaggatgttcgtcaccgatatgcactgatcgatcatcctcagccgcaatgcccctgggagggttccgtcgcaaatatgggtgtctcgctgctttggggtgagtgaacctcgtttggtagggtgactacgaggccagagcgtagaactgatcggccacactgagtccctgctcacccctatcttcaagctgcccttttcgcagcatgtgcatcagctcgatgccagtgagagtggactgagcggagtcaaaggattggaaccccagcatcggccgcgtaatccgcttcacacctcgatgatcctgttccacgatgttgttaagatacttgacctggcgaatttcgattgacatgccgtgatccttgttgtagctcttgatggcggcttcgttggccgcactaccatcgatcgtgatcttctccggcacactgccgtggcggccaatcgccttgttgagaaacttcctggccgctttctcatcacgctgctccgtgagcagaaaatcaatggtcttgccgaatttgtcaacagcccggtagaggtatttccactcgcctttcactttaatgtacgtctcatccatccgccaactcgtccacaccgcacgtttgcgccggtggaattcggcttcgagctgcggactgtacttcaccacccagcggttgacggtggaatgatcgacgtggaccccgcgctctgccatgagttcctcaacgtggcgcgtgctcaaagggtacgccagataccatcggactcccatcaaaatgacttcgggtggaaaatgggcacctttgaagctgatcgccat

SEQ ID NO: 2 - DNA, 438 bp, Artificial Sequence
  poyA (precursor peptide)
  CDS (1)..(438): poyA precursor peptide
atggcagacagcgacaatacgcccacatcgaggaaggattttgaaactgcgatcattgcgaaggcctggaaagatccggaatacttgcggcgcctgcggagcaatccgcgtgaagtgctgcaggaagaacttgaggcccttcaccctggggcgcaactccccgacgatctcggaatctcgatccacgaggaagatgaaaatcacgtccatctggtcatgccgcgccatccgcaaaacgtgtcagaccagaccctgacagacgatgacctcgatcaggccgcaggtggcaccggaatcggcgttgtggtggccgtggtcgccggtgccgtcgccaacaccggcgcgggtgtcaatcaggtcgcaggtggcaatatcaatgtggtggggaacatcaacgtcaatgccaacgttagcgtgaatatgaaccagaccacataa

SEQ ID NO: 3 - protein, 145 aa, Artificial Sequence
  Synthetic Construct
MADSDNTPTSRKDFETAIIAKAWKDPEYLRRLRSNPREVLQEELEALHPGAQLPDDLGISIHEEDENHVHLVMPRHPQNVSDQTLTDDDLDQAAGGTGIGVVVAVVAGAVANTGAGVNQVAGGNINVVGNINVNANVSVNMNQTT

SEQ ID NO: 4 - DNA, 1992 bp, Artificial Sequence
  poyB (putative radical SAM methyltransferase)
  CDS (1)..(1992): poyB (putative radical SAM methyltransferase)
atgagtgatgtgcttctagtctcggtaccgtacgcagccttgcagcacccatcccctgcattgggctgtctgcaggccgtgctccggcgccgtgggatcgaagtccacaccatgcatgcgaatctccggttcgctgaacggatcgggatcgggaactatacatggttcggtacctacagccggccacagttgctcggcgagttgacatttgccaaggctgcttttcccgacttcgagcccgatcttcacgcctatgcccaggtcattaacgtaccggaagcggaacatgctatccgcgacgtgcggcaagccgcggtttcgttcatcgacgagatggctcagcagatcatcgagcaagatccgaagattgtgggatgttcctcaacgttccagcagaattgcgcctcgttggcgctcttgcgccggctccgcgagctgtcaccgggcatcgtcaccgtcatgggtggagcaaattgcgaaagcgagatggggcgggctctccatacgaacttcgactgggtagattacgtcgtctcgggtgatggggacgaggtgttccccgacctctgcgagcggattcttcacaacgacattgcgggcatcgcgtctaacgaggcgctgcgccgtttcgtattcacccccgcgtcccgaccgttcgctaattttcaggtggtcgagcgcgccacgactcaggacatggacggattacccctgccggactacgacgattacttccgcgaattggcagagacaaaaatcgaatactttgcgacacctgggcttctcgtcgagaccgcgcgcggttgctggtggggggaaaaacacccgtgcactttttgcggcctcaacggtgggtgcatgagctttcgggcgatgagcccggagaaggcggagtggcacatctgcgaattgtccgcccgctacggcatcgacgggatcgaagtgattgataatatcctcgcgccttcttattttaacaccgttttgccggccctcgcccgaaaggaaaagcgcttgcgcctagcctgcgaggtaaaggccaacctgaagcgggagcaagtgaaagcgctggcggacgcgggggcaatctgggtgcagccgggcatcgaggccctgcacgatgaaacgcttaagctcatcgacaagggcgcgacggtctgccagaacctccagttgctgaaatgggctcgagaatacggcgtccatattacctggaactatctacttggcattccgggcgaacggagggcgttatacaaggaggtcgcggacctcttgcccctcatcatgcatttacaggcacctaatggaccgggctcgcggctcagctttgatcggttcagcgtctatcacacgcatgcagaccattatcagctcacgttgcgtccggcatgggggtattcgaacgtctacccattgccgatcagccagctcatagatctggcctatcacttcgacgatgtcggtcctaacgcggtgctgcgctccgattttccggaaatggaactcttacgagagcgtttgaaggaatggtgcgatctgcagccggagccttccggctatgatgacccggcggatttgcctcccctccccaggctagatgtctttgaacgagacgacggcagactcctcatcgaagacacgcggccgtgcgctccggcgacgcagcgcgagctctgtgggctcagcgcctatctctacaccgcgtgcgaccgaggccgtactgcagcacggcttcggtccgcttgtcacgacgagggcttctctgaggccactgcggccgacgtcgatcgcgagttgacacgcctcattgacgacaagctcatggcttttgtgagcaaccggtttttgagcctcgctgtgcgggcgccataccccgcctgccgacccctgagtgaacaccccgacgggcaggtgtatctacggcccgtccagaagcaccgcgagccgcgcgagcagacgattcaagacgtattcgggcttaagctctga

SEQ ID NO: 5 - protein, 663 aa, Artificial Sequence
  Synthetic Construct
MSDVLLVSVPYAALQHPSPALGCLQAVLRRRGIEVHTMHANLRFAERIGIGNYTWFGTYSRPQLLGELTFAKAAFPDFEPDLHAYAQVINVPEAEHAIRDVRQAAVSFIDEMAQQIIEQDPKIVGCSSTFQQNCASLALLRRLRELSPGIVTVMGGANCESEMGRALHTNFDWVDYVVSGDGDEVFPDLCERILHNDIAGIASNEALRRFVFTPASRPFANFQVVERATTQDMDGLPLPDYDDYFRELAETKIEYFATPGLLVETARGCWWGEKHPCTFCGLNGGCMSFRAMSPEKAEWHICELSARYGIDGIEVIDNILAPSYFNTVLPALARKEKRLRLACEVKANLKREQVKALADAGAIWVQPGIEALHDETLKLIDKGATVCQNLQLLKWAREYGVHITWNYLLGIPGERRALYKEVADLLPLIMHLQAPNGPGSRLSFDRFSVYHTHADHYQLTLRPAWGYSNVYPLPISQLIDLAYHFDDVGPNAVLRSDFPEMELLRERLKEWCDLQPEPSGYDDPADLPPLPRLDVFERDDGRLLIEDTRPCAPATQRELCGLSAYLYTACDRGRTAARLRSACHDEGFSEATAADVDRELTRLIDDKLMAFVSNRFLSLAVRAPYPACRPLSEHPDGQVYLRPVQKHREPREQTIQDVFGLKL

SEQ ID NO: 6 - DNA, 2013 bp, Artificial Sequence
  poyC (putative radical SAM methyltransferase)
  CDS (1)..(2013): poyC (putative radical SAM methyltransferase)
atgccaacaaacgatgtggtcctgctcaacatgccgtattcagccatcgagcacccgtcgatctccctcggatacttctcggccagcctgaagcagcgcggcataagtgtcgacacgatctccgctaatgcgttcttcgcgcgtgatatcgggctcaaggagtactttctcttcagcaactattacaacaacgatttgctcggcgaatggacgttctcgggggaggcattcccggattttcaccccgatcacgacacctatttccgcgatcttcaacttccgattccggaggcgaagatccgtcgcattcgcgagctcgcggcaggtttcatcgaccagatgacagagcgggtcttgtctcagaacccacgcatcgtcggctgcagctcaacctttcagcaaaactgcgcctcgctcgccctgctgcgccgggtgcgggagcaagcgccggaggtcattacagtcatgggcggtgcgaactgtgaggggccaatgggtaaggcgctcaagcagtgtttcgactgggtggatcacgtgttctcaggcgaggcggacgacctttttccggagttctgtgcattgatcctggacgcaactgaccgggagcaagccatgcgccgcatcgctgactgggcgcctgggtcgatctttcaggtctcaacagggctcatgctgcaggcggaagcgagcgagcgctctgtggctaaggatctcagttctctcccaacccccgattacgacgactacttccgcgaccttcaggaagccggggtcgcccggcaggttctgccggggctcatgcttgagacctcgcggggctgctggtggggagagaaagacgtctgtaccttctgtgggttgaatggcgaatacattaacttccgagccaaagaccccgacgtggtgcaccgggagctccgcgatctcacggcccgctacggcatcaatgcgttcgaagtcgtagacaatatcctgtctatgaaatactttaaaacgctcttgccgcagattatcgaatccggagaaaagtatgggttcctttatgaaatcaaggcgaacctgagacgcgatcacgtggagatgctggccgccgctggcgtgctttgggtacaaccgggtatcgagagcctggatgatggggtactccggctgattcacaaaggtgccacggcctgccagaacttgcaactcctcaagtggtgccgcgaatacgggctctttgtcatctggaactacctgtgcgatatccccggtgaacatgacgagtggcatgccgacctggtcgacctcctgccgcaaatcgttcattttcagccgccttcgtcgccgggatcacccttgcgttttgaccgcttcagcgtctatcacaattatccggatgagcatggcctggagctgacccccgcgtggacctactcctatatctatccggtcagcgacgcacacatccagcggatcgcctatttcttcgacaatcaccgcccagatgcggtcaacgatggaaagaaccgcccgctctgggagcaggcgcgtgaggagctcgtggagtggcgcaatctccattaccagtatcaggaagatgacgtgtgggcggaggtggcgtccggctcgccgatgctaagcatgtcatatggcgacggggatctattgatccttgaagacacgcgtccctgcgccgttcaatcgcgaatcgagctccgaggtgccgcggcccgaatttacgagctctgcgacgaaggtcgcaaggcatccaccgtgctgtcgcactgtcggtcgtccggttgtccggatctcgacgcggctgaggtcgaccggattctccaaacattcctagatcacaagattatggcctttgtcagcgaccggtatttgagtcttgcggtgcgtgcgccgtgggcctcgtacccatcgatggagttgtttccaggcgggcgcgttctgctcaagcgcgctgcggctccgaagaaaccccaagaactcacagtcgccgacgtgttcggggtaaaagtctag

SEQ ID NO: 7 - protein, 670 aa, Artificial Sequence
  Synthetic Construct
MPTNDVVLLNMPYSAIEHPSISLGYFSASLKQRGISVDTISANAFFARDIGLKEYFLFSNYYNNDLLGEWTFSGEAFPDFHPDHDTYFRDLQLPIPEAKIRRIRELAAGFIDQMTERVLSQNPRIVGCSSTFQQNCASLALLRRVREQAPEVITVMGGANCEGPMGKALKQCFDWVDHVFSGEADDLFPEFCALILDATDREQAMRRIADWAPGSIFQVSTGLMLQAEASERSVAKDLSSLPTPDYDDYFRDLQEAGVARQVLPGLMLETSRGCWWGEKDVCTFCGLNGEYINFRAKDPDVVHRELRDLTARYGINAFEVVDNILSMKYFKTLLPQIIESGEKYGFLYEIKANLRRDHVEMLAAAGVLWVQPGIESLDDGVLRLIHKGATACQNLQLLKWCREYGLFVIWNYLCDIPGEHDEWHADLVDLLPQIVHFQPPSSPGSPLRFDRFSVYHNYPDEHGLELTPAWTYSYIYPVSDAHIQRIAYFFDNHRPDAVNDGKNRPLWEQAREELVEWRNLHYQYQEDDVWAEVASGSPMLSMSYGDGDLLILEDTRPCAVQSRIELRGAAARIYELCDEGRKASTVLSHCRSSGCPDLDAAEVDRILQTFLDHKIMAFVSDRYLSLAVRAPWASYPSMELFPGGRVLLKRAAAPKKPQELTVADVFGVKV

SEQ ID NO: 8 - DNA, 1608 bp, Artificial Sequence
  poyD (radical SAM-dependent enzyme)
  CDS (1)..(1608): poyD (radical SAM-dependent enzyme)
atgaacctgcagtcgatcgactctcaggcagttcgttcgaaggtggatgcggaatacccgtcggctgcgcatgtcaagcgatttctggaggtttggtgctcagggctctataagaaagaagacttgctcacccgaccgcaggaaatcctcgatgctcaccacgtagcgatagatccgtccctcatctccgttctatttgaatcgaaattcttgcgggggaaatccggcaagtttgacctgctaccgccgcagttcgacgcgtttcgcgactttatgatgacgaagatccagtggcggcaacaaatccgcactggctctgctcctgccgaccccatatttcgtgaatttcgggagcgtcagatccagagatgtgagatcgaactcggcagcgatcagaacaccgccatcgttcacacgcccgtcgtgttcgagctgacgcggggctgctcggtgaagtgttggttttgtgcgctcgacgcaccgcctctcacggggatctttgactattccccggaaaacgcacgatttttccgggacgtgcttcgcgtcgtcaaagacgtgattggtccagcgagcaaatgggccagcagctactggggaaccgacccgctcgacaaccccgatcaagagaaatttagccttgatttccgtgaaatcctcggcatgtatccgcagacgacgacggcgatcccactgcgcgcgcccgagcgcacgcggaagttgcttgaggtgtctcatgccagtggctgcctcgtcaaccgcttctcggttcgtacaccagcgcagctcagaaagattcacgatacgttttcggcagaggagctgctctacaccgagttggtcttgcagaatctggaatcggacagtgtgaaggcgcgtgcgggccgcttgatccatttcgccgatgatctccccaaactcgctgcgaaagatgaggaaaagctgctcaatatgctccaagagcgagaacccgagctagccgcaaaagcaacgtcgattctcattaatctcccaggctccaccgaaccgatcatcagggccacgagtaacgccgatgaagaggatacctccgaggaatacaatgtgtccatcaatgtcccgggcactacgtcctgcctgaccggcttcaagatcaacatggtcgatcgcactgtggagctgttgagcccctgtccagcaaacgagcgatggccgctcggacacattgtgtttgaggaagggacgttcgacacagccgaagacctgaggacgctcatgctcggcatgatctctcgcaatatggctgaacgggtcgttcccgagtcgttggtgcgctttgtcccgcggctcatctatcgcgaagacccggaggggttccgccttggttccgtgttcggcaacggtgtgatctgtcatgacccgactcgatcggcgtacctccatcgtcttggcaacctgctccgcgaggggaaaaagcgagccggcgagatcgcgatgctgtgcttttatgagttcggcgtacctgaaaattacacgatgggaagcatcaacaacatgttccatcagggcatcatcgcggaggagccgatcgccacagaggcgccgatcgccgtggcggcttaccagtaa

SEQ ID NO: 9 - protein, 535 aa, Artificial Sequence
  Synthetic Construct
MNLQSIDSQAVRSKVDAEYPSAAHVKRFLEVWCSGLYKKEDLLTRPQEILDAHHVAIDPSLISVLFESKFLRGKSGKFDLLPPQFDAFRDFMMTKIQWRQQIRTGSAPADPIFREFRERQIQRCEIELGSDQNTAIVHTPVVFELTRGCSVKCWFCALDAPPLTGIFDYSPENARFFRDVLRVVKDVIGPASKWASSYWGTDPLDNPDQEKFSLDFREILGMYPQTTTAIPLRAPERTRKLLEVSHASGCLVNRFSVRTPAQLRKIHDTFSAEELLYTELVLQNLESDSVKARAGRLIHFADDLPKLAAKDEEKLLNMLQEREPELAAKATSILINLPGSTEPIIRATSNADEEDTSEEYNVSINVPGTTSCLTGFKINMVDRTVELLSPCPANERWPLGHIVFEEGTFDTAEDLRTLMLGMISRNMAERVVPESLVRFVPRLIYREDPEGFRLGSVFGNGVICHDPTRSAYLHRLGNLLREGKKRAGEIAMLCFYEFGVPENYTMGSINNMFHQGIIAEEPIATEAPIAVAAYQ

SEQ ID NO: 10 - DNA, 1203 bp, Artificial Sequence
  poyE (SAM-dependent methyltransferase (nucleophilic))
  CDS (1)..(1203): poyE (SAM-dependent methyltransferase (nucleophilic))
atgacgccgaggtcgcttccgaccgagccgctcgcggcccctgccgccgcggctctcgatgccgcacttcgactgctcaaggaatacctggacgatatcggatatcacggcatctacaaatacctggccacggcgaactattatgccgcttcgccctccgtgttcaatgcgtctcgtccccaaagccttgaggcgttcgatcggcaaatccacgagggaccggatgactggatgctcgccagatgcctcacgacgtgcgcgccgtgccgtctggaagctctgccgcccccggcgcgaagggtggccgaggtcctggccgatgtcggcctcctcgtgtggaacgggaacacgcttgagcaaggaggttatcagctcatctctgtgttcgatcgttatattttactcgatgcgcggattcacttcggcggcagtcagctccacgatgtctatattggtcccgacagccacctcttgctgtattacatgccagtggaagcgatcaggccggcagaccacatcctcgacctatgcaccggcaccggggtgatcggacttggcctgtctcgattctccgagcatgtcgtctcaactgacatcgccccgcctgccctgcgtctagcgcatatgaatcgggcactcaacaacgctgaggggcgcgtgtcaattcgcgccgagaatctgcaagagacactcgctagcgatgagtgtttcgatctgatcgcgtgcaatccgccatatgtcgccgcacctcccgagcttcccacgccgctctatgcgcagggtccggatcgggacggcctaggctatctgcgcctgctgatggagcgggctcctgagaagctcaatccgggtgggcaggcgatgtttgtcgtcgatctcattggcgacacgcatcgcccctactacttcgacgacttggagcgcatcgcgaaggagcaagagctgtttatcgaagccttcatcgataaccgtttgaaggcggacgggcagctcccggcttacaagttcctctacgcgcggctgtttcatggcactccccctgaagagatcgaacagcgcatgaggaactttattttcgatgagctgcacgcgtactactattacatgaccacgctccgcgtgcggcgccgcaaaccatccggtttgcgtgtactcgaccggtacaggatcaccagctatgatgagttcttccagcagtcgtga

SEQ ID NO: 11 - protein, 400 aa, Artificial Sequence
  Synthetic Construct
MTPRSLPTEPLAAPAAAALDAALRLLKEYLDDIGYHGIYKYLATANYYAASPSVFNASRPQSLEAFDRQIHEGPDDWMLARCLTTCAPCRLEALPPPARRVAEVLADVGLLVWNGNTLEQGGYQLISVFDRYILLDARIHFGGSQLHDVYIGPDSHLLLYYMPVEAIRPADHILDLCTGTGVIGLGLSRFSEHVVSTDIAPPALRLAHMNRALNNAEGRVSIRAENLQETLASDECFDLIACNPPYVAAPPELPTPLYAQGPDRDGLGYLRLLMERAPEKLNPGGQAMFVVDLIGDTHRPYYFDDLERIAKEQELFIEAFIDNRLKADGQLPAYKFLYARLFHGTPPEEIEQRMRNFIFDELHAYYYYMTTLRVRRRKPSGLRVLDRYRITSYDEFFQQS

SEQ ID NO: 12 - DNA, 1761 bp, Artificial Sequence
  poyF (LanM, N-terminal dehydratase domain)
  CDS (1)..(1761): poyF (LanM, N-terminal dehydratase domain)
atgatgagttcttccagcagtcgtgagggagacgccctcgcccatgctgctgatcccacaaggtcaattgcggacgacatcacttgtgggctcgactcgctaggcggccctcccacggtatgcgatgctgaacatccggtcccatttgaggagctatggctcgggttcgtcgcgcacgcggctcgtgcgcttgaaccgctcatcgcgccgtcgttgcacgcggctgtgttacacgatccgcacggacccctgcgctcgctgctcatcgagctggccgagatgggcgcgccagcggctttcgagttgtttgcactctatcgcatccgggaggcgagtgccgcgtccgtcttcggaagtgtccgcggggtagagagtcgagacacctataacgcattcgtgaagcacctcgccactcagcagctctccccgctgtgggatgcctttccggccctcaagcctctgctggccacccgtaccaatctctttatagccgccatggcagagttgtgccagcggtgggaggccgatcacgtagagatcatgtcggtcttcccggagttacgagggataggtgcaccgcagcgcattcgccccggcttatccgatgcgcatggcggtgggcgaaccgttactcgcctgtcattcgccggtggtgaggcgcttttctacaagccacgcccggtcgacatggagtggggttgtgcacagtttgtcgagtggttcaacggccaagaccacgggatgccgccgctccgggcgctttcggtcttgcccagaaccgactatggctggatggaggcggcacgccccgcactgtgcacacatgttgacgcagtcgcccgattctaccaccgcgcagggatgctgctggccctcgccgaccttttctgcggggtcgatttccatagtgaaaacctgattgcaagcggtgagtatccggtaatcgtggatctggagaccttgtttcatccgctggggcctttcgaatcgcagggcgatgccttggagcgcacagagctgctgccacggcccatctactgtgaagatggtgccccctatgtcatctgcgggttaggtgtggttcctggaaaggcggtcattgagctccggcggcgcggatggatcaacatcaattgcgataacatggcgtcctgtgacgtgaccgtcccttggcctgtcggtggcgccgtgcctcgcaacaaagagggtgcggcgccgtcgatcgcgacatggtcgggcgagatcgtctcggggttctctgctatgcatgtgttcttccgcacacgccggaacgagctgtggagcgccgacgggcctatcgtgcaagcgtttggcggcgggcgttcgcggtttctcctacgcgcgacgcggatctatgtggagctactgcgccgggccgtgcagccgcaagcgctcgcagctcacgcgtcatcgagccacatcttcgatcgtctggcgcgcacgggccggggatgggatgagatccatcgcgtggagcggcaggcgctcgatcgcatggacgtgccttatttcaccatggaaaccaccgcgcagtttgttcgcggcgagggccgtaccatccatgcattcttcgccacgtccgggcttcacatggcacggcgccggtcggagttcatagacgatgtcattctaaatgacagggccgcgtcgattcgcaatgttctcgaacaatcgagttacagcggggaggcgaccctaccggactccagagtccagaccgacgctggcgcaggagcctga

SEQ ID NO: 13 - protein, 586 aa, Artificial Sequence
  Synthetic Construct
MMSSSSSREGDALAHAADPTRSIADDITCGLDSLGGPPTVCDAEHPVPFEELWLGFVAHAARALEPLIAPSLHAAVLHDPHGPLRSLLIELAEMGAPAAFELFALYRIREASAASVFGSVRGVESRDTYNAFVKHLATQQLSPLWDAFPALKPLLATRTNLFIAAMAELCQRWEADHVEIMSVFPELRGIGAPQRIRPGLSDAHGGGRTVTRLSFAGGEALFYKPRPVDMEWGCAQFVEWFNGQDHGMPPLRALSVLPRTDYGWMEAARPALCTHVDAVARFYHRAGMLLALADLFCGVDFHSENLIASGEYPVIVDLETLFHPLGPFESQGDALERTELLPRPIYCEDGAPYVICGLGVVPGKAVIELRRRGWININCDNMASCDVTVPWPVGGAVPRNKEGAAPSIATWSGEIVSGFSAMHVFFRTRRNELWSADGPIVQAFGGGRSRFLLRATRIYVELLRRAVQPQALAAHASSSHIFDRLARTGRGWDEIHRVERQALDRMDVPYFTMETTAQFVRGEGRTIHAFFATSGLHMARRRSEFIDDVILNDRAASIRNVLEQSSYSGEATLPDSRVQTDAGAGA

SEQ ID NO: 14 - DNA, 333 bp, Artificial Sequence
  poyG (chagasin-like peptidase inhibitor)
  CDS (1)..(333): poyG (chagasin-like peptidase inhibitor)
atggataacgcgacacaccatttcgaacagagccattttggcaccttacacgaagtggggctatacgacgaagtgagtattgagcttcttgagtacgctacggccggctttaggtgggagatcacttacgagtcgcccgaagcagtcgaggtgattgattccgaatacgtgccaccggaaagtgacgccgcgggtgccgccggtctccgacgcttccggctacggctggttcgcgaaggacatgtgcgcctggcgctccaaatgatttgtcccttccggaaggacgatccacctgccgcatcgggtacgatcgagctgcaggtgcggccataa

SEQ ID NO: 15 - protein, 110 aa, Artificial Sequence
  Synthetic Construct
MDNATHHFEQSHFGTLHEVGLYDEVSIELLEYATAGFRWEITYESPEAVEVIDSEYVPPESDAAGAAGLRRFRLRLVREGHVRLALQMICPFRKDDPPAASGTIELQVRP

SEQ ID NO: 16 - DNA, 972 bp, Artificial Sequence
  poyH (C1 peptidase)
  CDS (1)..(972): poyH (C1 peptidase)
atggcaatggatgatacgcacgttaggcagctccagtcgatatgccagactcagcatgcgcgctggacgccgggaagcaacaatatgacgagcctcgaacttgaggaagccatacgccatctcgggggagcggttggggacgatgacacgccattcgaggagatggaggcggttggacgtgatctgcacctgcattcgatggcgctacgcagtgctcagtcccgggttcggggcgtgacaccggcggcgacagccccccctgttgagtttgactggcgctcgcacaacgggcgctcttacgtcacctcggtcaagttccagggtgcgtgtggcacgtgcacttgcttcagcacgactgcggccgtggaatcggccatttgcatcgccacccagacttcgccgcagatcattcaaggcgtcgaggttccggctctgtccgaggcgcagatgttctattgcggggcggcgtcacaagaccgcacttgtgcctccggatggtttctcccggcggcgctcgcttacctgcagaacactggggtcgccccgtactcatatttcccctatgaatcgggagaccagccgtgcttgatccaaccgggctgggaatctgtagtgacaaagatcataggctccaccaagctcaccgcccccgatgagattaagtcgtggatcgccaccagagggccagtggcgatcatgatggtggcctatgaggatctcttcacttataaggaagggatttaccacccggtgtcgacgaacaagctcggagtacactcggtgtgcgttgtcggttacagcgacaacaaagaggcgtggctctgcaagaatagctggtcgacacaatggggagaggacggctacttctggatggcctacggcgtatgtggcatgggatcgtccgtacacgggatcaacggtctggccctggttgacggaaagccgctctcgccgcgtcgacccgtcgcctga

SEQ ID NO: 17 - protein, 323 aa, Artificial Sequence
  Synthetic Construct
MAMDDTHVRQLQSICQTQHARWTPGSNNMTSLELEEAIRHLGGAVGDDDTPFEEMEAVGRDLHLHSMALRSAQSRVRGVTPAATAPPVEFDWRSHNGRSYVTSVKFQGACGTCTCFSTTAAVESAICIATQTSPQIIQGVEVPALSEAQMFYCGAASQDRTCASGWFLPAALAYLQNTGVAPYSYFPYESGDQPCLIQPGWESVVTKIIGSTKLTAPDEIKSWIATRGPVAIMMVAYEDLFTYKEGIYHPVSTNKLGVHSVCVVGYSDNKEAWLCKNSWSTQWGEDGYFWMAYGVCGMGSSVHGINGLALVDGKPLSPRRPVA

SEQ ID NO: 18 - DNA, 1173 bp, Artificial Sequence
  poyI (Fe(II)/alpha-ketoglutarate-dependent oxygenase)
  CDS (1)..(1173): poyI (Fe(II)/alpha-ketoglutarate-dependent oxygenase)
atgactcagaatcccggctacagtcttcccgtcgagagggttaacgagctgtcgcgggaacagttccgcaaagactacctcgctcactcccgtccggtcgtcgtcacgggcggcgtccgggagtggcccgcgctgaagcgatgggagcttgagaccctcaccgagcgcctgcaagaccgtacagtggagatcgcctcaaccgccaagggtatcttctcttacgatcttgaatcccccagggctaaatacgaatacatggcattctcggacgcagcagctctcgtggcacagggtcagagggatgcccagtactacatcatgcagctctcgatagaacactacttctccgagctgagagacgatattttgcggctcgacttgctttcgggagaggcgtgttcgccgcacttctggctcggcggggccgatctcgtgacccctttacactgggacaacttgcacaatctttacgggcaggtgcggggacgaaagcgtttcaccctgtttgcgcctgcggaacatgacaatctctatccatacccagctaccgcgctgtacggacacatgtcgtatgcaaaccctgaggcaagtgaacagtggccaaagctgcgcgacgcggagcggtttgaatgcattctggcccccggcgatctgttatttcttccagcgttttggtggcatcacgtccgctcgcttgagctcgcgatctccgtgaacttctggtgggttccgggtctctcggggtgcttcgtcccagccttccttcgcacgctgcggatggcctaccggcgtgaacgtctgacgggtcttggtgcccccgtgtcaacattcccaggagggccgatcggcgcagcccgttcagctctccgcaacgggcaaacgtccttcgcaatgctgttcgcagcctcggccttggagaagacgattcgcgcccgatgctatgcggtcggcattgatgactgggaagatgctacgccgcgcccgatcgaggtgcttgacgctgaacttgcagcttgcggtgcctacccgcccgatctcgaccgcgcgcgccttggctcgtggactcacgccatcaaccgcgtagtcgatggcgactccgagaccgcattgagtgtggcggaggcgacaaccatcgtggacgagatcaggatgttcgtcaccgatatgcactga

SEQ ID NO: 19 - protein, 390 aa, Artificial Sequence
  Synthetic Construct
MTQNPGYSLPVERVNELSREQFRKDYLAHSRPVVVTGGVREWPALKRWELETLTERLQDRTVEIASTAKGIFSYDLESPRAKYEYMAFSDAAALVAQGQRDAQYYIMQLSIEHYFSELRDDILRLDLLSGEACSPHFWLGGADLVTPLHWDNLHNLYGQVRGRKRFTLFAPAEHDNLYPYPATALYGHMSYANPEASEQWPKLRDAERFECILAPGDLLFLPAFWWHHVRSLELAISVNFWWVPGLSGCFVPAFLRTLRMAYRRERLTGLGAPVSTFPGGPIGAARSALRNGQTSFAMLFAASALEKTIRARCYAVGIDDWEDATPRPIEVLDAELAACGAYPPDLDRARLGSWTHAINRVVDGDSETALSVAEATTIVDEIRMFVTDMH

SEQ ID NO: 20 - DNA, 1737 bp, Artificial Sequence
  poyJ (putative transporter/hydrolase)
  CDS (1)..(1737): poyJ (putative transporter/hydrolase)
atgagccaatccaatagaatgggtacgacgccagcggacgccactgaagccggtaccgactccagtgccgctcccgccggggacgcttctgcggatcaagaggtggctcctggctgggttgccgttttgtttcccaacggcgagccgctcgtctatgcattgtcgattgccgatgagcgctatgcgacgcagtggaccgtgtaccgggggccacgcgagggtgatctccacgagattgaattcttccttgagcttgatccggtggcgatgggtctagggggagaacttgtgaggcaacgcagcgtcttgctctgcaacggcgcactcgatcccgtgcactacctcagcgaggcaggtggtactcgctgggaggtccgctttgaatcggccgaggtgacggccacgcttcccgatggatcggagcaagtcgtgccgcgcggggaggcgcagttcatcgccgccgacaatgtgccggggcacaaggcactgatctttgccgccttggcatgccgcaagctcctcgaccaagaggtgacagttggactctttctcgccaatcagctggcggccgttccctaccagatgagtcccgcactcgaccttgccgccgagtccggcacgtggcaccgctcctcgcatcttgaagagattcacctcgatgaacacgggttgatgatcgaaggcatggtgccgacgcaaggggtccgcggatggctggagcgccctggtccgccagtgccgcaatggcttgacgaggatcagccggtcaccgtcccgctccgctaccactatcgcgacaatacccgcttccgtcttgaggacgtcactatccccggcccggtgacgccaatcggtgcgacgctctccattccggaaggtccgggccccttcccagccgtgctcttcctgagtggttcgggcactcatgatcgtcacgggatcgcgggtgaaatagatatgggaacgcacgaaattatggactttttggcggagcaaggccttctggggttgcgtttcgattcccgcggggcgggcacgacgaaaatgggcgaggacactctggttcgcggcctcgattcggaaattgctgatgcccgggcatgcctagaatttctgcgcgctcgcccggaagcggctggaggtgccgttttcctccttggccatagccagggcggcactgtggctctggtgctcgccgagcaagagggtattgccctgcgcggagtggtcttgatggccaccatgggccggagcatcgaggacgtcatcaccgaccagattgtctacatggggaaagacatcggcttgacggacgagcaaatcgagcagcagattcaggaggttcaggaggcggtagagttggtgaaggcagatcatccgtggaatccggacaatatcccccactacctcctcgcgatgttccgcagcccgacttggctgaaacagttattgttataccggacagtggaactcattacccggctccagtgcccactactcgtgtgtcagggtagcaaagacttccaagtctccgccgagcgcgatgccgagctgctcgttaccgcagctcaaagtgcgggcggtgattgtacgtatgcgctcttccccaatctcgatcacctcttcaaggcgacagctggggaatcgacaatggcccagtacttcgatcaaacccgccacgtcgatccggagttcctccagcgcgtcgcagcctggctgagcgaacacgcgtcctga

SEQ ID NO: 21 - protein, 578 aa, Artificial Sequence
  Synthetic Construct
MSQSNRMGTTPADATEAGTDSSAAPAGDASADQEVAPGWVAVLFPNGEPLVYALSIADERYATQWTVYRGPREGDLHEIEFFLELDPVAMGLGGELVRQRSVLLCNGALDPVHYLSEAGGTRWEVRFESAEVTATLPDGSEQVVPRGEAQFIAADNVPGHKALIFAALACRKLLDQEVTVGLFLANQLAAVPYQMSPALDLAAESGTWHRSSHLEEIHLDEHGLMIEGMVPTQGVRGWLERPGPPVPQWLDEDQPVTVPLRYHYRDNTRFRLEDVTIPGPVTPIGATLSIPEGPGPFPAVLFLSGSGTHDRHGIAGEIDMGTHEIMDFLAEQGLLGLRFDSRGAGTTKMGEDTLVRGLDSEIADARACLEFLRARPEAAGGAVFLLGHSQGGTVALVLAEQEGIALRGVVLMATMGRSIEDVITDQIVYMGKDIGLTDEQIEQQIQEVQEAVELVKADHPWNPDNIPHYLLAMFRSPTWLKQLLLYRTVELITRLQCPLLVCQGSKDFQVSAERDAELLVTAAQSAGGDCTYALFPNLDHLFKATAGESTMAQYFDQTRHVDPEFLQRVAAWLSEHAS

SEQ ID NO: 22 - DNA, 351 bp, Artificial Sequence
  poyK (unknown)
  CDS (1)..(351): poyK (unknown)
atgcttcagacccaatgtttgaaagtaccaatcatcccaggcaaagaggctgaggtcaaaaattggctcgccgcgctttcgactcgacaccaggaggtgctggaagcgatcacatcggaagacatcgctgatgaggccatgttttacgcgaaagaaccatccggcgagttcctctacttatactctcgtgcaccggacttggcagtagctggcgccgcgtttcagaaatcccaattgcccatcgatcaagagttcaagcgaatctccgcggaatgtctggactaccgttcagccatccgacttgaactccttctttctgcggatagccgcaacaagtatgtgtatccctaa

SEQ ID NO: 23 - protein, 116 aa, Artificial Sequence
  Synthetic Construct
MLQTQCLKVPIIPGKEAEVKNWLAALSTRHQEVLEAITSEDIADEAMFYAKEPSGEFLYLYSRAPDLAVAGAAFQKSQLPIDQEFKRISAECLDYRSAIRLELLLSADSRNKYVYP

SEQ ID NO: 24 - DNA, 684 bp, Artificial Sequence
  ORF-1 (integrase; outside of poy gene cluster, just downstream of poyI)
  CDS (1)..(684): ORF-1 (integrase; outside of poy gene cluster, just downstream of poyI)
atggcgatcagcttcaaaggtgcccattttccacccgaagtcattttgatgggagtccgatggtatctggcgtaccctttgagcacgcgccacgttgaggaactcatggcagagcgcggggtccacgtcgatcattccaccgtcaaccgctgggtggtgaagtacagtccgcagctcgaagccgaattccaccggcgcaaacgtgcggtgtggacgagttggcggatggatgagacgtacattaaagtgaaaggcgagtggaaatacctctaccgggctgttgacaaattcggcaagaccattgattttctgctcacggagcagcgtgatgagaaagcggccaggaagtttctcaacaaggcgattggccgccacggcagtgtgccggagaagatcacgatcgatggtagtgcggccaacgaagccgccatcaagagctacaacaaggatcacggcatgtcaatcgaaattcgccaggtcaagtatcttaacaacatcgtggaacaggatcatcgaggtgtgaagcggattacgcggccgatgctggggttccaatcctttgactccgctcagtccactctcactggcatcgagctgatgcacatgctgcgaaaagggcagcttgaagataggggtgagcagggactcagtgtggccgatcagttctacgctctggcctcgtag

SEQ ID NO: 25 - protein, 227 aa, Artificial Sequence
  Synthetic Construct
MAISFKGAHFPPEVILMGVRWYLAYPLSTRHVEELMAERGVHVDHSTVNRWVVKYSPQLEAEFHRRKRAVWTSWRMDETYIKVKGEWKYLYRAVDKFGKTIDFLLTEQRDEKAARKFLNKAIGRHGSVPEKITIDGSAANEAAIKSYNKDHGMSIEIRQVKYLNNIVEQDHRGVKRITRPMLGFQSFDSAQSTLTGIELMHMLRKGQLEDRGEQGLSVADQFYALAS

SEQ ID NO: 26 - DNA, 213 bp, Artificial Sequence
  ORF-2 (toxin-antitoxin system: toxin component; outside of poy gene cluster, just upstream of poyK)
  CDS (1)..(213): ORF-2 (toxin-antitoxin system: toxin component; outside of poy gene cluster, just upstream of poyK)
atgactggcaacgaattcattcgacgactaagaaggcttggacgtcaacgcggcgtcagggtcgaatttgtccccgagcgtggtaagggaagccacgggaccttgtactatggcgaacggctcaccattgtgcgcaatcccagagatgaattgaaaacggggacgctctatgccatgctcgcccaacttggcctgagccgtgcggatttatag

SEQ ID NO: 27 - protein, 70 aa, Artificial Sequence
  Synthetic Construct
MTGNEFIRRLRRLGRQRGVRVEFVPERGKGSHGTLYYGERLTIVRNPRDELKTGTLYAMLAQLGLSRADL

SEQ ID NO: 28 - DNA, 567 bp, Artificial Sequence
  M. marina ATCC 23134 precursor peptide
  CDS (1)..(567): M. marina ATCC 23134 precursor peptide
atgacccaacaagagatattagacagattcggtagtttggaaaagttgattacggacaccaacttccgaaacgccctcaaaaaagatcctcgtaaagcccttgcacaagaactttctggtgtgaccattcctgataatgtaagcctcatcgtgcatgagaacaccaccaatgaaatgcacattattttattgcctgatgctgaagtttcgggagaagacatgcccgatgatgacccaatggaagtggtgttagacaaggcaatggcagataaaagtttcaaagatttgctgatgatagaccccaaaggtgtgttggcaaaagagcttccagatttttatgtacctgacgagtttaaggtatattttcacgaaaatacagcgaccgaatggcacttgttgattccatcgttggaaactgaagatgaagatggtgagctaagtgaagatgagcttgaggcagttgccggaggagctggacgaaggcgccgccgccgccgccgtggcccccatattggtcgtcgtcgtggaggcaaaggtcctcgttgccgtaagagaagattccgttaa

SEQ ID NO: 29 - protein, 188 aa, Artificial Sequence
  Synthetic Construct
MTQQEILDRFGSLEKLITDTNFRNALKKDPRKALAQELSGVTIPDNVSLIVHENTTNEMHIILLPDAEVSGEDMPDDDPMEVVLDKAMADKSFKDLLMIDPKGVLAKELPDFYVPDEFKVYFHENTATEWHLLIPSLETEDEDGELSEDELEAVAGGAGRRRRRRRRGPHIGRRRGGKGPRCRKRRFR

SEQ ID NO: 30 - DNA, 264 bp, Artificial Sequence
  D. baarsi DSM 2075 precursor peptide
  CDS (1)..(264): D. baarsi DSM 2075 precursor peptide
atgtcctcggataatatggcgcattccagcgcgtgggccaaggtcgtggccaaggcctgggccgacgagtcttacaaaaataagctgctcagcgatccggcggcggtgctgcgcgccgagggcttggccatccccgagggcgtgcgcctgacggtgctggaaaacagcgccacccagatccatctggtgctgccggtcgcgcccagtgacgcggccgacctggaagacgccgccctgggcgagcgcctggccgccgtaatctag

SEQ ID NO: 31 - protein, 87 aa, Artificial Sequence
  Synthetic Construct
MSSDNMAHSSAWAKVVAKAWADESYKNKLLSDPAAVLRAEGLAIPEGVRLTVLENSATQIHLVLPVAPSDAADLEDAALGERLAAVI

SEQ ID NO: 32 - DNA, 345 bp, Artificial Sequence
  C. luteolum DSM 273 precursor peptide
  CDS (1)..(345): C. luteolum DSM 273 precursor peptide
atggcgtgcaatagtgatagatcattgttgttatctcaaccaaagaccaaggatgtccccatggaagcaaacgaacagcagcaggcactgggcaagatcatagccaacgcctgggcggatgaaggtttcaagcagcagttcatcgaaaaccctgcagagatcctgagagcagagggtatcagtgtgccggatggaatgatggtcaacgtgatggagaataccccgacctgcatgcatatcgtcctcccgcaatccccggacattgatctggatggggctgctctggacgcacttgccggcggagagtatgtattatgtagtggtggttggtgtcagcaggagtga

SEQ ID NO: 33 - protein, 114 aa, Artificial Sequence
  Synthetic Construct
MACNSDRSLLLSQPKTKDVPMEANEQQQALGKIIANAWADEGFKQQFIENPAEILRAEGISVPDGMMVNVMENTPTCMHIVLPQSPDIDLDGAALDALAGGEYVLCSGGWCQQE

SEQ ID NO: 34 - DNA, 357 bp, Artificial Sequence
  N. punctiforme PCC 73102 precursor peptide 1
  CDS (1)..(357): N. punctiforme PCC 73102 precursor peptide 1
atgagcgaacaagaacaagcgcaaactcgcaaaaacatcgaagcccggattgttgccaaagcctggaaagatgaagggtataaacaagaattgcttaccaatcccaaagctataatcgagcgggaatttggagtggaattccctgctgaagttagcgtacaagtcctagaagagaattccacttctttgtattttgtactgccaattagtccagtagcgatcgctcaagaattatctgaagagcaactagaagcgatcgctggtggttatatgacaactctcgcatctgcaaacgcatccgcaaaaataaatcccattttgcccatacgacactcacttgtgaaaacacttagataa

SEQ ID NO: 35 - protein, 118 aa, Artificial Sequence
  Synthetic Construct
MSEQEQAQTRKNIEARIVAKAWKDEGYKQELLTNPKAIIEREFGVEFPAEVSVQVLEENSTSLYFVLPISPVAIAQELSEEQLEAIAGGYMTTLASANASAKINPILPIRHSLVKTLR

SEQ ID NO: 36 - DNA, 405 bp, Artificial Sequence
  N. punctiforme PCC 73102 precursor peptide 2
  CDS (1)..(405): N. punctiforme PCC 73102 precursor peptide 2
atgactcaacaagaacaagcgcaaacacgccaagatatcgaagcccgcatcatcgccaaagcttggaaagatgaagcatacaaacaagagttattaaccaatcccaaagctgtaattgagcgggaatttggagtggaatttcctgctgatgttaacgtgcaagtccttgaagagaatcctacctctttgcattttgtactaccaattagtccggtagcaatcgcccaagaattatctgaagaggaattactagcgctcgctgctggtgtaaattattcggccgtgaccgtagcgattgtcaaaaataccgttaaacaaaacacaaatataatcacgagagctgctgtctcagtaacagccttagtcactggagcctctataggcgcttcaagcgtacatctctaa

SEQ ID NO: 37 - protein, 134 aa, Artificial Sequence
  Synthetic Construct
MTQQEQAQTRQDIEARIIAKAWKDEAYKQELLTNPKAVIEREFGVEFPADVNVQVLEENPTSLHFVLPISPVAIAQELSEEELLALAAGVNYSAVTVAIVKNTVKQNTNIITRAAVSVTALVTGASIGASSVHL

SEQ ID NO: 38 - DNA, 336 bp, Artificial Sequence
  Nostoc sp. PCC 7120 precursor peptide
  CDS (1)..(336): Nostoc sp. PCC 7120 precursor peptide
atgagtgagcaaactaaaactcgtaaagatgttgaagcacaaatcattgtgcaagcatggaaagatgaagcttacagacaggaattactgaacaatcctaaaaaaatagttgaacaagaatttggtgttcaattaccagagggaataacagttcacgtcatggaagaaaatgcttctaacctctattttgtaattcctgcacgccctaacttagaagatgtagaattatcagatgagcagctagaagccgttgctggtggagcgttatggacacttacgctccttttaattcctatcgctcatggcgctcttgaagaacataattctagaaaataa

SEQ ID NO: 39 - protein, 111 aa, Artificial Sequence
  Synthetic Construct
MSEQTKTRKDVEAQIIVQAWKDEAYRQELLNNPKKIVEQEFGVQLPEGITVHVMEENASNLYFVIPARPNLEDVELSDEQLEAVAGGALWTLTLLLIPIAHGALEEHNSRK

SEQ ID NO: 40 - DNA, 297 bp, Artificial Sequence
  Oscillatoria sp. PCC 6506 precursor peptide 1
  CDS (1)..(297): Oscillatoria sp. PCC 6506 precursor peptide 1
atgtctactcgcaaagaagccgaagaacaactcgccatcaaagctcttaaagatcccagcttccgcgaaaaactcaaagccaatcctaaagcagtgatttcttcagagtttaacactcaagtgccagacgatctgacaattgaagtagtagaagaaacagctactaagatgtacttagttctacctgctcctgaagctgttgaagaagaattatctgaagaacaattagaagctgtcgctggtggcggttgctggattgctggtagccgtggctgcggttttgtaactcgcacttaa

SEQ ID NO: 41 - protein, 98 aa, Artificial Sequence
  Synthetic Construct
MSTRKEAEEQLAIKALKDPSFREKLKANPKAVISSEFNTQVPDDLTIEVVEETATKMYLVLPAPEAVEEELSEEQLEAVAGGGCWIAGSRGCGFVTRT

SEQ ID NO: 42 - DNA, 339 bp, Artificial Sequence
  Oscillatoria sp. PCC 6506 precursor peptide 2
  CDS (1)..(339): Oscillatoria sp. PCC 6506 precursor peptide 2
atgacttcatcaacatctcaaccggaaccaatgactcgtgaagaactacaagccaaactgattgccaaagcttggcaggacgagtcatttaagcaagaactactcagtaaccccacagcagtcattgctaaggaaatgggtgtggataatatccctggaatcaccatccagatagtagaagaaacccctactacctattacctagtgttgccatctaaaccaacggatgacaccgaagaactttctgatgcggaattggaagctatcgcaggtggtcgtcgcaggggtgggagtagccgggtgattaccaacaccccaggcgtgcctggttgcaattag

SEQ ID NO: 43 - protein, 112 aa, Artificial Sequence
  Synthetic Construct
MTSSTSQPEPMTREELQAKLIAKAWQDESFKQELLSNPTAVIAKEMGVDNIPGITIQIVEETPTTYYLVLPSKPTDDTEELSDAELEAIAGGRRRGGSSRVITNTPGVPGCN

SEQ ID NO: 44 - DNA, 399 bp, Artificial Sequence
  Azospirillum sp. B510 precursor peptide
  CDS (1)..(399): Azospirillum sp. B510 precursor peptide
atgacagaccaaacgcagtccgccccgatgacccgccgcgaccttgaggcgaagatcgtcgcccgcgcctggtcggacgacgacttcaaggcgaagttcctggccgaccccaaggcgatgttcgaggagcatctgggcaccaaactacccgcctcgctggtgatgacggcgcacgaggaaaccgccgacacgatccacttcgtcatcccggccaagccgcggatcgacctggacgagctgtcggacgaggatctggagaaggtggccggcggcgtggacatcgtgacgacgatcaccgtcaccgcgatcatctcggccggtgtgggcggtgccgccttctcggcggtggcgaccgttctcgccgccggtggaatcaggggggtgtgtgcaaaatggtag

SEQ ID NO: 45 - protein, 132 aa, Artificial Sequence
  Synthetic Construct
MTDQTQSAPMTRRDLEAKIVARAWSDDDFKAKFLADPKAMFEEHLGTKLPASLVMTAHEETADTIHFVIPAKPRIDLDELSDEDLEKVAGGVDIVTTITVTAIISAGVGGAAFSAVATVLAAGGIRGVCAKW

SEQ ID NO: 46 - DNA, 339 bp, Artificial Sequence
  P. thermopropionicum SI precursor peptide
  CDS (1)..(339): P. thermopropionicum SI precursor peptide
atgatcgaaagcgaaaagaaacccgtgacccgcaaagaattgaaggagcaaatcatcaggaaagcgcaggaagaccgggaatttaagaaagcattggtcgggaatcccaaaggagccgttgaacaattgggcgtccaacttcccgaagacgttgaggtcaaagtcgttgaggaatccgcagaggtggtttatctggtgctgccggtcaatcccggcgagttgaccggtgagcagttggataatgtagcgggcgggaccggctgttccgatgtatattcctttcctatctgcgttcccacctaccatgacaacacggtaccggcaccaaaggcagggtag

SEQ ID NO: 47 - protein, 112 aa, Artificial Sequence
  Synthetic Construct
MIESEKKPVTRKELKEQIIRKAQEDREFKKALVGNPKGAVEQLGVQLPEDVEVKVVEESAEVVYLVLPVNPGELTGEQLDNVAGGTGCSDVYSFPICVPTYHDNTVPAPKAG

SEQ ID NO: 48 - protein, 49 aa, Artificial Sequence
  Cleaved form of the PoyA precursor peptide
TGIGVVVAVVAGAVANTGAGVNQVAGGNINVVGNINVNANVSVNMNQTT

SEQ ID NO: 49 - protein, 31 aa, Artificial Sequence
  Cleaved form of the M. marina ATCC 23134 precursor peptide
AGRRRRRRRRGPHIGRRRGGKGPRCRKRRFR

SEQ ID NO: 50 - protein, 7 aa, Artificial Sequence
  Cleaved form of the D. baarsi DSM 2075 precursor peptide
ERLAAVI

SEQ ID NO: 51 - protein, 13 aa, Artificial Sequence
  Cleaved form of the C. luteolum DSM 273 precursor peptide
EYVLCSGGWCQQE

SEQ ID NO: 52 - protein, 29 aa, Artificial Sequence
  Cleaved form of the N. punctiforme PCC 73102 precursor peptide 1
YMTTLASANASAKINPILPIRHSLVKTLR

SEQ ID NO: 53 - protein, 45 aa, Artificial Sequence
  Cleaved form of the N. punctiforme PCC 73102 precursor peptide 2
VNYSAVTVAIVKNTVKQNTNIITRAAVSVTALVTGASIGASSVHL

SEQ ID NO: 54 - protein, 24 aa, Artificial Sequence
  Cleaved form of the Nostoc sp. PCC 7120 precursor peptide
ALWTLTLLLIPIAHGALEEHNSRK

SEQ ID NO: 55 - protein, 14 aa, Artificial Sequence
  Cleaved form of the Oscillatoria sp. PCC 6506 precursor peptide 1
WIAGSRGCGFVTRT

SEQ ID NO: 56 - protein, 15 aa, Artificial Sequence
  Cleaved form of the Oscillatoria sp. PCC 6506 precursor peptide 2
SSRVITNTPGVPGCN

SEQ ID NO: 57 - protein, 41 aa, Artificial Sequence
  Cleaved form of the Azospirillum sp. B510 precursor peptide
VDIVTTITVTAIISAGVGGAAFSAVATVLAAGGIRGVCAKW

SEQ ID NO: 58 - protein, 27 aa, Artificial Sequence
  Cleaved form of the P. thermopropionicum SI precursor peptide
TGCSDVYSFPICVPTYHDNTVPAPKAG

SEQ ID NO: 59 - protein, 15 aa, Artificial Sequence
  UZ-HT15 his-tag alternative
MKHQHQHQHQHQHQQ

SEQ ID NO: 60 - protein, 15 aa, Artificial Sequence
  S-tag
KETAAAKFERQHMDS

SEQ ID NO: 61 - DNA, 20 bp, Artificial Sequence
  Polytheo-For1: degenerated oligonucleotide sequence for peptide sequence GIGVVVA
  misc_feature (3)..(3): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (18)..(18): n is a, c, g, or t
ggnathggngtngtngtngc

SEQ ID NO: 62 - DNA, 20 bp, Artificial Sequence
  Polytheo-For2: degenerated oligonucleotide sequence for peptide sequence GAGVNQT
  misc_feature (3)..(3): n is a, c, g, or t
  misc_feature (6)..(6): n is a, c, g, or t
  misc_feature (9)..(9): n is a, c, g, or t
  misc_feature (12)..(12): n is a, c, g, or t
ggngcnggngtnaaycarac

SEQ ID NO: 63 - DNA, 20 bp, Artificial Sequence
  Polytheo-Rev: degenerated oligonucleotide sequence for peptide sequence for peptide sequence VNMNQTT
  misc_feature (3)..(3): n is a, c, g, or t
  misc_feature (18)..(18): n is a, c, g, or t
gtngtytgrttcatrttnac

SEQ ID NO: 64 - DNA, 19 bp, Artificial Sequence
  PolytheoFor1 - nested primer (alias PolytheoXFor1)
ccggtgccgtcgccaacac

SEQ ID NO: 65 - DNA, 21 bp, Artificial Sequence
  PolytheoFor2: - nested primer (alias PolytheoXFor2)
gcgggtgtcaatcaggtcgca

SEQ ID NO: 66 - DNA, 26 bp, Artificial Sequence
  PolytheoRev1: - nested primer (alias PolytheoXRev1)
gacgttgatgttccccaccacattga

SEQ ID NO: 67 - DNA, 24 bp, Artificial Sequence
  PolytheoRev2: - nested primer (alias PolytheoXRev2)
cgttggcattgacgttgatgttcc

SEQ ID NO: 68 - protein, 7 aa, Artificial Sequence
  target peptide sequence of degenerated oligonucleotide Polytheo-For1
GIGVVVA

SEQ ID NO: 69 - protein, 7 aa, Artificial Sequence
  target peptide sequence of degenerated oligonucleotide Polytheo-For2
GAGVNQT

SEQ ID NO: 70 - protein, 7 aa, Artificial Sequence
  target peptide sequence of degenerated oligonucleotide Polytheo-Rev
VNMNQTT

SEQ ID NO: 71 - DNA, 20 bp, Artificial Sequence
  Oligonucleotide/Primer pWEBfor
cgccagggttttcccagtca

SEQ ID NO: 72 - DNA, 21 bp, Artificial Sequence
  Oligonucleotide/Primer Up-PolyTheo-Rev1
aggtggtactcgctgggaggt

SEQ ID NO: 73 - DNA, 21 bp, Artificial Sequence
  Oligonucleotide/Primer Up-PolyTheo-Rev2
agaacttgtgaggcaacgcag

SEQ ID NO: 74 - DNA, 29 bp, Artificial Sequence
  Oligonucleotide/Primer PT-opt-F
caagacatatggcagacagcgacaacacc

SEQ ID NO: 75 - DNA, 33 bp, Artificial Sequence
  Oligonucleotide/Primer PT-opt-R
agcaagcttttaggtggtctgattcatgttcac

SEQ ID NO: 76 - DNA, 30 bp, Artificial Sequence
  Oligonucleotide/Primer PT-opt-F3
catgaattctatggcagacagcgacaacac

SEQ ID NO: 77 - DNA, 29 bp, Artificial Sequence
  Oligonucleotide/Primer SAM1-R
gatgtcgactcagagcttcagcccgaata

SEQ ID NO: 78 - DNA, 31 bp, Artificial Sequence
  Oligonucleotide/Primer SAM2-F
gcaagaattaatgccaacaaacgatgtggtc

SEQ ID NO: 79 - DNA, 30 bp, Artificial Sequence
  Oligonucleotide/Primer SAM2-R
cctcgagctagacttttaccccgaacacgt

SEQ ID NO: 80 - DNA, 32 bp, Artificial Sequence
  Oligonucleotide/Primer SAM3-F
caagacatatgaacctgcagtcgattgactct

SEQ ID NO: 81 - DNA, 26 bp, Artificial Sequence
  Oligonucleotide/Primer SAM3-R
gctcgagttactggtaagccgccacg

SEQ ID NO: 82 - DNA, 27 bp, Artificial Sequence
  Oligonucleotide/Primer NmethT-F
caagacatatgacgccgaggtcgcttc

SEQ ID NO: 83 - DNA, 28 bp, Artificial Sequence
  Oligonucleotide/Primer NmethT-R
gctcgagtcacgactgctggaagaactc

SEQ ID NO: 84 - DNA, 33 bp, Artificial Sequence
  Oligonucleotide/Primer Lanth-F
caagacatatgatgagttcttccagcagtcgtg

SEQ ID NO: 85 - DNA, 23 bp, Artificial Sequence
  Oligonucleotide/Primer Lanth-R
tctcgagtcaggctcctgcgcca

SEQ ID NO: 86 - DNA, 26 bp, Artificial Sequence
  Oligonucleotide/Primer NMethT-opt-F
caagacatatgacgccgcgtagcctg

SEQ ID NO: 87 - DNA, 32 bp, Artificial Sequence
  Oligonucleotide/Primer NMethT-opt-R
ggcaagctttaagattgctggaaaaactcatc

SEQ ID NO: 88 - DNA, 28 bp, Artificial Sequence
  Oligonucleotide/Primer PT-opt-half-R
agcaagcttttaggccacctgattgacg

SEQ ID NO: 89 - DNA, 44 bp, Artificial Sequence
  Oligonucleotide/Primer PT_OPT_R_ADD_1_9
attaagcttttagacaccaatacccgtgccacccgctgcttgat

SEQ ID NO: 90 - DNA, 438 bp, Artificial Sequence
  for E.coli expression codon optimized poyA
  CDS (1)..(435): for E.coli expression codon optimized poyA
atggcagacagcgacaacaccccgacgtcccgcaaagatttcgagactgccattattgcgaaagcatggaaggacccggaatacttgcgccgtctgcgtagcaatccgcgtgaggtgctgcaagaagagttggaggctctgcacccaggtgcacagctgccggacgatctgggcatctctatccacgaagaggacgaaaaccacgttcatctggtcatgccgcgtcatccgcagaatgttagcgatcaaaccctgacggatgatgacctggatcaagcagcgggtggcacgggtattggtgtcgttgtggcggtggttgcgggtgccgttgctaacaccggtgcgggcgtcaatcaggtggccggtggcaacatcaatgtcgtgggcaatatcaacgttaacgcgaatgtcagcgtgaacatgaatcagaccacctaa

SEQ ID NO: 91 - protein, 145 aa, Artificial Sequence; identical to SEQ ID NO: 3
  Synthetic Construct

SEQ ID NO: 92 - DNA, 1203 bp, Artificial Sequence
  for E.coli expression codon optimized poyE
  CDS (1)..(1200): for E.coli expression codon optimized poyE
atgacgccgcgtagcctgccgaccgaaccactggccgcaccagcagccgcagcattggacgcagctctgcgcctgctgaaagaatacctggacgatatcggttaccacggtatttacaaatatctggcgaccgctaactattatgcagctagcccgagcgtctttaatgcgagccgtccgcagtccctggaggcgttcgatcgtcaaatccacgagggtccggacgattggatgctggcgcgctgtctgaccacctgcgcaccgtgtcgtctggaggccttgccgcctccggcacgtcgtgttgccgaggttctggcggacgttggtctgttggtctggaatggtaataccctggaacagggcggctaccagctgattagcgtgttcgatcgctacattctgctggacgcccgcattcattttggcggtagccaattgcacgacgtctatatcggtccggatagccatctgctgctgtattacatgccggttgaggcgatccgtccggcggatcacattctggacctgtgtacgggtaccggcgttatcggcttgggtttgtcgcgctttagcgaacatgtggtcagcacggacattgcgccaccggcgctgcgcctggcgcacatgaaccgtgctctgaacaatgcggaaggccgtgtgagcattcgtgcggagaatttgcaggaaaccctggcgtccgatgaatgctttgacctgatcgcgtgcaacccgccgtatgtggcggcaccgccggagctgccgaccccgctgtatgcccagggcccagaccgtgatggcctgggttacctgcgtttgctgatggagcgtgccccggaaaaactgaacccgggtggtcaagcgatgttcgtggtggacctgattggcgacacgcaccgcccgtactatttcgacgatctggagcgtattgcgaaggagcaagagctgtttatcgaggcgttcatcgataaccgcctgaaggctgatggtcaactgccggcatataagttcctgtacgcacgtctgtttcatggtactcctccggaagaaatcgaacagcgtatgcgcaatttcatttttgacgagttgcacgcctactactattacatgaccacgctgcgtgtccgtcgccgtaaaccgagcggcttgcgtgttctggatcgctaccgcatcacgtcttacgatgagtttttccagcaatcttaa

SEQ ID NO: 93 - protein, 400 aa, Artificial Sequence; identical to SEQ ID NO: 11
  Synthetic Construct